(19) United States
(12) Patent Application Publication
(10) Pub. No.: US 2023/0118682 A1
(43) Pub. Date: Apr. 20, 2023

(54) MODIFIED J-AGGREGATES AND CONJUGATES THEREOF

(71) Applicant: GEORGE MASON UNIVERSITY, FAIRFAX, VA (US)

(72) Inventors: REMI VENEZIANO, MCLEAN, VA (US); PARAG VIJAY CHITNIS, BURKE, VA (US); SHRISHTI SINGH, FAIRFAX, VA (US)

(73) Assignee: GEORGE MASON UNIVERSITY, FAIRFAX, VA (US)

(21) Appl. No.: 17/937,490

(22) Filed: Oct. 3, 2022

Related U.S. Application Data
(60) Provisional application No. 63/251,832, filed on Oct. 4, 2021.

(51) Int. Cl. C09B 23/04

(52) U.S. Cl. CPC C09B 23/04; G03C 2001/089

(57) ABSTRACT

Provided is a modified J-aggregate (MJ). In some embodiments, the MJ is nanoscale, sub-micronscale, or microscale. Also provided is an MJ conjugate. Also provided are processes for making an MJ or an MJ conjugate. Photoacoustic imaging methods employing the MJ or MJ conjugate are also provided.

### CROSS-REFERENCE TO RELATED APPLICATIONS

The instant application is entitled to priority under 35 U.S.C. § 119(e) to U.S. Provisional Application No. 63/251,832, filed Oct. 4, 2021, which is hereby incorporated by reference in its entirety.

### FIELD

The general inventive concepts relate to the field of modified J-aggregates and conjugates thereof.

### SEQUENCE LISTING

The content of the electronic sequence listing (306564-00035_Sequence_Listing.xml; Size: 2,717 bytes; and Date of Creation: Sep. 29, 2022) is herein incorporated by reference in its entirety.

### BACKGROUND

Non-invasive biomedical imaging modalities such as magnetic resonance imaging (MRI) (1, 2), computed tomography (CT) (3-5), positron emission tomography (PET) (6), and photoacoustic imaging (PAI) (7-9) are useful tools for preclinical research and clinical applications. They have been used for diagnosis of diseases such as glioblastoma and ischemic heart disease (10, 11), early cancer detection (12, 13) and guided biopsy (14, 15). These techniques rely on endogenous or exogenous contrast agents or targeted imaging probes that can label specific tissues to create an identifiable image with high contrast (16-19). The relatively low cost of PAI, combined with its ability to provide functional images without the use of harmful radiation, make it attractive for preclinical research and point-of-care imaging.

PAI is a hybrid technique that combines the advantages of optical imaging (e.g., high contrast and molecular specificity) and ultrasound imaging (e.g., deep penetration in tissue). PAI relies on local absorption of pulsed laser light by tissues, and subsequent thermoelastic generation of ultrasound waves, which are detected using a transducer and combined to reconstruct the final image (20). PAI uses either endogenous contrast agents such as hemoglobin, melanin, or phospholipids in tissues (21) or exogenous probes such as fluorophores (22, 23), metallic nanoparticles (24, 25), or polymer-based nanomaterials (26, 27). PAI has been used for various biomedical applications, including imaging tumor lesions (28), detecting metastatic lymph nodes (29, 30), delineation of tumor margins (31, 32), identification of pathological tissues (33), and measurement of blood oxygenation level (34). Importantly, the use of exogenous near-infrared I (NIR-I) (620-950 nm) and II (1,000-1,700 nm) contrast agents further improves imaging depth by reducing the effects of photon scattering in turbid media such as tissue, which is inversely related to optical wavelength (35, 36).

However, clinically compatible long-wavelength contrast agents for PAI are currently lacking. An effective contrast agent for PAI must have minimal fluorescence emission to maximize the photothermal conversion efficiency and increase the photoacoustic (PA) signal. The contrast agent should also be amenable to functionalization with targeting modalities to increase its specificity. Several exogenous fluorophores, such as organic NIR cyanine dyes (37, 38), represent good candidates but often suffer from poor photothermal stability (39), and limited intrinsic targeting capabilities. Therefore, nanoscale delivery vehicles such as micelles or liposomes are required, which complicate formulation of such dye-based contrast agents (35, 36).

Aggregates of cyanine dyes with absorption in the NIR-I region represent a promising alternative to free monomeric dyes. Indeed, cyanine dye J-aggregates, which are stable head-tail arrangements of dye molecules held together by non-covalent interactions like van der Waals and electrostatic interactions can be used to assemble PAI contrast agents with increased photothermal stability (40) and strong PAI signal (41). J-aggregation of cyanine dyes results in a strong red (bathochromic) shift of the absorbance peak coupled with narrowing of the absorbance peak and a higher absorbance (42, 43). Among the cyanine dyes that can form J-aggregates, indocyanine green (ICG) is attractive for developing novel PAI probes because it is an FDA-approved NIR dye that can easily form J-aggregates (ICG-JA) in aqueous solutions at temperature above 50° C. (44) and at free dye concentrations greater than 1 mM. These ICG-JA exhibit a red-shifted absorption peak at a wavelength of 895 nm (45) and drastically reduced fluorescence upon excitation in the NIR-I spectrum (46). Because of their biocompatibility, enhanced photothermal stability relative to free ICG (45), and strong NIR absorption, ICG-JA have been explored for photoacoustic imaging applications, such as imaging diseased tissues like tumors (47, 48). However, these aggregates are generally highly polydisperse, poorly soluble, too large (a few microns) for many imaging applications, and lacking in intrinsic targeting capabilities, which limit their use as imaging probes. Currently, to create ICG-JA with in vivo targeting capabilities of nanometer size, most methods require encapsulation of the aggregates within targeted nanocarriers (40, 49). These methods require complicated chemistry and repeated purification steps (50, 51) and are constrained by the type of material used to assemble the nanocarriers (48). Thus, there still exists a critical need to assemble J-aggregates whose size can be easily tailored for imaging applications without using complicated synthesis methods or nanocarriers. Therefore, developing a facile method for direct functionalization of ICG J-aggregates with targeting molecules would avoid the complexities and limitations of nanocarrier encapsulation and enable high-throughput and cost-efficient synthesis of a targeted PAI contrast agent for use in the NIR regime.

There remains a need for improved photoacoustic probes and methods of producing photoacoustic probes, as well as methods of using photoacoustic probes.

### SUMMARY

Provided is a modified J-aggregate (MJ). In some embodiments, the MJ comprises a J-aggregate forming dye. In some embodiments, the MJ comprises a J-aggregate forming dye that has been functionalized. In some embodiments, the MJ comprises a J-aggregate forming dye and a J-aggregate forming dye that has been functionalized.

In some embodiments, the J-aggregate forming dye has been functionalized with an azide group.

In some embodiments, the J-aggregate forming dye is a cyanine dye. In further embodiments, the J-aggregate forming dye is selected from the group consisting of indocyanine green (ICG), ICG-NHS, ICG-NH₂, pseudoisocyanine, merocyanine, bis(2,4,6-trihydroxyphenyl)squaraine, tetrtakis(4-sulfonatophenyl)-porphyrin, antimony(III)-phthalocyanine, copper phthalocyanine, perylene bismide, hypericin, subphtalocyanine, and combinations thereof.

Provided is an MJ produced by the process of mixing a J-aggregate forming dye and a J-aggregate forming dye that has been functionalized. In some embodiments, the mixing a J-aggregate forming dye and a J-aggregate forming dye that has been functionalized occurs in the presence of a salt. In further embodiments, the salt is selected from the group consisting of KCl, NaCl, MgCl₂, CaCl₂, NiCl₂, MnCl₂, EuCl₃, TbCl₃, and a salt from a rare earth element. In some embodiments, the salt from a rare earth element is a lanthanide salt, such as PrCl₃, or EuCl₃.

In some embodiments, the MJ is nanoscale, sub-micronscale, or microscale. In further embodiments, the MJ is nanoscale.

Provided is an MJ conjugate of Formula I

MJ—E—D

Formula I

wherein

MJ is a modified J-aggregate;

E is a linker or a bond;

D is a functional moiety.

In some embodiments, the MJ comprises a J-aggregate forming dye.

In some embodiments, the MJ comprises a J-aggregate forming dye that has been functionalized.

In some embodiments, the MJ comprises a J-aggregate forming dye and a J-aggregate forming dye that has been functionalized.

In some embodiments, the J-aggregate forming dye has been functionalized with an azide group. In further embodiments, D has been attached to MJ by employing copper-free click chemistry with said azide group.

In some embodiments, the J-aggregate forming dye is a cyanine dye. In further embodiments, the J-aggregate forming dye is selected from the group consisting of indocyanine green (ICG), ICG-NHS, ICG-NH₂, pseudoisocyanine, merocyanine, bis(2,4,6-trihydroxyphenyl)squaraine, tetrtakis(4-sulfonatophenyl)-porphyrin, antimony(III)-phthalocyanine, copper phthalocyanine, perylene bismide, hypericin, subphtalocyanine, and combinations thereof.

In some embodiments, the MJ was produced by the process of mixing a J-aggregate forming dye and a J-aggregate forming dye that has been functionalized. In further embodiments, the mixing a J-aggregate forming dye and a J-aggregate forming dye that has been functionalized occurs in the presence of a salt. In further embodiments, the salt is selected from the group consisting of KCl, NaCl, MgCl₂, CaCl₂, NiCl₂, MnCl₂, EuCl₃, TbCl₃, NaOH, K₂PO₄, Mn(NO₃)₂, Ni(NO₃)₂ and a salt from a rare earth element. In some embodiments, the salt from a rare earth element is a lanthanide salt, such as PrCl₃, or EuCl₃.

In some embodiments, E is a linker comprising a copper-free click chemistry group. In further embodiments, E is a linker selected from the group consisting of DBCO-PEG-NHS, DBCO-PEG-Biotin, DBCO-PEG-Thiol, DBCO-PEG-NH₂, DBCO-PEG-Mal, Thiol-PEG-NHS, Thiol-PEG-Biotin, Thiol-PEG-NH₂, Mal-PEG-NHS, Mal-PEG-NH₂, Mal-PEG-Acrylate, Thiol-PEG-Acrylate, DBCO-PEG-Folate, and combinations thereof.

In some embodiments, D comprises streptavidin, biotin, RGD, folate, or a derivative thereof. In some embodiments, D comprises a drug, a protein, a targeting protein, or an antibody. In further embodiments, the drug, protein, targeting protein or antibody is connected to E via a biotin-streptavidin interaction. In some embodiment, E comprises biotin and D comprises streptavidin. In some embodiments, E comprises streptavidin and D comprises biotin.

In some embodiments, the drug is a chemotherapeutic (e.g., Doxorubicin, Daunorobicin). In some embodiments, the drug is a steroid, a chemokine, a cytokine, a hormone, a therapeutic protein, an antimicrobial peptide, or a nucleic acid. In some embodiments, the nucleic acid is mRNA or siRNA. In some embodiments, the drug is bradykinin (BK).

In some embodiments, the protein is avidin, streptavidin, neutravidin, an enzyme, a signal peptide, a marker, a growth factor, a neurotransmitter, an antibody, an interferon, thrombin, an anticoagulant protein, a targeting peptide, or a cell penentrating peptide. In further embodiments, the antibody is a broad neutralizing antibody. In further embodiments, the anticoagulant protein is Protein C, Protein S or antithrombin.

In some embodiments, the targeting protein is a receptor, for example a chemokine receptor, a cytokine receptor, a hormone receptor, a growth factor receptor, a neurotransmitter receptor, or a cell adhesion receptor.

In some embodiments, the antibody is an antibody to a target tissue or an antibody to a target deep tissue. In some embodiments, the antibody is a monoclonal antibody, a polyclonal antibody, or a single domain antibody. In some embodiments, the antibody is anti-CTL4-4, anti-VCAM 1, anti-asialoglycoprotein receptor, or anti-VEGFR. In some embodiments, the monoclonal antibody is anti-CTL4-4, for example for targeting T lymphocytes. In some embodiments, the polyclonal antibody is anti-VCAM 1, for example for targeting kidney tissues. In some embodiments, the single domain antibody is anti-asialoglycoprotein receptor, for example for targeting liver tissues. In some embodiments, the single domain antibody is anti-VEGFR, for example a nanobody anti-VEGFR.

Provided is a method of photoacoustic imaging at a target site comprising providing the MJ of any one of the previous embodiments at the target site; and monitoring absorbance at the target site.

Provided is a method of photoacoustic imaging at a target site comprising providing the MJ conjugate of any one of previous embodiments at the target site; and monitoring absorbance at the target site.

### DESCRIPTION OF THE FIGURES

FIGS. 1A-1B show synthesis of ICG J-aggregates (ICG-JA) and azide-modified ICG J-aggregates (JAAZ) microparticles. FIG. 1A: Schematic representation of the procedure developed to synthesize ICG-JA and JAAZ microparticles using ICG and ICG-azide. FIG. 1B: (top) Absorbance spectra of ICG-JA and JAAZ microparticles with UV-Vis-NIR spectroscopy. A sharp absorption peak at 895 nm and reduced monomer and dimer peaks at 780 nm and 715 nm, respectively, indicate successful formation of JA and JAAZ. Representative absorbance reading of free ICG dye, ICG-JA and JAAZ taken at 50 μM of free dye. A change in color of the solution is evident after formation of ICG-JA and JAAZ microparticles due to the shift toward longer wavelength.

FIGS. 2A-2F show characterization of JAAZ microparticles. FIG. 2A: Zeta potential measurements of ICG-JA and JAAZ filtered microparticles shows incorporation of relatively more positive ICG-azide compared to ICG with a decrease of the surface charge from −60.11 mV for JA to −50.32 mV for JAAZ (***=p<0.001, n=5 distinct replicates; all P values provided in Table 3). FIG. 2B: Dynamic light scattering measurements of ICG-JA and JAAZ particles, prepared in 20 mM KCl, show a slight but statistically significant increase (18%) of the hydrodynamic diameter of JAAZ in comparison with the ICG-JA particles. (*=p<0.05, n=5 distinct replicates; all P values provided in Table 3). FIG. 2C: SEM images of JAAZ microparticles prepared in 20 mM KCl at 1 mM dye concentration. (Scale bar: 2 μm, the complete SEM image is provided in FIG. 13). FIG. 2D: UV-Vis-NIR absorbance of JAAZ microparticles incubated in solutions of pH 5, 7, and 9 at 37° C. for 126 hours. FIG. 2E: Absorbance of JAAZ microparticles incubated in DMEM and DMEM+10% FBS for 48 hours at 37° C. (n=3). The peak visible at 560 nm is due to DMEM media. FIG. 2F: Percentage stability of JAAZ particles in DMEM, DMEM+10% FBS and 100% FBS. Stability over time was quantified as difference in the intensity of the 895 nm absorption peak when compared to control at 0 minutes.

FIGS. 3A-3D show functionalization of the JAAZ microparticles. FIG. 3A: Schematic representation of the procedure developed to attach streptavidin to the JAAZ microparticles. i. JAAZ particles are assembled and purified. ii. Biotin-PEG-DBCO is attached to the azide groups via click chemistry to form Bio-JAAZ. iii. After purification of the Bio-JAAZ to remove the excess of biotin linker, streptavidin at 10X (molar ratio) is attached to the biotin group and subsequently purified to form the Strep-JAAZ. iv. The introduced streptavidin can then be used to attach any biotinylated biomolecules such as targeting peptides like RGD, fluorophores, and or antibodies. FIG. 3B: Absorbance of different functionalized samples (JAAZ, Bio-JAAZ, Strep-JAAZ and RGD-JAAZ) after filtration with intact 895 nm absorbance peak. Absorbance value measured at 25 μM of free dye (n=1). FIG. 3C: Zeta potential measurements of the JAAZ, Bio-JAAZ, Strep-JAAZ, and RGD-JAAZ clearly show a change of surface charge when modified with biomolecules (n=3 distinct replicates, **=p<0.01, ****=p<0.0001; all P values provided in Table 4). FIG. 3D: SEM images and EDX analysis of streptavidin coated JAAZ particles (Scale bar: 1 μm).

FIGS. 4A-4F show characterization of N-JAAZ. FIG. 4A: UV-Vis-NIR spectrum of JAAZ nanoparticles formed in 0.1 mM KCl using 1:10 ICG-azide:ICG solutions at 250 and 500 μM of total dye. Representative absorbance is of N-JAAZ particles formed at 14 hours of incubation. The size of the formed N-JAAZ at 8, 14 and 20 hours at the two dye concentrations is seen in inset. FIG. 4B: Size of nanoscale, sub-micron, and microscale JAAZ particles formed by incubation in different molarities of KCl. The concentration of dye used for 0.1 mM samples was 250 μM and 500 μM for 1 and 20 mM KCl samples (n=4 distinct replicates, ****=p<0.0001, ***=p<0.001; all P values provided in Table 5). FIG. 4C: Agarose gel electrophoresis of the JAAZ and N-JAAZ. The nanoscale aggregates migrate through the gel and form a sharp green band whereas the larger JAAZ particles are concentrated in the well (n=1). FIG. 4D: SEM images of N-JAAZ particles prepared in 0.1 mM KCl with 250 μM of dye. The scale bar in the images is 250 nm and the entire image is provided in FIG. 24. FIG. 4E: The size of N-JAAZ and its modified forms. The size of N-Bio-JAAZ, N-Strep-JAAZ and N-RGD-JAAZ are 206.6 nm, 256.5 and 313.0 nm, respectively. The size difference is non-significant (n=4 distinct replicates; ns, not significant; all P values are provided in Table 5). FIG. 4F: Zeta potential values of N-JAAZ, N-Bio-JAAZ and N-Strep-JAAZ are −52 mV, −53.1 mV and −20.6 mV respectively (n=4, ***=p<0.001, ns, not significant; all P values are provided in Table 5).

FIGS. 5A-5F show photoacoustic properties of various JA particles. FIG. 5A: Picture of the photoacoustic set-up with pulsed laser, fixed transducer and reservoir containing deionized water in which the 96-well plate is submerged. FIG. 5B: PA signal of the different JA particles, namely JAAZ, N-JAAZ and RGD-JAAZ at different concentrations (25, 12.5 and 5 μM; blue, orange, and green respectively) in 0.5% agarose normalized with respect to 1:100 (v/v) dilution of India Ink (n=100, ****=p<0.0001, ns, not significant; all P values are provided in Table 6). Individual data points are not shown for simplicity. FIG. 5C: Normalized absorbance (dashed line) and normalized photoacoustic spectra (continuous line) of JAAZ, N-JAAZ and RGD-JAAZ particles in 0.5% agarose. FIG. 5D: Photoacoustic signal of ICG dye and different J-aggregates, namely JAAZ, N-JAAZ and RGD-JAAZ measured in whole sheep's blood. The concentration of all samples was 20 μM and all values were normalized with respect to whole blood (n=100, ****=p<0.0001, ns, not significant; all P values are provided in Table 6). Individual data points are not shown for simplicity. FIG. 5E: 2D photoacoustic image of HeLa cells stained with 10 μM JAAZ and RGD-JAAZ particles (post-processed with a 2D gaussian filter) showing photoacoustic signal normalized to maximum value. An area of the glass slide was scraped with a razor blade to remove cells before staining. Images were acquired by raster-scanning 35 MHz transducer at 100 μm steps. Representative scale of PA signal amplitude is also given in the figure. FIG. 5F: Representation of azide functionalized J-aggregates with tunable size assembled with a mix of ICG and ICG-azide dyes. Various targeting moieties can be tethered to these J-aggregates for efficient cell targeting and use as a photoacoustic imaging probe.

FIGS. 6A-6B show formation of ICG-JA in water. FIG. 6A: Absorbance measurements of ICG-JA formed in water at temperatures of 50° C., 60° C., and 70° C. for 20 hours (n=3). FIG. 6B: UV-Visible-NIR spectrum of ICG-JA formation at 60° C. for different time points in water (n=3). Absorbance measurements taken at 25 μM equivalent of free dye. The monomer and dimer peaks are indicated in each figure at 780 and 715 nm respectively. The J-aggregate peak is indicated at 895 nm.

FIGS. 7A-7B show that ICG-JA form in pure water, but JAAZ do not. FIG. 7A: UV-Visible-NIR spectrum of 100% ICG-azide, 100% ICG dye and different molar ratio solutions of ICG-azide:ICG made in water after 20 hours of incubation (n=2). FIG. 7B: Absorbance readings after 40 hours of incubation (n=2). Absorbance measurements taken at 25 μM free dye concentration.

FIG. 8 shows J-aggregates formed from ICG-azide:ICG mixture in pure water. FT-IR spectrum of ICG-JA particles with 1:10 ICG-azide:ICG molar ratio formed in water (n=2) at 60° C. The lack of a peak between 2141-2100 cm⁻¹ indicates that no azide was incorporated into the J-aggregates.

FIGS. 9A-9D show formation of JAAZ in KCl. (FIGS. 9A-9D) UV-Visible-NIR spectra of 1:10 ICG-azide:ICG molar solution at dye concentration of 1 mM in different KCl concentrations of 1 mM (FIG. 9A), 5 mM (FIG. 9B), 10 mM (FIG. 9C) and 20 mM (FIG. 9D) incubated for 20 hours at 60° C. (n=3).

FIGS. 10A-10D show UV-Vis-NIR and FT-IR spectrum of different molar ratio JAAZ FIG. 10A: UV-Vis-NIR spectrum of JAAZ particles formed with different molar solutions of ICG-azide:ICG (1:10, 1:5, 1:3) to a final dye concentration of 1 mM. Absorbance readings taken at a dye concentration of 50 μM (n=3). FIGS. 10B-10D: FT-IR spectrum of JAAZ particles with (FIG. 10B) 1:10 (orange), (FIG. 10C) 1:5 (black), and (FIG. 10D) 1:3 (green) molar ratio of ICG-azide:ICG. The FT-IR spectrum of the control is ICG-JA particles is depicted in blue (n=1).

FIGS. 11A-11B show filtration and fluorescence of ICG-JA and JAAZ. FIG. 11A: UV-Vis-NIR spectra of the formed ICG-JA and JAAZ before and after filtration showing a clear peak 895 nm, typical of ICG J-aggregates. Absorbance measurements taken at 25 μM free dye concentration (n=2). FIG. 11B: Fluorescence emission curves of free ICG dye at concentrations 5, 2.5 and 1.25 μM and those of ICG-JA, JAAZ, N-JAAZ and RGD-JAAZ particles. Emission is absent in the various J-aggregate particles, a typical characteristic of J-aggregates.

FIG. 12A shows Zeta potential distribution of ICG-JA and JAAZ. Charge distribution profile of ICG-JA and JAAZ showing a sharp and narrow profile centered at −61 mV for JA and −52 mV for JAAZ (n=3). FIG. 12B shows rate of formation of JAAZ particles at different temperatures. Absorbance intensity at 895 nm of the JAAZ particles formed at 50° C. (circles), 60° C. (squares) and 70° C. (triangles). The final dye concentration in all samples is 1 mM.

FIGS. 13A-13B show SEM image and EDX analysis of JAAZ particles. FIG. 13A: SEM image of JAAZ particles prepared at 1 mM dye concentration and 20 mM KCl. Size measurements of these JAAZ particles using DLS showed an average of 900 nm. FIG. 13B: EDX analysis showing weight percentage of carbon (C), nitrogen (N), oxygen (O), sulfur (S), potassium (K) and chloride (Cl) of five different JAAZ particles as marked in FIG. 13A.

FIGS. 14A-14C show drying and resuspension of JAAZ particles. FIG. 14A: The absorbance curves of JAAZ and N-JAAZ particles before drying and after resuspension (n=2). Absorbance measurements taken at 25 μM of free dye. FIG. 14B: Absorbance value at 895 nm of JAAZ particles incubated in pH 5, 7 and 9 for 126 hours at 37° C. FIG. 14C: Absorbance curves of JAAZ particles before and after they are incubated in two different detergents (Tween-20 and Triton X-100) at 37° C. (n=2), which causes them to dissociate into free dye as indicated by the disappearance of the 895-nm peak and reappearance of the monomer and dimer peaks. Absorbance measurements taken at 25 μM of free dye.

FIGS. 15A-15B show a Standard Curve of ICG dye and percentage recovery of J-aggregates. FIG. 15A: Standard Curve of ICG dye monomer or free ICG dye used to determine the free dye concentration in disassociated ICG-JA and JAAZ particles. FIG. 15B: Percentage recovery of different JAAZ samples (Bio-JAAZ, black; Strep-JAAZ, dark gray; RGD-JAAZ, light gray) when compared to JAAZ. (n=4 distinct replicates, p=ns, *=p<0.05, not significant; all P values are provided in Table 7).

FIGS. 16A-16B show size and PDI of Streptavidin functionalized JAAZ. FIG. 16A: The size of the different biomolecule modified forms of JAAZ (n=5 distinct replicates, p=ns, not significant; all P values are provided in Table 8). FIG. 16B: The PDI of JAAZ, Bio-JAAZ, Strep-JAAZ and RGD-JAAZ was 0.36, 0.33, 0.27 and 0.26 respectively. (n=5 distinct replicates, p=ns, not significant; all P values are provided in Table 8).

FIGS. 17A-17B show UV-Vis-NIR spectrum of different molar ratio Strep-JAAZ particles. FIG. 17A: UV-Vis-NIR spectrum of different ICG-azide:ICG molar ratio JAAZ particles modified with streptavidin (n=3). Absorbance measurement taken at 25 μM of free dye. FIG. 17B: The zeta potential of 1:20, 1:10, 1:5 and 1:3 Strep-JAAZ samples are −16.4, −13.3, −15.5 and −14.1 respectively (shown in inset, n=3 distinct replicates, p=ns, not significant; all P values are provided in Table 9).

FIGS. 18A-18B show sedimentation of JAAZ and Strep-JAAZ for 24 hours. FIG. 18A: Pictures of JAAZ sample at 0, 1, 2, 4, 6, 8 and 24 hours of sedimentation on a benchtop at room temperature. FIG. 18B: Pictures of streptavidin-modified JAAZ (Strep-JAAZ) sample at 0, 1, 2, 4, 6, 8 and 24 hours of sedimentation on a benchtop at room temperature. Both samples are filtered and at a free dye concentration of 200 μM. The volumes of both samples are 50 μL. (n=1).

FIGS. 19A-19D show formation of JAAZ in 0.1 mM KCl at different ICG dye concentrations. FIGS. 19A-19D: UV-Vis-NIR spectrums of 1:10 ICG azide:ICG molar solution in 0.1 mM KCl concentration at 1000 μM (FIG. 19A), 750 μM (FIG. 19B), 500 μM (FIG. 19C) and 250 μM (FIG. 19D) total dye concentrations depicted for 20 hours.

FIGS. 20A-20D show formation of JAAZ in 1 mM KCl at different ICG dye concentrations. (FIGS. 20A-20D) UV-Vis-NIR spectrums of 1:10 ICG azide:ICG molar solution in 1 mM KCl concentration at 1000 μM (FIG. 20A), 750 μM (FIG. 20B), 500 μM (FIG. 20C) and 250 μM (FIG. 20D) total dye concentrations depicted for 20 hours.

FIGS. 21A-21D show formation of JAAZ in 20 mM KCl at different ICG dye concentrations. FIGS. 21A-21D: UV-Vis-NIR spectrums of 1:10 ICG-azide:ICG molar solution in 20 mM KCl concentration at 1000 μM (FIG. 21A), 750 μM (FIG. 21B), 500 μM (FIG. 21C) and 250 μM (FIG. 21D) total dye concentrations depicted for 20 hours.

FIGS. 22A-22D show size of JAAZ in different KCl concentrations. FIGS. 22A-22D: Average size formed JAAZ of 1:10 molar solutions at 1000 μM (FIG. 22A), 750 μM (FIG. 22B), 500 μM (FIG. 22C) and 250 μM (FIG. 22D) total dye concentrations depicted for 20 hours. (n=3 distinct replicates, *=p<0.05, **=p<0.01, ***=p<0.001, ****=p<0.0001; all P values are provided in Table 10).

FIG. 23 shows a summary of effects of KCl concentration, dye concentration, and time on the average size of JAAZ particles. The summary represents the range of sizes of JAAZ particles formed at different KCl concentrations with increasing time and increasing total dye concentration. A color bar representing the different sizes range (in nm) is also given.

FIGS. 24A-24C show SEM image and EDX analysis of N-JAAZ particles. FIG. 24A: SEM image of N-JAAZ particles prepared at 250 μM dye concentration and 0.1 mM KCl. Size measurements of N-JAAZ particles using DLS showed an average of 183 nm. FIG. 24B: EDX analysis showing weight percentage of carbon (C), nitrogen (N), oxygen (O), sulfur (S), potassium (K) and chloride (Cl) of five different N-JAAZ particles as marked in FIG. 24A. FIG. 24C: Chemical structures of ICG and ICG-azide dye are shown for reference.

FIG. 25 shows stability of N-JAAZ particles in media. UV-Vis-NIR absorbance spectra of nanosized JAAZ particles after incubation in DMEM and DMEM supplemented with 10% FBS. The samples were incubated for 48 hours at 37° C. The absorbance readings are recorded at 25 μM of free dye concentration.

FIGS. 26A-26C show UV-Vis-NIR spectra of different JA samples. FIG. 26A: Absorbance spectrum of JAAZ microparticles at 25, 12 and 5 μM of free dye concentration. FIG. 26B: Absorbance spectrum of nanoscale JAAZ (N-JAAZ) at 25, 12 and 5 μM of free dye concentration. FIG. 26C: Absorbance spectrum of peptide modified JAAZ (RGD-JAAZ) at 12 and 5 μM of free dye concentration. Absorbance reading of all samples is taken at 120 μl(n=2 distinct replicates).

FIGS. 27A-27B show photobleaching and scan of a pattern using RGD-JAAZ. FIG. 27A: PA signal intensity of 10 μM RGD-JAAZ microparticles under maximum laser power. FIG. 27B: 2D photoacoustic image of ‘M’ pattern made with 10 μM RGD-JAAZ microparticles.

FIGS. 28A-28C show pictures of HeLa cells stained with JAAZ and RGD-JAAZ and the corresponding PA intensity of the stained cells. FIG. 28A: Image of a glass slide with a red silicone insert creating a well containing HeLa cells stained with 10 μm JAAZ particles. FIG. 28B: Image of a glass slide with a red silicone insert creating a well containing HeLa cells stained with 10 μm RGD-JAAZ particles. FIG. 28C: Maximum PA signal from a 100 μm area of stained cells from both JAAZ and RGD-JAAZ stained glass slides at varying laser powers (100%, 95%, 90%, 80%, and 60%).

FIGS. 29A-29B show formation of different sized azide-modified J-aggregates. FIG. 29A: Schematic of formation of different sized azide-modified J-aggregates. FIG. 29B: Graphical representation showing decreasing trend in the size of J-aggregates composed of ICG and ICG-azide (ICG-N₃) dyes. A combination of increasing KCl concentration (in mM) and increasing ratio of ICG-N₃:ICG yields monodisperse, nanosized J-aggregates as shown in the size intensity distribution graph. The total concentration of dye in all samples is 350 μM.

FIGS. 30A-30D show characterization and stability of N-JAAZ particles. FIG. 30A: Absorbance spectrum of N-JAAZ particles with varying ICG-azide:ICG molar ratio formed in 5 mM KCl. FIG. 30B: Size of the formed N-JAAZ particles and gel electrophoresis of the samples. FIG. 30C: SEM images and EDS spectra (below) of the N-JAAZ particles. Scale bar is 250 nm. FIG. 30D: Stability of the N-JAAZ particles in different buffers such as 1X PBS, DMEM+10% FBS and serum. Absorbance and stability of the N-JAAZ particles for 185 days (below). (n=3 for a, b and d (above), p=****=0.0001).

FIGS. 31A-31C show functionalization of N-JAAZ particles. FIG. 31A: Schematic of copper-free click chemistry wherein compounds such as DBCO-PEG-Folate link onto the available azide groups (in gray) on the N-JAAZ particles. FIG. 31B: Zeta potential magnitude of different molar ratio bare and DBCO-PEG-Folate functionalized N-JAAZ particles. FIG. 31C: Size of the different molar ratio JAAZ particles before and after functionalization with DBCO-PEG-Folate. (Inset) UV-Vis-NIR curves of all molar ratio N-JAAZ particles after folate attachment. (n=3 for b and c (above), p=ns=non-significant, p=**=0.001, p=****=0.0001).

FIGS. 32A-32C show photoacoustic properties of N-JAAZ particles. FIG. 32A: Photoacoustic signal magnitude of bare N-JAAZ and folate functionalized particles at different equivalent concentration of free dye. The data is normalized with respect to 1:100 ratio of India Ink used as a control. FIG. 32B: PA spectrum (solid lines) and absorbance spectrum (dashed lines) of bare and functionalized N-JAAZ particles. FIG. 32C: PA signal magnitude of bare and functionalized N-JAAZ particles mixed in whole blood. The signal is normalized with respect to whole blood.

FIGS. 33A-33D show UV-Vis-NIR spectrum of different molar ratios of ICG-azide:ICG namely 1:10, 1:5, 1:3, 2:5 and 1:1 in (FIG. 33A) 1 mM KCl, (FIG. 33B) 2 mM KCl, (FIG. 33C) 3 mM KCl and (FIG. 33D) 4 mM KCl. All absorbance readings were taken at a volume of 60 μl (n=3 distinct replicates).

FIGS. 34A-34F show Hydrodynamic diameters measured of J-aggregates synthesized with (FIG. 34A) 1:10, (FIG. 34B) 1:5, (FIG. 34C) 1:3, (FIG. 34D) 2:5 and (FIG. 34E) 1:1 ICG-azide:ICG molar ratios with different KCl molarities namely 1, 2, 3, 4 and 5 mM KCl. The total concentration of dye in all solutions was 350 μM. FIG. 34F: A schematic summary of the different sized J-aggregates synthesized with varying salt and ICG-N₃:ICG molar ratios.

FIGS. 35A-35F show UV-Vis-NIR spectra of (a) 1:3, (b) 2:5, and (c) 1:1 ICG-azide:ICG molar ratio samples formed after 6, 8, 12 and 18 hours of incubation. (d-f) Average size of 1:3, 2:5 and 1:1 molar ratio samples after 18 hours of incubation. (n=3 distinct replicates).

FIGS. 36A-36C show UV-Vis-NIR spectrum of nanosized J-aggregates incubated in (FIG. 36A) DMEM+10% FBS, (FIG. 36B) 100% FBS and (FIG. 36C) 1XPBS for 48 hours at 37° C. All absorbance readings were taken at an equivalent dye concentration of 15 μM and 60 μl volume. (n=3 distinct replicates).

FIGS. 37A-37C show properties of functionalized N-JAAZ particles. FIG. 37A: Zeta potential of 1:3 ICG-azide:ICG molar ratio N-JAAZ particles modified with biotin followed by streptavidin. FIG. 37B: Hydrodynamic diameter of N-JAAZ particles after functionalization with biotin and streptavidin. FIG. 37C: Zeta potential measurements of different molar ratio N-JAAZ particles before (circle) and after functionalization (square) with DBCO-NTA.

FIGS. 38A-38B show formation of J-aggregates with ICG and modified ICG dyes. FIG. 38A: UV-Vis-NIR spectrums demonstrating that the 895 nm J-aggregate peak is formed when ICG dye is incubated with ICG-NHS (black) or ICG-NH₂ (grey). FIG. 38B: The hydrodynamic diameter measured using DLS of the J-aggregates formed by mixing ICG with ICG-NHS or ICG-NH₂. The average diameter of ICG+ICG−NHS J-aggregates was 734±54 nm and that of ICG+ICG-NH₂ was 304±39 nm.

FIGS. 39A-39E show in vivo imaging using RGD-JAAZ particles. FIG. 39A: Schematic and photograph of the 3D PAI acquisition on the TriTom™ imaging system. FIG. 39B: Coronal and axial slabs of the baseline PA signal acquired at 895 nm. FIG. 39C: Representative coronal and axial slabs of the ICG (left) and RGD-JAAZ particle (right) injections acquired at the peak excitation wavelength (i.e., 800 and 895 nm, respectively) over time. The displayed dynamic range for all images was set based on the baseline scan acquired at the same wavelength. (Legend): a. superficial thoracic vessels, b. liver c. spleen d. intestines e. thoracic vertebrae f. CuSO₄ fiducial tube. FIG. 39D: Axial slabs encompassing the iliac arteries (white ROI) used to calculate the achievable imaging depth for ICG and RGD-JAAZ particles 90-minutes post-injection. FIG. 39E: For each panel, the left image shows coronal slabs of oxyhemoglobin (red), deoxyhemoglobin (blue). For each panel, the right image shows RGD-JAAZ particles (green). Spectral unmixing is overlaid on the 800 nm excitation scan of anatomy (white) at 0- (left panel) and 90-minutes (right panel) post-injection. Scale bars for all images are 10 mm. (n=2 female nu/nu nude mice).

FIG. 40 shows phantom imaging of RGD-JAAZ particles. Measured PA spectra of 0.4 mM RGD-JAAZ particles and monomeric ICG dye acquired with the TriTom™ imaging system.

FIGS. 41A-41B show in vivo imaging using RGD-JAAZ particles. FIG. 41A: Coronal and axial slabs of the ICG (left) and RGD-JAAZ particle (right) injections acquired at the peak excitation wavelength (i.e., 800 and 895 nm, respectively) over time. The displayed dynamic range for all images was set based on the baseline scan acquired at the same wavelength. a. superficial thoracic vessels, b. liver c. spleen d. intestines e. thoracic vertebrae f. CuSO₄ fiducial tube. FIG. 41B: Representative coronal and axial slab of the RGD-JAAZ particle injection acquired at the peak excitation wavelength of 895 nm at 120 minutes. a. superficial thoracic vessels, b. liver c. spleen d. intestines e. thoracic vertebrae f. CuSO₄ fiducial tube. Scale bar for FIG. 41B is 10 mm.

FIG. 42 shows percent stability of N-JAAZ particles in media and serum. Stability of N-JAAZ particles incubated in DMEM (blue), DMEM+10% FBS (orange) and 100% FBS (black) at 37° C. The magnitude of the 895 nm absorbance peak was used to determine the stability of the particles. N-JAAZ particles showed ˜20% decrease in the 895 nm peak at 24 hours of incubation in 100% FBS. (data represented as mean±SD, n=3 distinct replicates).

FIGS. 43A-43B show Hemotoxicity and cytotoxicity of JAAZ particles. FIG. 43A: Optical microscopy images of red blood cells (RBCs) incubated with JAAZ (930±27 nm), N-JAAZ (256±21 nm) and RGD-JAAZ (997±48 nm) particles. There were no morphological changes observed of the RBCs between the control, untreated red blood cells and the treated cells. FIG. 43B: MTT assay results of HeLa cells incubated with 20 μm equivalent dye concentration JAAZ, N-JAAZ and RGD-JAAZ particles (data presented as mean±SD, n=3, p-values are calculated using one-way ANOVA, ns=p=0.9531).

FIG. 44 shows PA signal amplitude of different sized JAAZ particles. PA signal of JAAZ particles of different sizes along with N-JAAZ particles embedded in 0.5% (w/v) agarose. The sizes of the compared particles are 256±20 nm, 363±90 nm, 570±52 nm and 930±27 nm. The PA signal is normalized with respect to ICG-JA particles. All samples are at 2.0 absorbance OD. (data presented as mean±SD, n=100, p-values are calculated using one-way ANOVA, ***=p<0.001).

FIG. 45 shows percentage stability of JAAZ particles in whole blood. JAAZ, N-JAAZ and RGD-JAAZ particles at 10 μM equivalent free dye were incubated in whole sheep's blood at 37° C. for 24 hours. The magnitude of the 895 nm absorbance peak was used to determine the stability of the particles. RGD-JAAZ particles remained stable for 24 hours whereas N-JAAZ particles observed ˜20% degradation at 24 hours of incubation. JAAZ particles showed ˜30% decrease in the 895 nm peak 4 hours after incubation and were 60% stable after 24 hours. (data represented as mean±SD, n=3 distinct replicates).

FIG. 46 shows the principle of tumor margin staining after resection.

FIG. 47 shows NIR-II emission of contrast agent JAAZ (“Jagg”) in comparisong with free ICG. Excitation wavelength: 808 nm. Spectral range detected 1000-1700 nm (long-pass emission filter with a cutoff of 1000 nm). Left panel shows a graph of intensity. Right panel shows left: JAAZ (“Jagg”); right: Free ICG.

### DETAILED DESCRIPTION

While the general inventive concepts are susceptible of embodiment in many forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

It is also to be understood that the terminology used herein is for the purpose of describing particular embodiments only, and is not intended to be limiting.

The articles “a” and “an” are used herein to refer to one or more than one (i.e., to at least one) of the grammatical object of the article. By way of example, “a cell” means one cell or more than one cell.

“About” as used herein when referring to a measurable value such as an amount, a temporal duration, and the like, is meant to encompass variations of ±5%, preferably ±1%, and still more preferably ±0.1% from the specified value, as such variations are appropriate to perform the disclosed methods.

As used herein, the term “nanoscale” means particles having a size range between approximately 1 and 100 nanometers (nm).

As used herein, the term “nanometer” means 1/1,000,000,000 meter (m).

As used herein, the term “sub-micronscale” means particles having a size less than a micron (μm) and larger than 100 nm.

As used herein, the term “micron” means 1/1,000,000 meter (m).

As used herein, the term “microscale” means having a size of approximately 1 to 5 microns (μm).

As used herein, the term “RGD” means a common peptide responsible for cell adhesion comprising arginine, glycine and aspartic acid, or a derivative of the peptide, for example a derivative comprising biotin. RGD examples include, but are not limited to, Cyclo-Arg-Gly-Asp-D-Phe-Lys(Biotin) (c[RGDfK(Biotin)]) (SEQ ID NO: 1), which is commercially available from Vivitide (Louisville, Ky., USA), Sigma Aldrich (St. Louis, Mo., USA), Anaspec (Fremont, Calif., USA), or other vendors.

As used herein, the term “antibody” and “antibodies” are meant in a broad sense and include immunoglobulin molecules including polyclonal antibodies, monoclonal antibodies including murine, human, human-adapted, humanized and chimeric monoclonal antibodies, antibody fragments (e.g. F(ab)₂, Fab, Fv, or a domain antibody (dAb) fragment), bispecific or multispecific antibodies, dimeric, tetrameric or multimeric antibodies, diabodies, nobodies, and single chain antibodies (e.g. scFv).

In some embodiments of any of the compositions or methods described herein, a range is intended to comprise every integer or fraction or value within the range.

Embodiments described herein as “comprising” one or more features may also be considered as disclosure of the corresponding embodiments “consisting of and/or “consisting essentially of such features.

Thus, there still exists a critical need to assemble J-aggregates whose size can be easily tailored for imaging applications without using complicated synthesis methods or nanocarriers. Therefore, developing a facile method for direct functionalization of ICG J-aggregates with targeting molecules would avoid the complexities and limitations of nanocarrier encapsulation and enable high-throughput and cost-efficient synthesis of a targeted PAI contrast agent for use in the NIR regime.

Modified J-aggregates (MJs) described herein are designed to provide improved photoacoustic probes of various sizes, including nanoscale. Functional moieties, including but not limited to a drug, a protein, an antibody or a payload may be attached to the MJs either directly or via a linker to form MJ conjugates. Also provided herewith are methods of producing photoacoustic probes, as well as methods of using photoacoustic probes.

Modified J-Aggregates

Provided is a modified J-aggregate (MJ). In some embodiments, the MJ comprises a J-aggregate forming dye. In some embodiments, the MJ comprises a J-aggregate forming dye that has been functionalized. In some embodiments, the MJ comprises a J-aggregate forming dye and a J-aggregate forming dye that has been functionalized.

In some embodiments, the J-aggregate forming dye has been functionalized with an azide group.

In some embodiments, the J-aggregate forming dye is a cyanine dye. In further embodiments, the J-aggregate forming dye is selected from the group consisting of indocyanine green (ICG), ICG-NHS, ICG-NH₂, pseudoisocyanine, merocyanine, bis(2,4,6-trihydroxyphenyl)squaraine, tetrtakis(4-sulfonatophenyl)-porphyrin, antimony(III)-phthalocyanine, copper phthalocyanine, perylene bismide, hypericin, subphtalocyanine, and combinations thereof.

Provided is an MJ produced by the process of mixing a J-aggregate forming dye and a J-aggregate forming dye that has been functionalized. The J-aggregate forming dye and the J-aggregate forming dye that has been functionalized may be mixed at various molar ratios.

In some embodiments, the mixing a J-aggregate forming dye and a J-aggregate forming dye that has been functionalized occurs in the presence of a salt. In further embodiments, the salt is selected from the group consisting of KCl, NaCl, MgCl₂, CaCl₂, NiCl₂, MnCl₂, EuCl₃, TbCl₃, and a salt from a rare earth element.

Nanoscale MJ Conjugates

For certain applications, it may be advantageous to have nanoscale size MJs. Examples of such applications include applications that require the contrast agents to pass through biological barriers, for example, applications such as contrast-enhanced imaging of tumor lesions require nanosized particles. For such applications, the synthesis protocoal may be adapted to enable control over the size of MJ particles.

In some embodiments, the MJ is nanoscale, sub-micronscale, or microscale. In further embodiments, the MJ is nanoscale. In some embodiments, the MJ is 100 nm to 400 nm in size. In further embodiments, the MJ is 100 to 300 nm in size. In yet further embodiments, the MJ is 150 nm to 250 nm in size. In yet further embodiments, the MJ is 180 nm to 220 nm in size, for example about 200 nm in size.

Functionalization of MJs

Provided is an MJ conjugate of Formula I

MJ—E—D

Formula I

wherein

MJ is a modified J-aggregate;

E is a linker or a bond;

D is a functional moiety.

In some embodiments, the MJ comprises a J-aggregate forming dye.

In some embodiments, the MJ comprises a J-aggregate forming dye that has been functionalized.

In some embodiments, the MJ comprises a J-aggregate forming dye and a J-aggregate forming dye that has been functionalized.

In some embodiments, the J-aggregate forming dye has been functionalized with an azide group. In further embodiments, D has been attached to MJ by employing copper-free click chemistry with said azide group.

A schematic representation of how copper free click chemistry is carried out in an embodiment is shown below, wherein R is ICG:

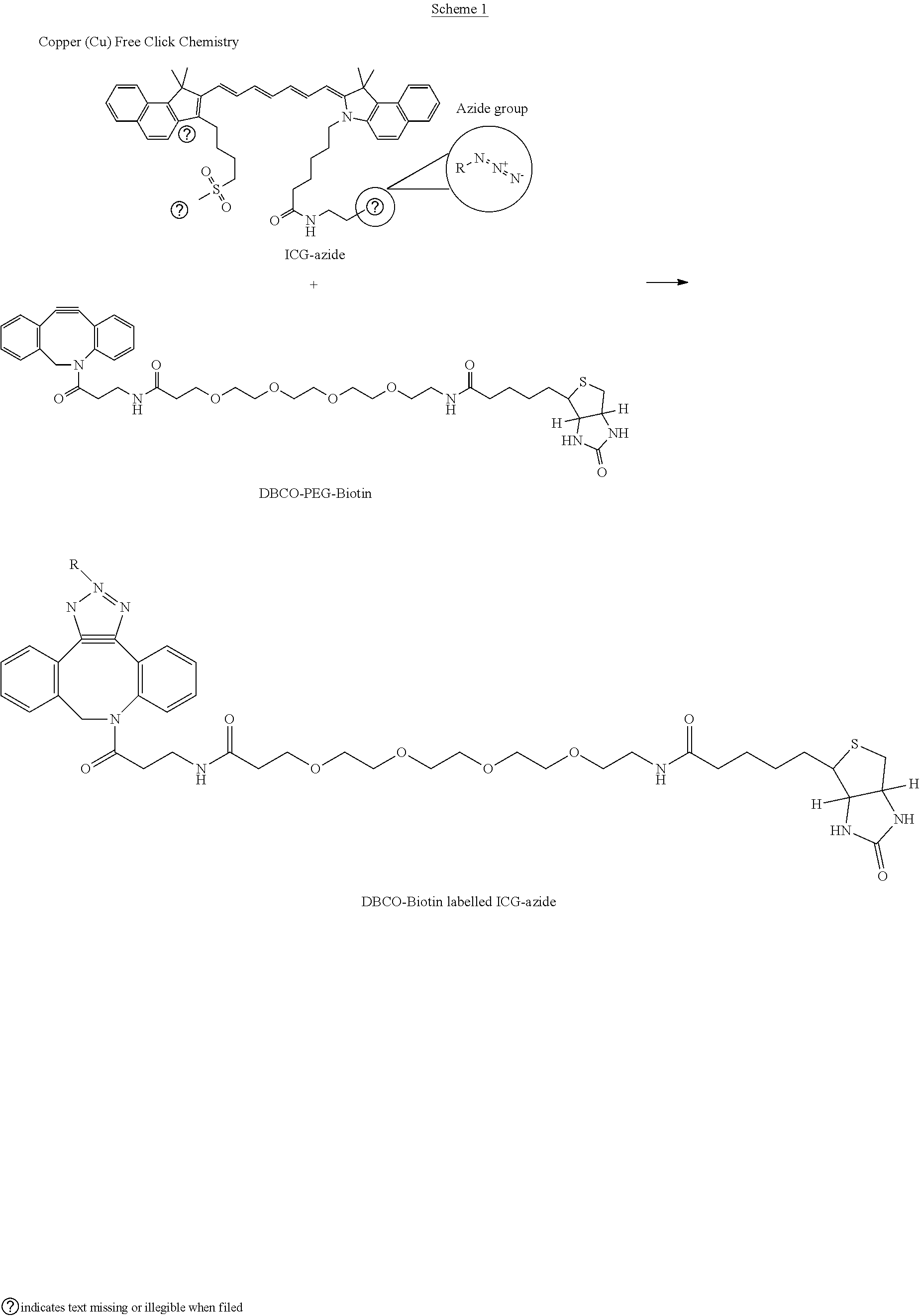

In some embodiments, the J-aggregate forming dye is a cyanine dye. In further embodiments, the J-aggregate forming dye is selected from the group consisting of indocyanine green (ICG), ICG-NHS, ICG-NH₂, pseudoisocyanine, merocyanine, bis(2,4,6-trihydroxyphenyl)squaraine, tetrtakis(4-sulfonatophenyl)-porphyrin, antimony(III)-phthalocyanine, copper phthalocyanine, perylene bismide, hypericin, subphtalocyanine, and combinations thereof.

In some embodiments, E is a linker comprising a copper-free click chemistry group. In further embodiments, E is a linker selected from the group consisting of DBCO-PEG-NHS, DBCO-PEG-Biotin, DBCO-PEG-Thiol, DBCO-PEG-NH₂, DBCO-PEG-Mal, Thiol-PEG-NHS, Thiol-PEG-Biotin, Thiol-PEG-NH₂, Mal-PEG-NHS, Mal-PEG-NH₂, Mal-PEG-Acrylate, Thiol-PEG-Acrylate, DBCO-PEG-Folate, and combinations thereof.

In some embodiments, D comprises streptavidin, biotin, RGD, or a derivative thereof. In some embodiments, D comprises a drug, a protein, a targeting protein, or an antibody. In further embodiments, the drug, protein, targeting protein or antibody is connected to E via a biotin-streptavidin interaction. In some embodiment, E comprises biotin and D comprises streptavidin. In some embodiments, E comprises streptavidin and D comprises biotin.

In some embodiments, the drug is a chemotherapeutic (e.g., Doxorubicin, Daunorobicin). In some embodiments, the drug is a steroid, a chemokine, a cytokine, a hormone, a therapeutic protein, an antimicrobial peptide, or a nucleic acid. In some embodiments, the nucleic acid is mRNA or siRNA. In some embodiments, the drug is bradykinin (BK).

In some embodiments, the protein is avidin, streptavidin, neutravidin, an enzyme, a signal peptide, a marker, a growth factor, a neurotransmitter, an antibody, an interferon, thrombin, an anticoagulant protein, a targeting peptide, or a cell penentrating peptide. In further embodiments, the antibody is a broad neutralizing antibody. In further embodiments, the anticoagulant protein is Protein C, Protein S or antithrombin.

In some embodiments, the targeting protein is a receptor, for example a chemokine receptor, a cytokine receptor, a hormone receptor, a growth factor receptor, a neurotransmitter receptor, or a cell adhesion receptor.

In some embodiments, the antibody is an antibody to a target tissue or an antibody to a target deep tissue. In some embodiments, the antibody is a monoclonal antibody, a polyclonal antibody, or a single domain antibody. In some embodiments, the antibody is anti-CTL4-4, anti-VCAM 1, anti-asialoglycoprotein receptor, or anti-VEGFR. In some embodiments, the monoclonal antibody is anti-CTL4-4, for example for targeting T lymphocytes. In some embodiments, the polyclonal antibody is anti-VCAM 1, for example for targeting kidney tissues. In some embodiments, the single domain antibody is anti-asialoglycoprotein receptor, for example for targeting liver tissues. In some embodiments, the single domain antibody is anti-VEGFR, for example a nanobody anti-VEGFR.

In some embodiments, the MJ conjugate is Bio-JAAZ, Strep-JAAZ or RGD-JAAZ, as further described in the Examples.

In some embodiments, the MJ conjugate is nanoscale, sub-micronscale, or microscale. In further embodiments, the MJ conjugate is nanoscale. In some embodiments, the MJ conjugate is 100 nm to 400 nm in size. In further embodiments, the MJ conjugate is 100 to 300 nm in size. In yet further embodiments, the MJ conjugate is 150 nm to 250 nm in size. In yet further embodiments, the MJ conjugate is 180 nm to 220 nm in size, for example about 200 nm in size.

Photoacoustic Imaging Using MJs or MJ Conjugates

Provided is a method of photoacoustic imaging at a target site comprising providing the MJ of any one of the embodiments described herein at the target site; and monitoring absorbance at the target site.

In some embodiments, the MJ absorbs in the near-infrared I (NIR-I) region (750-950 nm). In some embodiments, the MJ absorbs in the region of 450 to 950 nm. In further embodiments, the MJ absorbs in the near-infrared II (NIR-II) region of 1000 to 1700 nm.

In some embodiments, the MJ absorb incident light in the region of 450 to 950 nm and fluoresce to emit light in the near-infrared II (NIR-II) region of 1000 to 1700 nm.

In some embodiments, the target site is subjected to laser pulses at 1 to 100 pulses/s. In some embodiments, the target site is subjected to laser pulses at 1 to 2000 pulses/s, for example 10 pulses/s. In further embodiments, the target site is subjected to light pulses from light-emitting diodes at 1 to 2000 pulses/s.

In some embodiments, the target site is a tissue. Tissues include, but are not limited to, vascular tissue, placental vasculature, skin, brain, breast, ovarian, testicular, prostate, lung, colon, liver and all the hepatocellular system, spleen, uterine cervix, and kidney.

In further embodiments, the target site is a deep tissue. Deep tissues include, but are not limited to, lymph node, gastrointestinal tract, bladder, pancreas, colon, kidney, liver, and brain.

Provided is a method of photoacoustic imaging at a target site comprising providing the MJ conjugate of any one of the embodiments described herein at the target site; and monitoring absorbance at the target site.

In some embodiments, the MJ conjugate is Bio-JAAZ, Strep-JAAZ or RGD-JAAZ, as further described in the Examples.

In some embodiments, the MJ conjugate absorbs in the near-infrared I (NIR-I) region (620-950 nm). In some embodiments, the MJ absorbs in the region of 450 to 950 nm. In further embodiments, the MJ absorbs in the near-infrared II (NIR-II) region of 1000 to 1700 nm.

In some embodiments, the MJ conjugate absorbs incident light in the region of 450 to 950 nm and fluoresce to emit light in the near-infrared II (NIR-II) region of 1000 to 1700 nm.

In some embodiments, the target site is subjected to laser pulses at 1 to 100 pulses/s. In some embodiments, the target site is subjected to laser pulses at 1 to 2000 pulses/s, for example 10 pulses/s. In further embodiments, the target site is subjected to light pulses from light-emitting diodes at 1 to 2000 pulses/s.

In some embodiments, the target site is a tissue. Tissues include, but are not limited to, vascular tissue, placental vasculature, skin, brain, breast, ovarian, testicular, prostate, lung, colon, liver and all the hepatocellular system, spleen, uterine cervix, and kidney.

In further embodiments, the target site is a deep tissue. Deep tissues include, but are not limited to, lymph node, gastrointestinal tract, bladder, pancreas, colon, kidney, liver, and brain.

Use of the MJ or MJ conjugates described herein may reduce the rate of secondary tumor resection surgeries. It may allow conservation of more healthy tissue. This may improve patient outcome.

Tumor Margin Staining Using MJs or MJ Conjugates

In some embodiments, MJ or MJ conjugate is formulated in a water-based sprayable form (solvents can be water, PBS or other water-based physiological solution) that can be used for staining tumor margin directly after surgical resection of the tumor from the patient. The MJ or MJ conjugate may be micro or nanoscale.

In some embodiments, the MJ or MJ conjugate helps detect the presence of tumor cells near to the surface of the resected tumors via specific targeting moieties (adapted to the tumor targeted) that enable tumor-cell specific labelling.

Using photoacoustic imaging, presence of tumor cells close to the margin of the resected tumor is evaluated.

Near-Infrared II (NIR-II) Imaging

NIR-II is the second NIR optical window that encompasses wavelengths ranging from 1000-1700 nm, respectively. Using these longer wavelengths to perform fluorescence imaging reduces the light scattering and autofluorescence typically observed in biological tissues and allows for deeper imaging with improved signal-to-background ratio. MJs and MJ conjugates have a strong and characteristic fluorescence emission signal detectable in the NIR-II windows when excited at wavelengths of 808 (that correspond to ICG absorption peak) and 890 nm (that correspond to ICG aggregates absorption peak).

In some embodiments, MJ or MJ conjugate is used to perform targeted deep in vivo imaging using dual imaging modality, namely photoacoustic and fluorescence imaging.

Photothermal Therapy

MJ or MJ conjugate is used to perform photothermal therapy that can be used to kill cancer cells locally. In some embodiments, the MJ or MJ conjugate is free. In some embodiments, the MJ or MJ conjugate is encapsulated in a liposome. In some embodiments, the MJ or MJ conjugate performs photothermal therapy using NIR excitation (e.g., 808 nm laser-irradiation for 100 to 300 s). By including tumor targeting moieties in the MJ or MJ conjugate, photothermal destruction of tumors may be conducted.

### EXAMPLES

Materials and Methods

Materials:

Indocyanine Green (ICG) and ICG-Azide were purchased from AAT Bioquest (Pleasanton, Calif., USA). Dibenzylcyclooctyne-PEG4-Biotin (DBCO-PEG4-Biotin) was acquired from Jena Biosciences (Jena, Germany). Streptavidin and Cyclo-Arg-Gly-Asp-D-Phe-Lys(Biotin) (c[RGDfK(Biotin)]) (SEQ ID NO: 1) were purchased from Vivitide (Louisville, Ky., USA). Filtration unit Amicon Ultra-0.5 centrifugal filter, potassium chloride (KCl) and Bicinchoninic Acid (BCA) Kit for protein determination were acquired from Sigma-Aldrich (St. Louis, Mo., USA). Triton X-100, Tween-20, Dimethylsulfoxide (DMSO) and glycerol were purchased from Fischer Scientific (Waltham, Mass., USA). Phosphate-Buffered Saline (PBS, 10X solution at pH 7.4) was acquired from ThermoScientific (Waltham, Mass., USA). Agarose, high strength (1200 g/cm²) was purchased from Agarose Unlimited (Alachua, Fla., USA). Agarose, low melt (>500 g/cm²) was purchased from IBI Scientific (Dubuque, Iowa, USA). Molecular biology grade water was purchased from Quality Biological (Gaithersburg, Md., USA). Low-volume quartz glass cuvettes (ZEN2112) and DTS-1070 folded capillary zeta cells were purchased from Malvern Analytical (Malvern, U.K.), 1X Dulbecco's Modified Eagle's Medium was purchased from Corning Inc (Corning, N.Y., USA) and Fetal Bovine Serum (FBS) were purchased from VWR Life Sciences (Radnor, Pa., USA). India Ink and whole sheep blood containing anticoagulant were purchased from Hardy Diagnostics (Santa Maria, Calif., USA). Eagle's Minimum Essential Media (EMEM) and Ethylenediaminetetraacetic acid (EDTA) were purchased from ATCC (Manassas, Va., USA). Penicillin was purchased from Millipore Sigma (Burlington, Mass., USA).

Methods:

Preparation of ICG-JA and JAAZ particles: ICG stock solutions were prepared at a concentration of 5 mg/ml in molecular biology grade water and stored at −20° C. ICG-azide stock solution was made at 10 mg/ml in DMSO and stored at −20° C. To assemble the ICG-JA, ICG dye was prepared at 1 mM in molecular biology grade water complemented with 20 mM of KCl and incubated at 60° C. for 20 hours. For each molar ratio of ICG-azide:ICG solution, 1:20, 1:10, 1:5 and 1:3, a solution of the corresponding molarity of ICG and ICG-azide was prepared. For JAAZ microparticles, the different solutions of ICG and the corresponding molarity of ICG-azide was incubated in 20 mM KCl for 20 hours at 60° C. to a final concentration of 1 mM total dye. Unless otherwise specified, JAAZ microparticle samples were prepared with 1:10 ICG-azide:ICG molar ratio to a final concentration of 1 mM total dye.

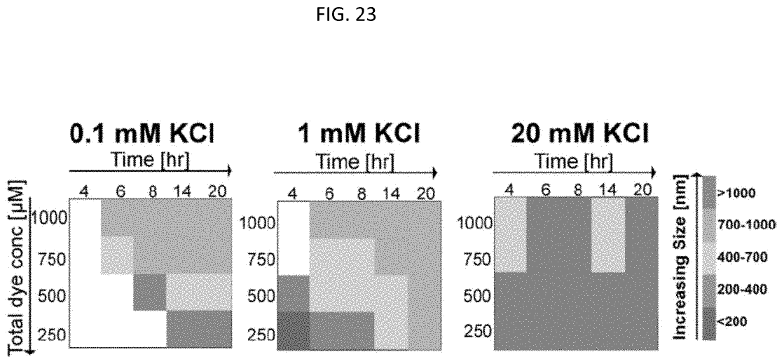

Preparation of N-JAAZ particles: For JAAZ particles in the 600-700 nm range, the reaction solution of 1:10 ICG-azide:ICG molar ratio was incubated in 1 mM KCl for 20 hours at the same temperature as mentioned above to a final concentration of 1 mM total dye. To make nanosized JAAZ particles of size ˜200 nm, 250 μM total dye solution was incubated in 0.1 mM KCl for 14 hours at 60° C. A complete summary of the total dye concentration at 1:10 ICG-azide:ICG molar ratio, KCl molarity and time required for the formation of JAAZ particles is given in FIG. 23.

Filtration of JA: Unless otherwise specified, prepared ICG-JA and JAAZ samples were filtered using 0.5 ml Amicon centrifugal filters (100 kDa cutoff) to remove the non-aggregated free monomers or dimers of ICG dye, if present. Filtration was carried out by centrifugation at 4000 g for 3 minutes at room temperature. Three cycles of filtration in molecular biology water at a total volume of 0.5 ml were used to ensure complete removal of unreacted dye. The percentage of samples recovered after filtration was calculated by:

% recovery=[(A894_{(BF)}−A894_{(AF)}/A894_{(BF)}]*100

where A894(BF) is the absorbance intensity at 894 nm before filtration (BF) and, A894_{(AF)} is the 459 absorbance intensity after filtration.

Characterization of JA

Standard Curve: The amount of free dye in all J-aggregate samples was determined by incubating the formed J-aggregate particles in 1% Triton X-100 for 10 minutes at 37° C. and scanning the samples from 400-990 nm. A standard curve of a 1:10 molar solution of ICG-azide:ICG dye was created to determine the free dye concentration in the dissociated J-aggregate samples.

Absorbance measurements: To confirm the presence of J-aggregates, the samples were scanned from 440 nm to 990 nm with 2 nm step size using a PowerWavex microplate spectrophotometer by Bio-Tek Instruments Inc. The excitation and emission wavelengths of free ICG dye are 780 and 800 nm respectively. The emission wavelength of ICG-JA was determined at 895 nm. Unless specified, all readings were taken at a free dye concentration of 25 μM determined using the standard curve.

Fluorescence measurements: Fluorescence intensity of different concentrations of free ICG dye and J-aggregates was recorded using a Tecan Safire²™ (Mannendorf, Switzerland) fluorescence microplate reader. Both free ICG dye and J-aggregate particles were excited at 720 nm with an emission at 770 nm. The fluorescence emission spectrum was recorded from 770 to 850 nm with a step size of 2 nm.

Dynamic Light Scattering (DLS) measurements: The hydrodynamic diameter of the formed ICG-JA and JAAZ particles was measured using a Malvern Zetasizer Nano ZS™ (Malvern, United Kingdom) instrument. The size measurements were taken using low-volume quartz glass cuvettes at 25 μM of free dye concentration. The zeta potential (surface charge) of all samples was determined using the Malvern Zetasizer Nano ZS™ using DTS-1070 folded capillary zeta cells. The measurements for Zeta potential were taken at a dye concentration of 15 μM and a volume of 700 μl in 0.1X PBS at pH 7.4.

Gel Electrophoresis: A 2% (w/v, in MilliQ water) agarose gel was prepared using high strength agarose. The agarose gel was casted in a square gel caster. A square window of 6×6 cm was cut in the 2% gel using a blade. Low melting agarose of 0.05% (w/v, in MilliQ water) was poured in the cut window containing well-combs and the gel polymerized at 4° C. overnight. 30 μl of 500 μM JAAZ and N-JAAZ samples were mixed with an equal volume of glycerol and loaded into the wells. The gel was run at 120 V for 10 minutes.

Scanning Electron Microscopy: 5 μl of the JAAZ samples prepared at dye concentration of 1 mM with 20 mM KCl or 5 μl of the N-JAAZ (183 nm) samples prepared at dye concentration of 250 μM with 0.1 mM KCl incubated for 20 or 14 hours at 60° C., respectively, were deposited on a clean mica surface and vacuum dried overnight. SEM images were collected using Jeol JSM-IT500HR InTouchScope™ (Tokyo, Japan) at an accelerating voltage of 15 kV and a working distance of 10 mm. EDX spectra were collected using Octane Elect EDS System at an accelerating voltage of 15 kV and a resolution of 127 eV. ImageJ (63) was used to determine the size distribution of the particles in the obtained SEM images.

Streptavidin functionalization of JAAZ particles: 500 μM of JAAZ or N-JAAZ particles with 1:10 ICG-azide:ICG molar ratio were labelled with DBCO-PEG4-Biotin at a concentration 10 times greater than the estimated number of azide groups available (Table 2).

TABLE 2
Samples and their attached functional groups/biomolecules

| | Composition and Functional Groups Attached | | | | |
|---|---|---|---|---|---|
| | | ICG- | DBCO- | Strepta- | RGD- |
| Sample | ICG | N₃ | Biotin | vidin | Biotin |
| Name | (mM) | (mM) | (Molar ratio with respect to ICG-N₃) | | |
| JA | 1 | — | — | — | — |
| JAAZ | 1 | 0.1 | — | — | — |
| Bio-JAAZ | 1 | 0.1 | 10X | — | — |
| Strep-JAAZ | 1 | 0.1 | 10X | 10X | — |
| RGD-JAAZ | 1 | 0.1 | 10X | 10X | 10X |

The azide group on the J-aggregates reacts with the DBCO end of DBCO-PEG4-Biotin in an aqueous buffer free of metal catalysts (water or 1X PBS) and this reaction is known as copper-free click chemistry. The samples were incubated and mixed in an incubator shaker overnight at room temperature protected from light. To remove excess DBCO-Biotin, the samples were filtered using the same procedure mentioned in the filtration section. These biotin functionalized samples (Bio-JAAZ) were diluted to 100 μM of free dye concentration and treated with 10 mg/ml streptavidin at 5X concentration of the available biotin sites (Strep-JAAZ). The samples were kept at 4° C. for two hours to ensure attachment of streptavidin to the available biotin sites. Excess streptavidin was removed by filtration using 100 kDa mini-centrifugal filters. The samples were filtered by centrifugation at 4000 g for three minutes. Four cycles of filtration were carried out to ensure complete removal of free streptavidin. The absorbance spectra of all samples were tabulated. The Strep-JAAZ samples were labelled with c[RGDfK(Biotin)] at 10X molar concentration and incubated for two hours at 4° C. Excess c[RGDfK(Biotin)] was removed by filtration using the same procedure as mentioned before. The labelled samples were named as RGD-JAAZ. All Bio-JAAZ, Strep-JAAZ and RGD-JAAZ samples were characterized using the same procedure as mentioned in the characterization section.

Determination of amount of streptavidin attached: All Strep-JAAZ samples after filtration were diluted to 5 μM of free dye concentration. BCA working reagent was made following the instructions provide by the supplier. 25 μl of 5 μM samples are incubated in 200 μl of BCA working solution and the amount of streptavidin attached is calculated using a BSA standard curve. Bio-JAAZ samples are used as control in every experiment.

Photoacoustic Measurement: The 96-well plate was secured to the bottom of a plastic water reservoir. The reservoir was filled with deionized water, completely submerging the sample. For comparison of smaller volumes, 2-inch segments of 24-gauge PTFE tubing (˜100 μL) was filled with various samples. The tubes were placed in a clear acrylic chamber submerging the samples in deionized water. A single element piezoelectric 35 MHz or 5 MHz focused transducer (V326, Olympus, Mass., USA) was fixed at 12 mm or 45 mm above each sample with their functional element submerged in deionized water. Optimization of positioning and photoacoustic signal from each sample was done though movement of a fine x-y axis stage, moving above each sample with a fixed transducer and pulsed laser. Excitation of the sample was done obliquely by a wavelength-tunable (690-950 nm) pulsed laser designed for photoacoustic imaging (Phocus Mobile, Opotek) at the peak absorption wavelength for the JAAZ (895 nm) for magnitude comparison. The photoacoustic signal recorded from the transducer is sent to a 20/40db amplifier (HVA-200M, Femto) from there the signal is recorded through a lock-in amplifier (Zurich Instruments, Zurich, Switzerland) triggered on each laser pulse using a photodiode placed near the laser source. For each measurement, a 10 second recording (100 waveforms) was taken for each sample (or at each wavelength, for PA spectrum generation). The absolute amplitude of each waveform was calculated, averaged, and normalized to the signal recorded from India ink or whole sheep's blood containing anticoagulant.

Photoacoustic Imaging

Cell culture: Hela cells were cultured in EMEM augmented with 10% Fetal Bovine Serum, and 1% Penicillin and kept in an incubator at 37° C. and 5% CO₂. HeLa cells were seeded using 0.25% (w/v) Trypsin-0.53 mM EDTA solution to glass slides at a concentration of 1.0×10⁵ cells/mL.

Cell and Phantom Imaging: Plain glass slides cultured with Hela cells were stained with 10 μM JAAZ or RGD-JAAZ for 20 minutes in an incubator at 37° C. and 5% CO₂. Short segments of 24-gauge PTFE tubing (˜100 μL) were filled with 10 μM RGD-JAAZ and arranged on a glass slide. The slides were then placed under a single element piezoelectric 35 MHz (or 15 MHz) focused transducer fixed at 12 mm (or 2 inches) above the surface of the slide. A wavelength-tunable (690-950 nm) pulsed laser designed for photoacoustic imaging (Phocus Mobile, Opotek) was obliquely shown over the slide. The peak absorption wavelength for the JAAZ (895 nm) was used to excite the sample and the photoacoustic signal recorded from the transducer was sent to a 20/40 dB amplifier (HVA-200M, Femto) from there the signal was recorded through a lock-in amplifier (Zurich Instruments, Zurich, Switzerland) triggered on each laser pulse using a photodiode placed near the laser source. The sample was then raster scanned through movement of the stage at 100 μm steps (with fixed laser source and transducer) mounted on motorized translation stages (Thorlabs). Absolute signal magnitude was then plotted in 2D space to create the image. A simple Matlab image gaussian filter was then used to smooth the image.

Statistical Analysis: All bar graphs are expressed as the mean±standard deviation. Statistical analyses were performed using the unpaired, two-tailed Student's t-test and One-Way Analysis of Variance (ANOVA) with a Tukey post-hoc test using the software GraphPad Prism 9.0.2. Data was deemed statistically significant if the p value was <0.05. Absolute PA signal amplitude is defined as the sum of the absolute minimum and maximum peak PA signal.

### Example 1: Formation of Azide-Modified J-Aggregates (JAAZ)

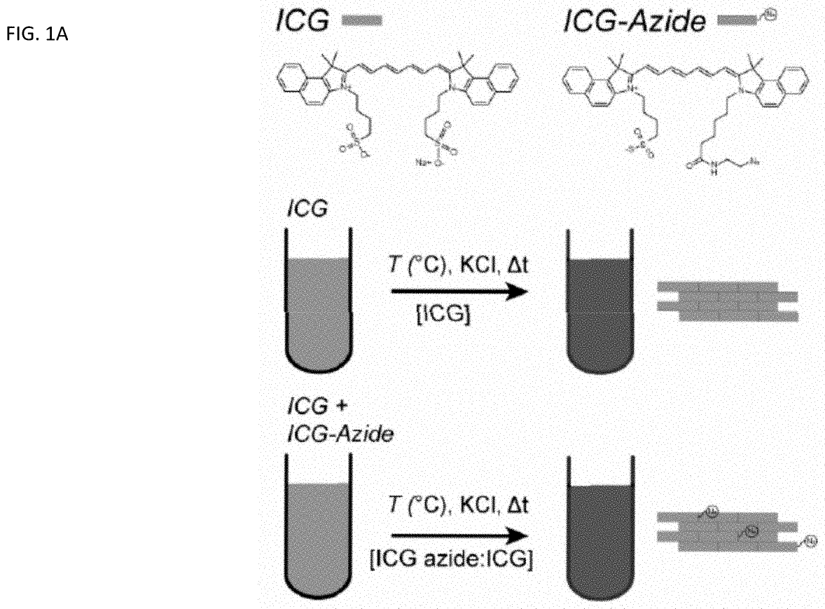

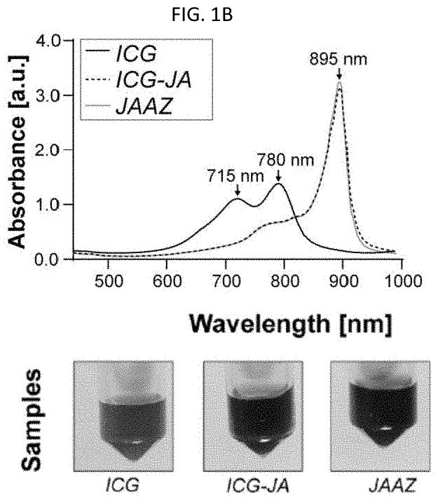

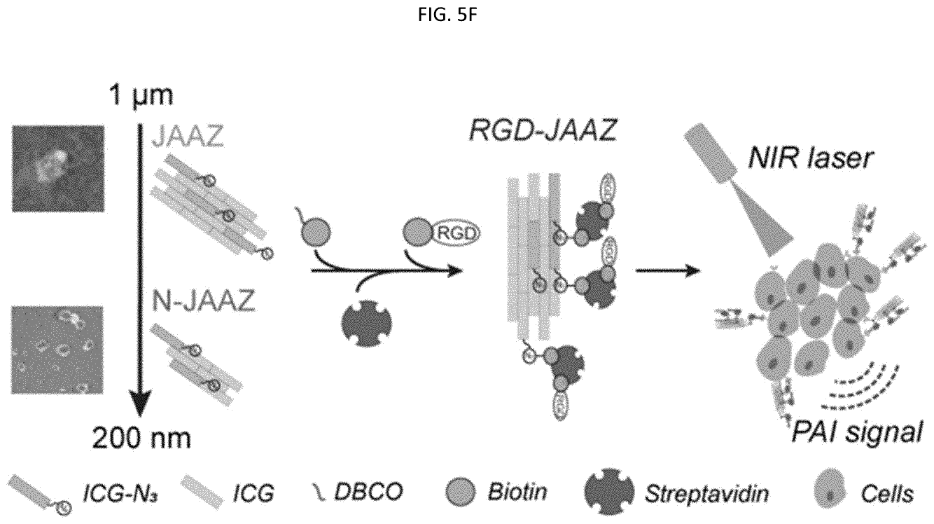

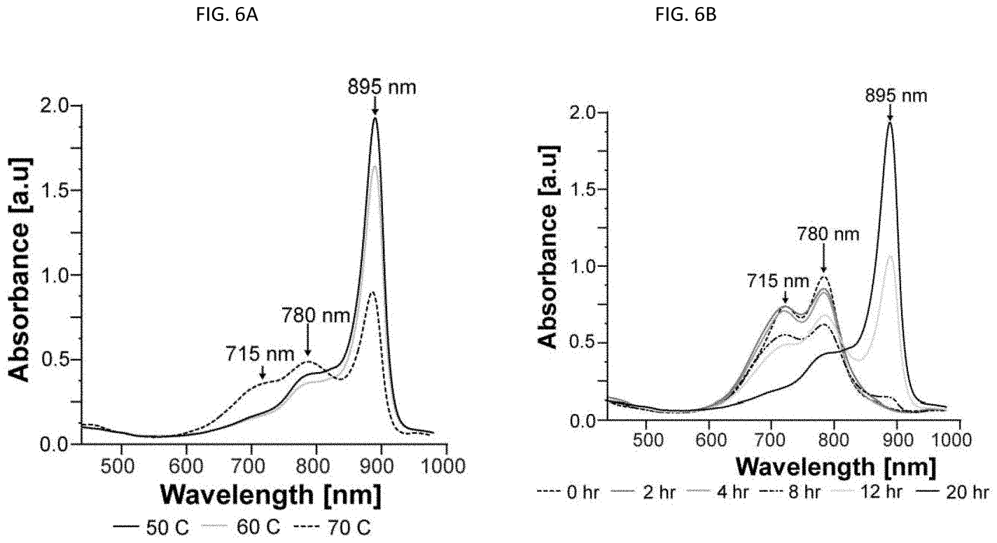

To assemble functionalized ICG-JA that could be further modified with targeting moieties using simple and robust copper-free click chemistry, J-aggregates were prepared using a combination of ICG and ICG-azide dyes (FIG. 1A). Prior to assembling azide-modified J-aggregates (JAAZ), ICG-JA formation was verified and characterized by incubating ICG dye at an initial concentration of 1 mM in water for 20 hours at 60° C., using a method previously described by Liu et al (45). Using UV-visible-NIR spectroscopy, formation of ICG-JA was monitored over multiple time points and confirmed by the red-shifted absorption peak at 895 nm and the disappearance of both the monomer and the dimer peaks (780 and 715 nm, respectively). (FIG. 1B and FIGS. 6A-6B).

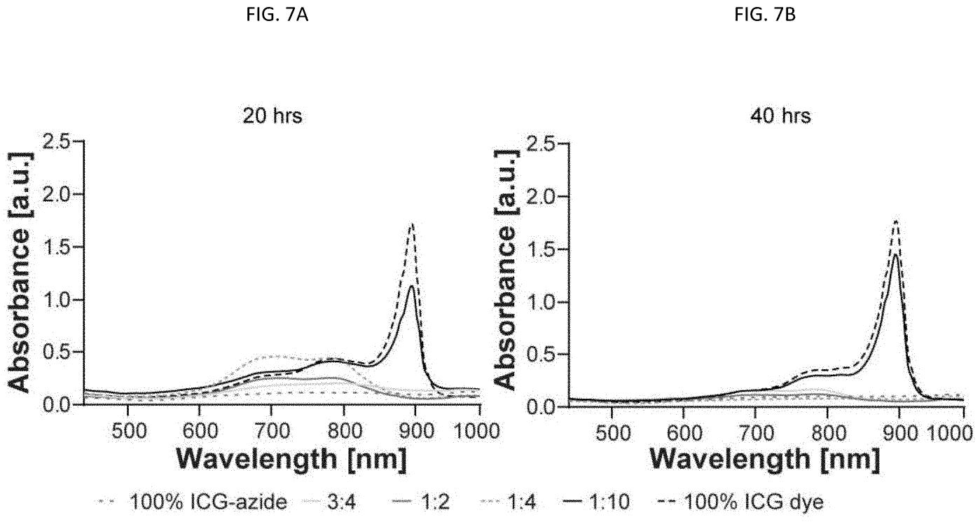

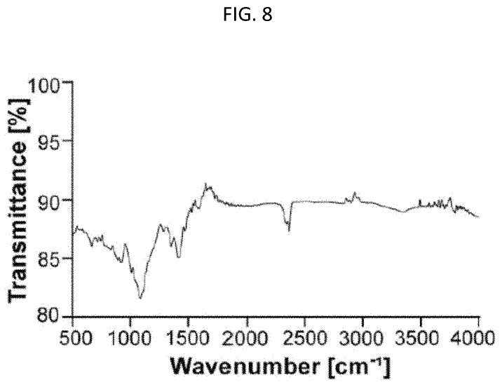

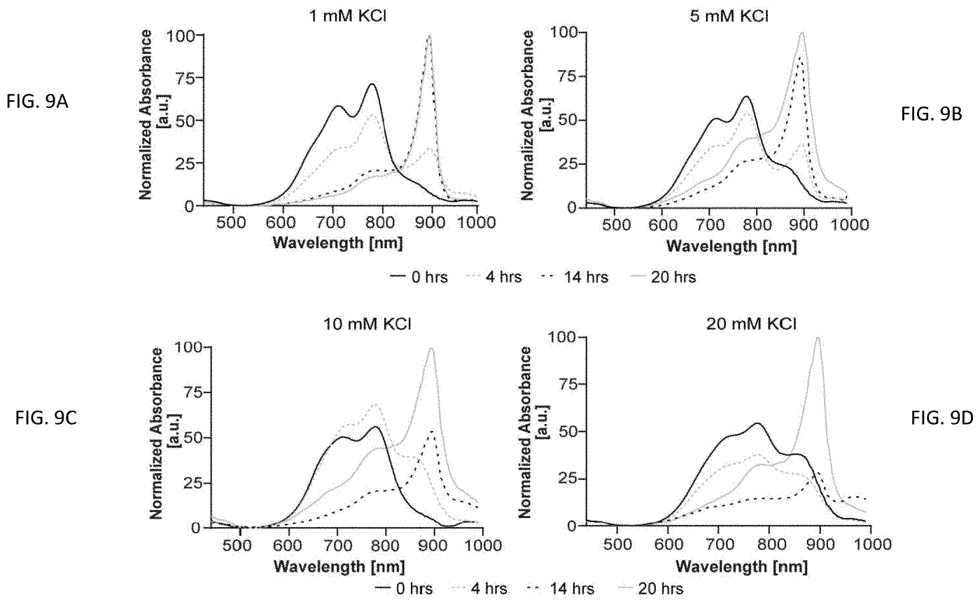

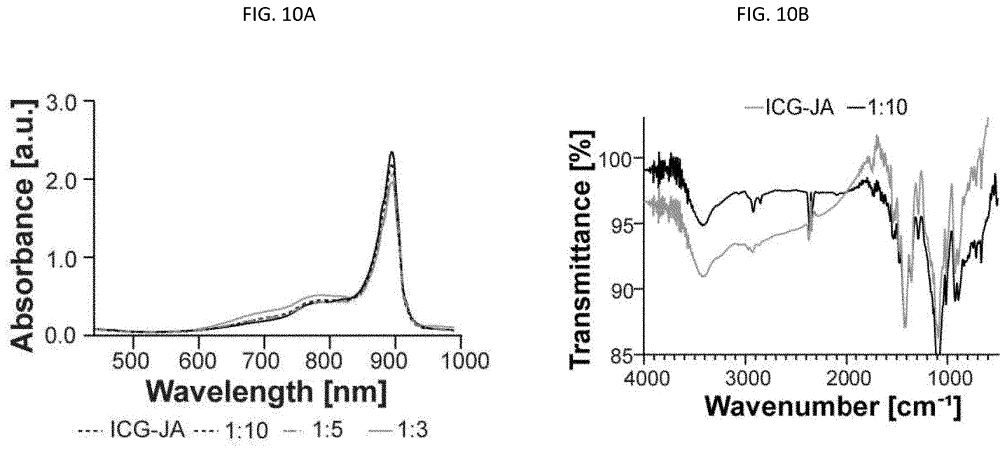

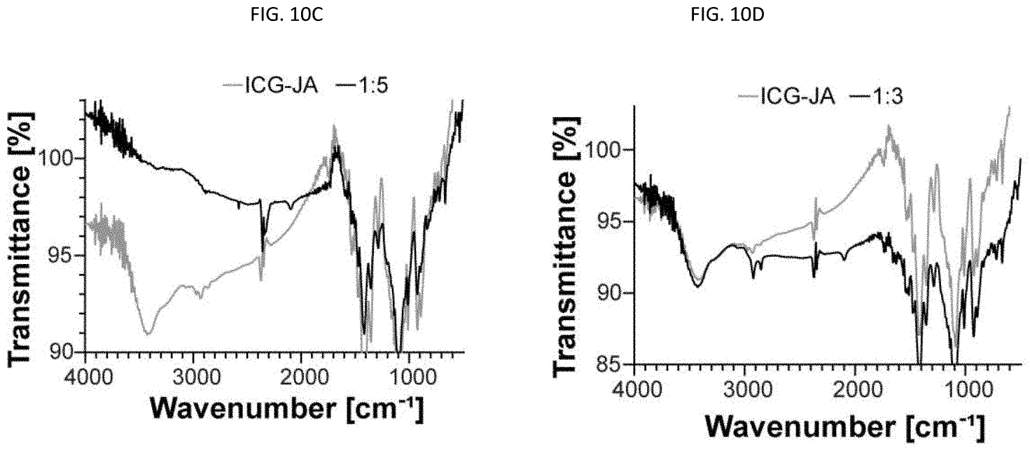

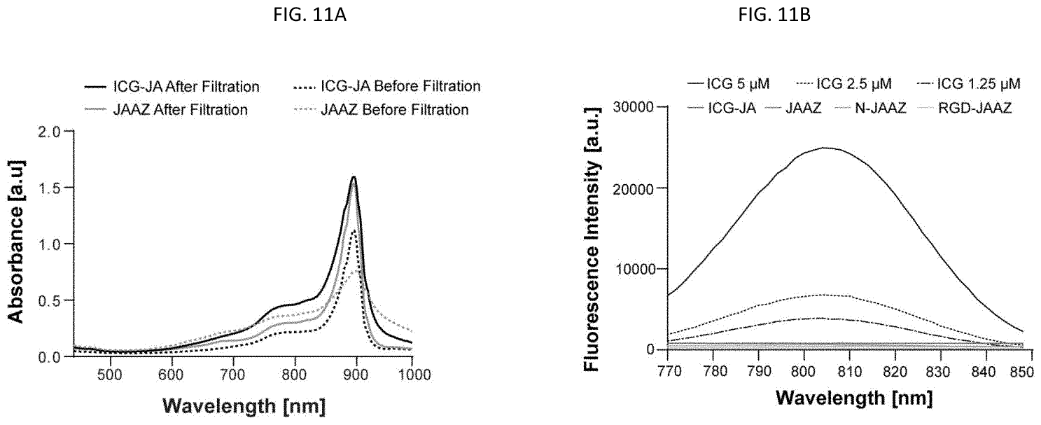

The same protocol was applied to assemble JAAZ particles. Mixtures of ICG dye and azide-modified ICG dye were incubated at different molar ratios of ICG-azide to ICG. After incubation for 20 and 40 hours at 60° C. in water, the typical J-aggregate peak was not observed at 895 nm (FIGS. 7A and 7B, respectively) for 100% ICG-azide dye or for ICG-azide:ICG molar ratios of 3:4, 1:2 and. 1:4. In contrast, the 1:10 ICG-azide:ICG solution in water did form J-aggregates with peak absorption at 895 nm (FIG. 7). However, the Fourier-transform Infrared (FT-IR) spectrum did not show a peak between 2141-2100 cm⁻¹ (FIG. 8), which is a characteristic range for the azide group due to the strong asymmetric N=N=N stretching (52), indicating that the formed J-aggregates do not contain ICG-azide. To promote the incorporation of ICG-azide into the aggregates, the protocol was modified to include salts (53) which are known to influence aggregate formation (53, 54). Specifically, monovalent metal cations like K⁺ are known to facilitate J-aggregate formation of anionic cyanine dyes such as ICG and pseudoisocyanine (PIC) due to ionic interactions between cations and the dye molecules (55). Thus, different concentrations of potassium chloride (KCl) (1, 5, 10 and 20 mM) were tested with 1:10 ICG-azide:ICG molar ratio solutions. After 20 hours of incubation at 60° C., the J-aggregates prepared with each KCl concentration showed an absorption peak at 895 nm (FIG. 9). JAAZ particles were also synthesized with different molar ratios of ICG-azide to ICG (1:10, 1:5 and 1:3) using 20 mM KCl. The formation of J-aggregates for the different ratios were confirmed by the presence of the 895 nm peak in the UV-Vis-NIR spectra (FIG. 10A). FT-IR spectra of all synthesized JA particles with KCl showed a peak at 2100 cm⁻¹ due to incorporated azide groups, which was absent from the control ICG-JA (FIGS. 10B-D). Therefore, using the modified protocol with KCl, ICG-JA and JAAZ particles were able to be formed (FIGS. 1A-1B). The formation of J-aggregates in both cases was confirmed by observation of a sharp absorption peak at 895 nm and a decrease of both monomer and dimer ICG peaks in the absorbance spectrum (FIG. 1B). The solutions of both ICG-JA and JAAZ obtained after incubation became slightly darker green than the free ICG dye as expected for formation of J-aggregates (FIG. 1B). Excess free dye and residual salt were efficiently removed from the prepared ICG-JA and JAAZ using spin column filtration (FIG. 11A) before further modification of the J-aggregates. Successful formation of J-aggregates is also characterized by the absence of fluorescence (46) as seen in FIG. 11B. Free ICG dye has a recordable fluorescence intensity at concentrations of 5, 2.5 and 1.25 μM, whereas both ICG-JA and JAAZ have negligible fluorescence (FIG. 11B).

### Example 2: Characterization and Stability of JAAZ Microparticles

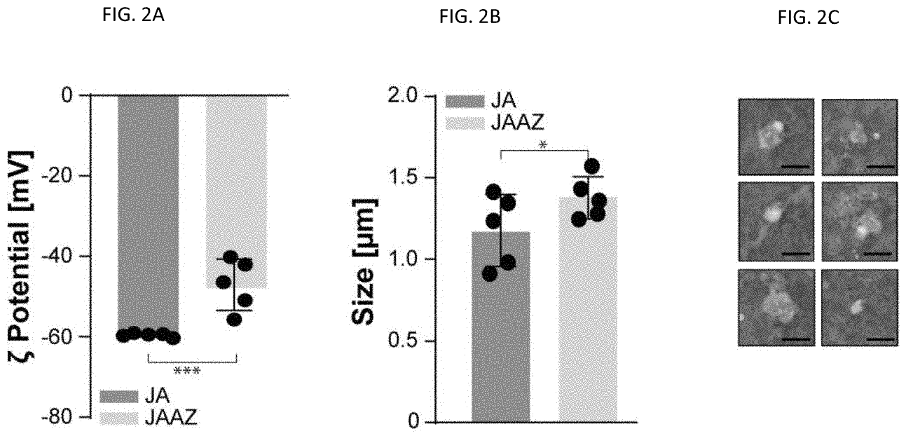

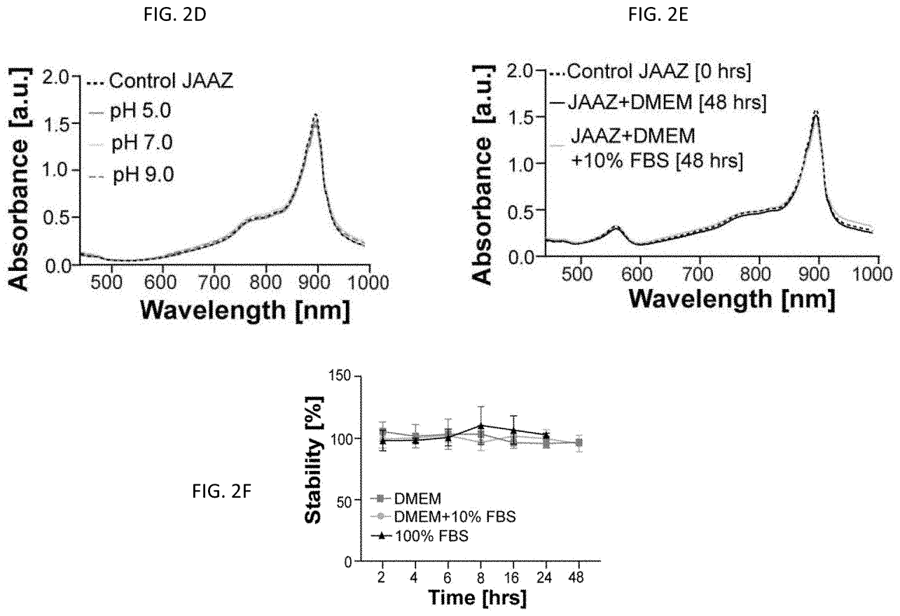

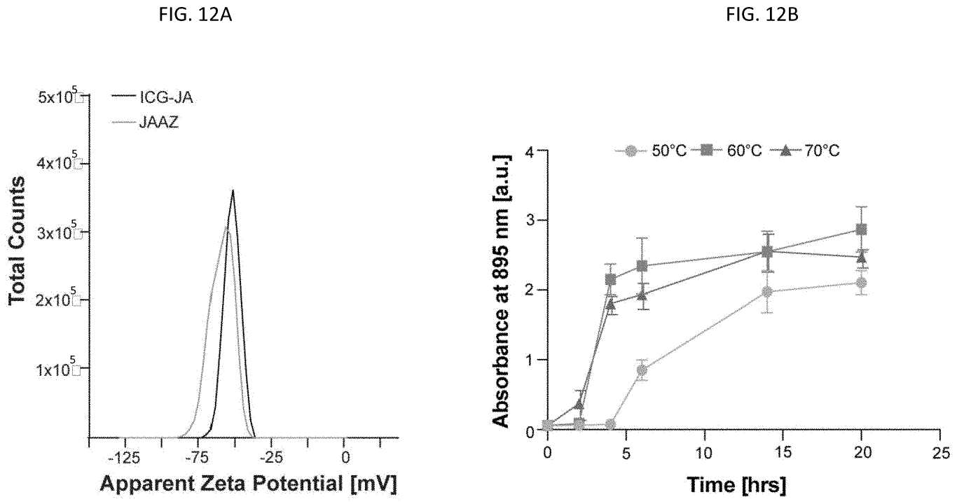

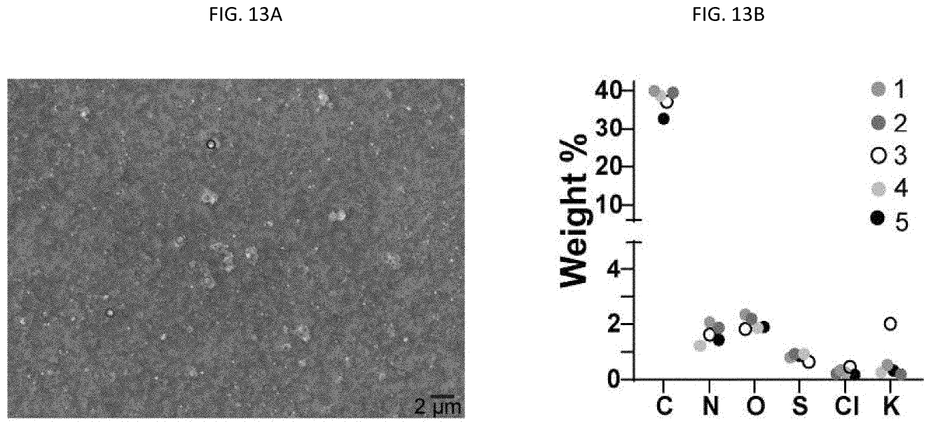

After confirming formation of JAAZ particles, the presence and accessibility of the azide group was examined using measurements of size and surface charge (zeta potential). The surface zeta (ζ) potential of both ICG-JA and JAAZ was first measured, knowing that the difference in charge between the ICG-azide and the regular ICG dye should modify the overall surface charge of the JAAZ particles. The average ζ potential of the JAAZ particles was measured at −50.32 mV, as opposed to the ζ potential of ICG-JA at −60.11 mV (FIG. 2A). The slightly less negative zeta potential of the JAAZ particles indicates the presence of multiple azide groups on their surfaces. The surface charge distribution is homogeneous with similarly charged J-aggregates in the solution which would confirm the presence of one type of J-aggregate population (FIG. 12A). Using dynamic light scattering (DLS) the average hydrodynamic diameter of the formed ICG-JA and JAAZ particles was measured at 1.17±0.26 and 1.34±0.18 μm, respectively (FIG. 2B). The slightly larger size of JAAZ particles could be attributed to a less densely packed dye arrangement due to the presence of the azide group and the difference in the dye charges (FIG. 2). The size distribution of JAAZ particles was more monodisperse than that of ICG-JA with a polydispersity index (PDI) of 0.37 for JAAZ and 0.61 for ICG-JA. Scanning electron microscopy (SEM) images of JAAZ particles exhibited uniform morphology with an apparent average diameter close to 1.1 μm (FIG. 2C and FIG. 13), in agreement with the DLS data. Energy-dispersive X-ray (EDX) analysis (FIG. 13B) confirmed the elemental composition of JAAZ microparticles.

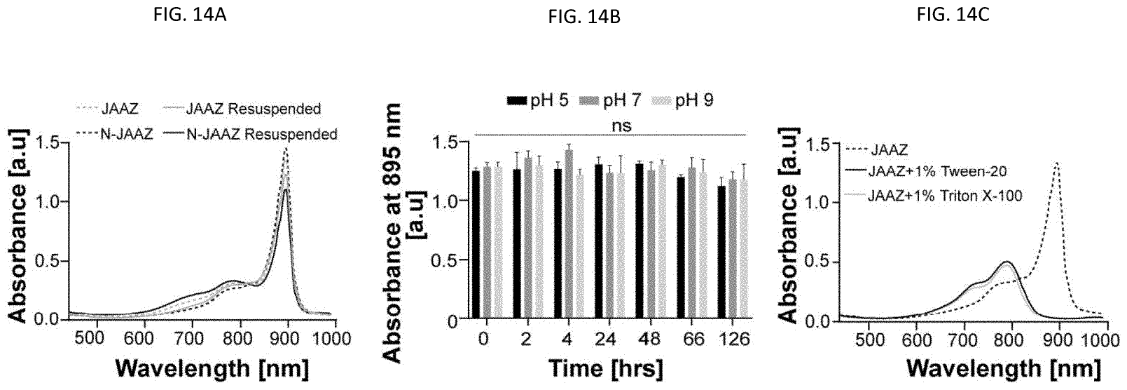

Importantly, the synthesized JAAZ microparticles could be lyophilized and resuspended in water with no significant change in the absorption spectra (FIG. 14A). To further determine the stability of the microparticles, the size and ζ potential of the rehydrated samples were recorded after resuspension (Table 1). The size of the JAAZ particles before drying and after resuspension in water were not significantly different (Table 1), suggesting JAAZ microparticles can be stably stored in lyophilized form at room temperature protected from light. They are easily dispersed in different media, such as 1x phosphate buffered saline (PBS) at different pH, Dulbecco's Modified Eagle Medium (DMEM), and DMEM+10% fetal bovine serum (FBS, mimicking biological conditions). When incubated in 1×PBS at pH 5.0, 7.0, and 9.0 at 37° C. for 126 hours, no significant changes were observed in the absorption spectra (FIG. 2D). There was no statistical difference in the maximum absorbance at 895 nm for 126 hours at all three pH conditions (FIG. 14B), indicating the stability of JAAZ microparticles under physiological conditions. JAAZ particles were also stable in both DMEM and DMEM supplemented with 10% FBS for 48 hours, as evidenced by the lack of difference in the absorbance intensity at 895 nm (FIG. 2E), an improvement from the stability of ICG-JA observed in a previous study (45). Since J-aggregates are formed due to non-covalent interactions between the dye molecules, JAAZ particles can be easily dissociated into dye monomer and dimer in detergents (Triton X-100, Tween-20) as seen in FIG. 14C.

TABLE 1
Size and charge (zeta potential) of JAAZ and N-JAAZ before
and after vacuum drying and resuspension in water.

| | Size (nm) | | Zeta Potential (mV) | |
|---|---|---|---|---|
| Sample | Before Drying | After Drying | Before Drying | After Drying |
| JAAZ | 1,083 ± 52 | 1,030 ± 256 | −52.1 ± 2 | −55.0 ± 5 |
| Nanosized | 281.8 ± 37 | 360.9 ± 38 | −54.7 ± 1 | −57.1 ± 3 |
| JAAZ | | | | |

Sample size is n = 3 for DLS measurement; p = 0.8470 = ns for JAAZ; p = 0.4622 = ns for N-JAAZ (Statistics are for DLS measurement); n = 2 for zeta potential measurement.

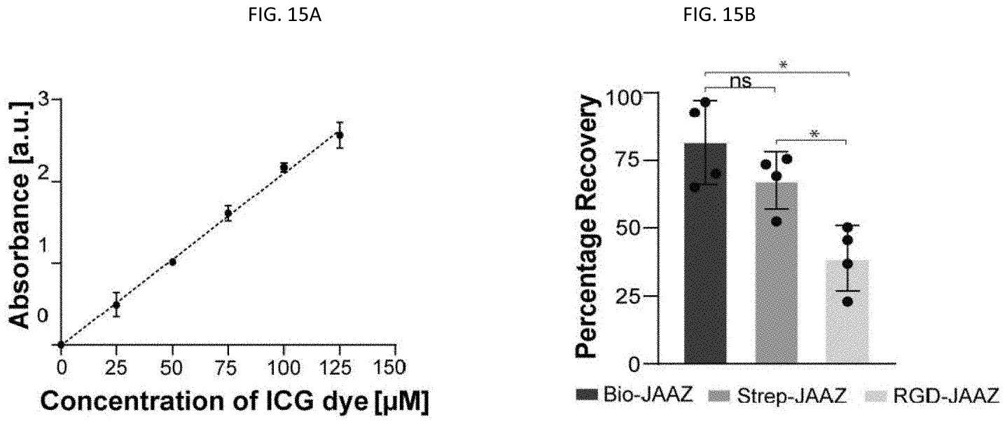

Before further modification of JAAZ particles, the free dye concentration of ICG-201 JA and JAAZ particles was determined, to quantify the incorporation of dye into these J-aggregates and correlate the absorbance with the total concentration of dye. The filtered JAAZ particles were dissolved into free dye molecules using 1% Triton X-100 (TX-100) and the concentration of free dye was determined from a standard curve of ICG dye dispersed in the same TX-100 concentration (FIG. 15A). When JAAZ and ICG-JA particles were dissolved, the total free dye concentration measured was similar for both J-aggregates, which indicate that presence of ICG-azide does not reduce the efficiency of J-aggregate formation.

### Example 3: Functionalization of JAAZ Microparticles

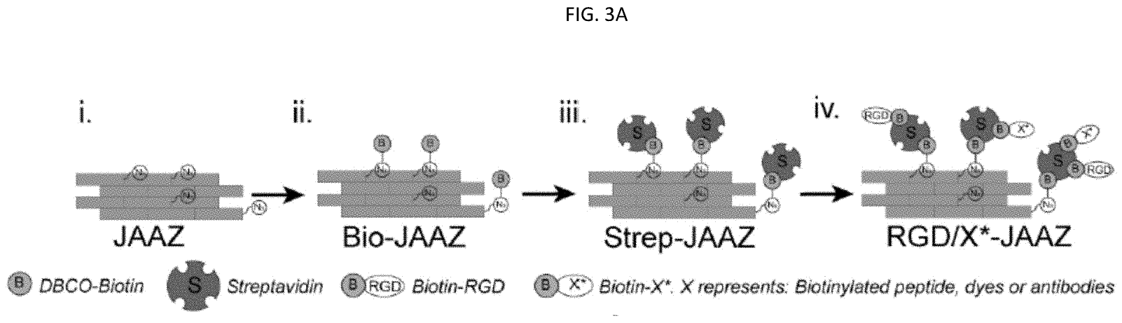

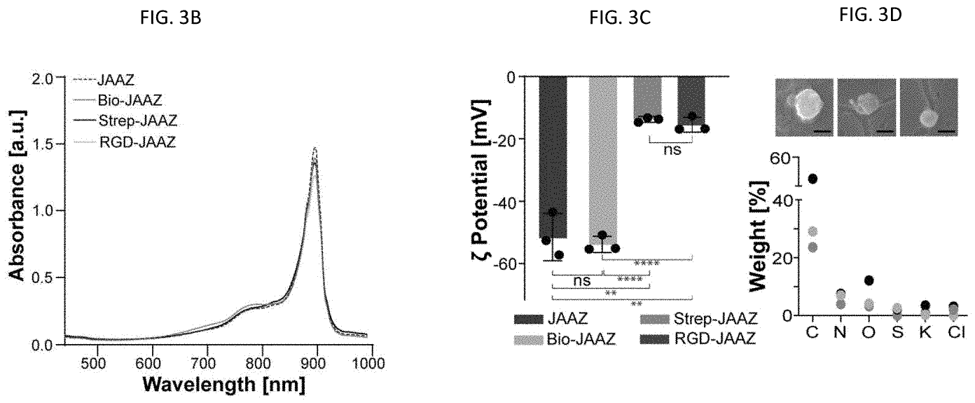

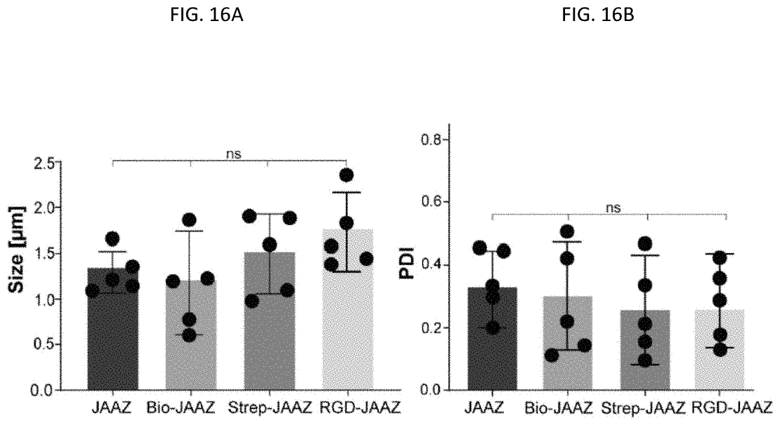

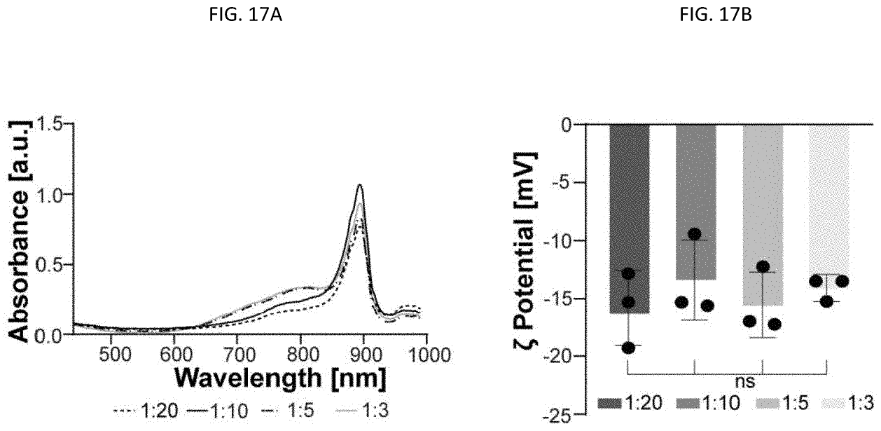

To enable conjugation of JAAZ with streptavidin, copper-free click chemistry was performed to graft biotin linkers onto the azide groups on the surface of the JAAZ particles. The biotin-streptavidin complex has been abundantly used in biotechnological applications due to its biocompatibility, strong binding affinity and availability of different biotinylated functional modalities for targeting or imaging. Copper free click chemistry was performed with the azide groups of JAAZ using a DBCO-PEG-biotin linker to create a biotinylated version of the JAAZ particles (Bio-JAAZ, FIG. 3A). An excess of DBCO-PEG-Biotin linker and then streptavidin was used to ensure complete modification of the surface (FIG. 3A). UV-Vis-NIR spectra of Bio-JAAZ and Strep-JAAZ show the typical J-aggregate absorption peak at 895 nm (FIG. 3B). After removing excess reactants using spin filtration, 77% of Bio-JAAZ particles and 65% of Strep-JAAZ particles were recovered (FIG. 15B). Upon addition of biotin, Bio-JAAZ particles exhibited a ζ potential of −54.1 mV (FIG. 3C), a non-significant change when compared to JAAZ particles. The attachment of streptavidin changed the ζ potential of the particles to −15.8 mV, making them significantly less charged than JAAZ, as seen in FIG. 3C. This reduction in zeta potential magnitude confirmed the presence of streptavidin on the surface of JAAZ particles. DLS measurements showed the size of Bio-JAAZ and Strep-JAAZ particles were 1.26±0.34 μm and 1.51±0.35 μm. respectively. Attachment of streptavidin caused no statistically significant increase in the size of JAAZ particles (FIG. 16A) confirming the absence of aggregation of the Strep-JAAZ particles. Strep-JAAZ particles have a PDI of 0.26 when compared to JAAZ particles with a PDI of 0.37 (FIG. 16B). To quantify the availability of biotin group, streptavidin was attached to JAAZ with different ratios (1:10, 1:5 and 1:3 ICG-azide:ICG molar ratio) and the concentration of streptavidin attached was measured using a bicinchoninic acid (BCA) assay. JAAZ particles with a molar ratio of 1:10 bound 166.2 μg/ml of streptavidin per 50 μM of free dye. The amount of streptavidin attached to 1:5 and 1:3 JAAZ particles were 623.1 and 510.6 μg/ml per 50 μM of free dye, respectively. JAAZ particles with no biotin groups demonstrated some non-specific binding of streptavidin but with ˜70% less streptavidin present when compared to the 1:10 molar ratio Bio-JAAZ particles. The increased concentration of streptavidin did not affect the J-aggregate absorbance peak of the JAAZ particles (FIG. 17A) and the zeta potential of all the Strep-JAAZ samples were ˜−14 mV due to the functionalized streptavidin (FIG. 17B). These results indicate that a higher degree of biomolecule functionalization can be achieved by varying the molar ratio of ICG-azide to ICG.

Attachment of biotinylated targeting molecules such as RGD was also explored (FIG. 3A). Samples with attached RGD were labeled as RGD-JAAZ. A summary of the concentration of biomolecules used to functionalize JAAZ to create Bio-JAAZ, Strep-JAAZ and RGD-JAAZ is provided in Table 2. The attachment of RGD to Strep-JAAZ did not alter the characteristic J-aggregate peak at 895 nm (FIG. 3B). After removing excess RGD-Biotin by filtration, 45% of RGD-JAAZ particles were recovered (FIG. 15B). The average ζ potential of RGD-JAAZ samples was −15.8 mV (FIG. 3C), which is a non-significant change from the ζ potential of Strep-JAAZ. DLS measurements revealed the size of RGD-JAAZ particles as 1.72±0.36 (FIG. 16A) with a PDI of 0.26 (FIG. 16B). No fluorescence emission was recorded from RGD-JAAZ particles, indicating that addition of biomolecules had no effect on the fluorescence properties of the aggregates (FIG. 11B). The modification of JAAZ microparticles with a soluble protein like streptavidin increased the solubility of the microparticles preventing sedimentation. JAAZ and Strep-JAAZ microparticles were left to sediment at room temperature on a benchtop. As seen in FIGS. 18A-18B, no sedimentation was observed for up to 8 hours with Strep-JAAZ, whereas a visible pellet was formed for the JAAZ particles. At 24 hours, Strep-JAAZ particles settled down at the bottom of the centrifuge tube (FIG. 18B). However, upon slight mixing, they returned into solution, whereas a visible pellet was observed for JAAZ microparticles at 24 hours (FIG. 18A), which required vortex mixing to be properly dispersed. Therefore, the change in surface charge due to streptavidin functionalization, concentration of attached streptavidin, and increased solubility of the aggregates demonstrate our capability to conjugate biomolecules to the JAAZ microparticles surface.

### Example 4: Synthesis, Characterization, and Functionalization of Nanoscale JAAZ (N-JAAZ)

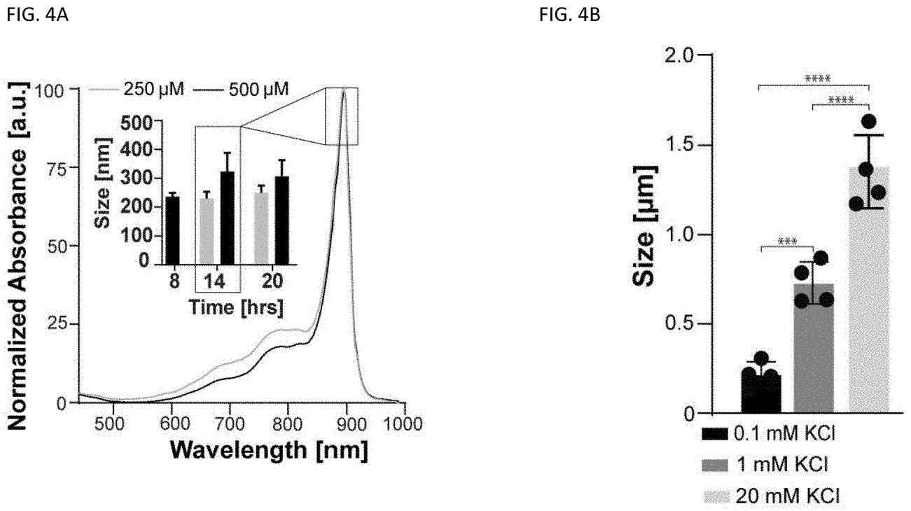

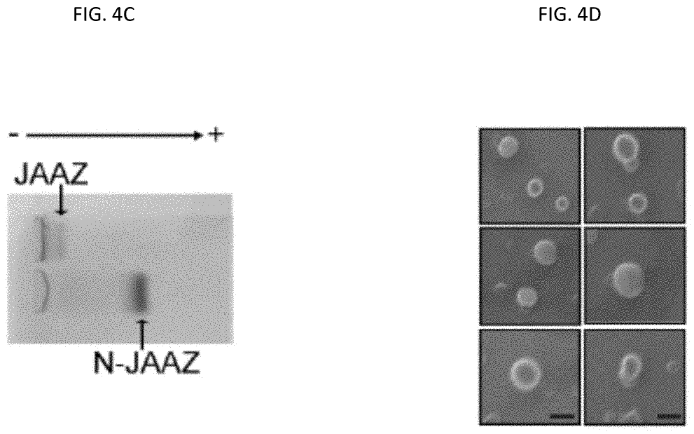

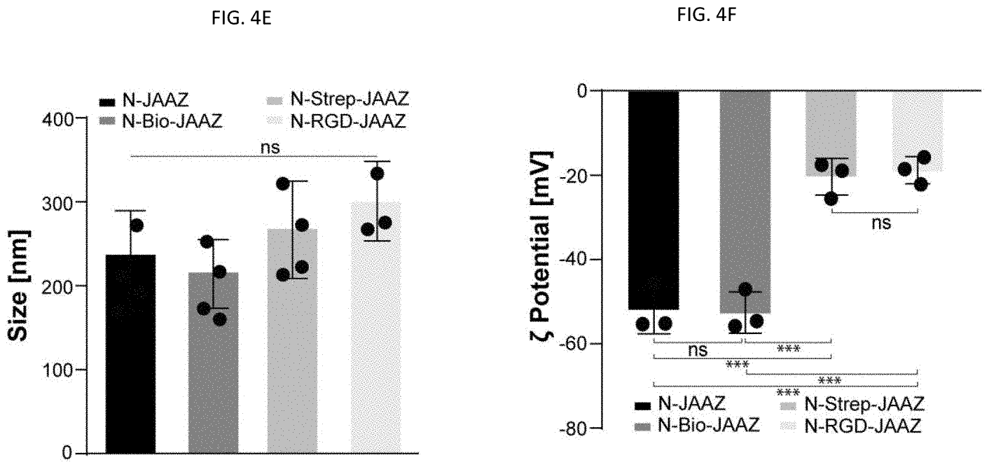

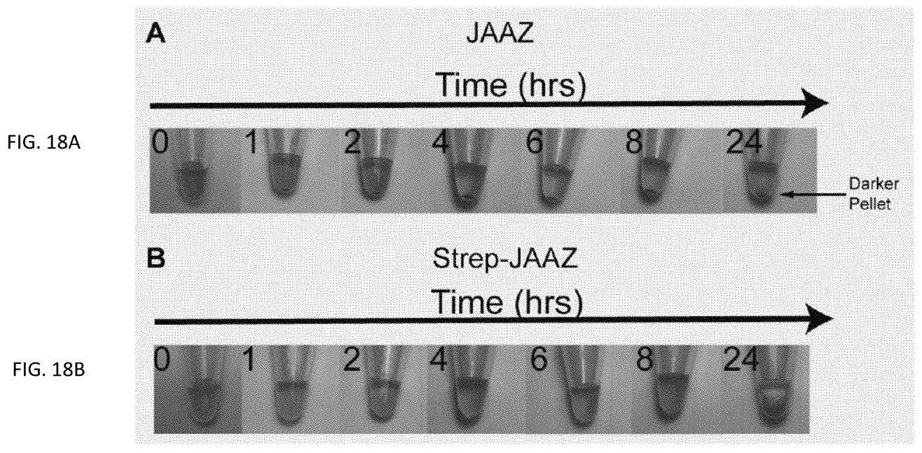

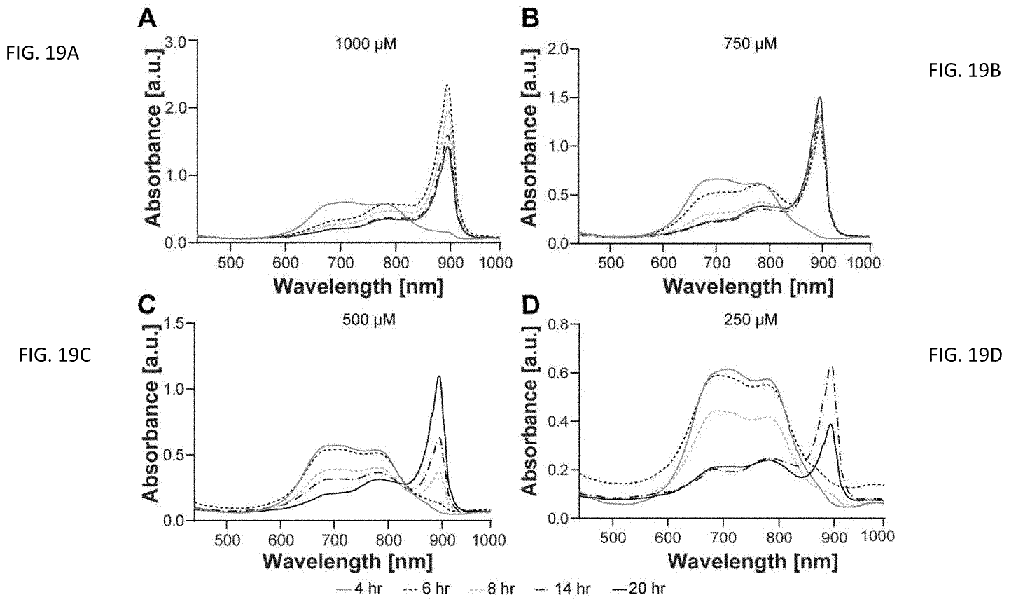

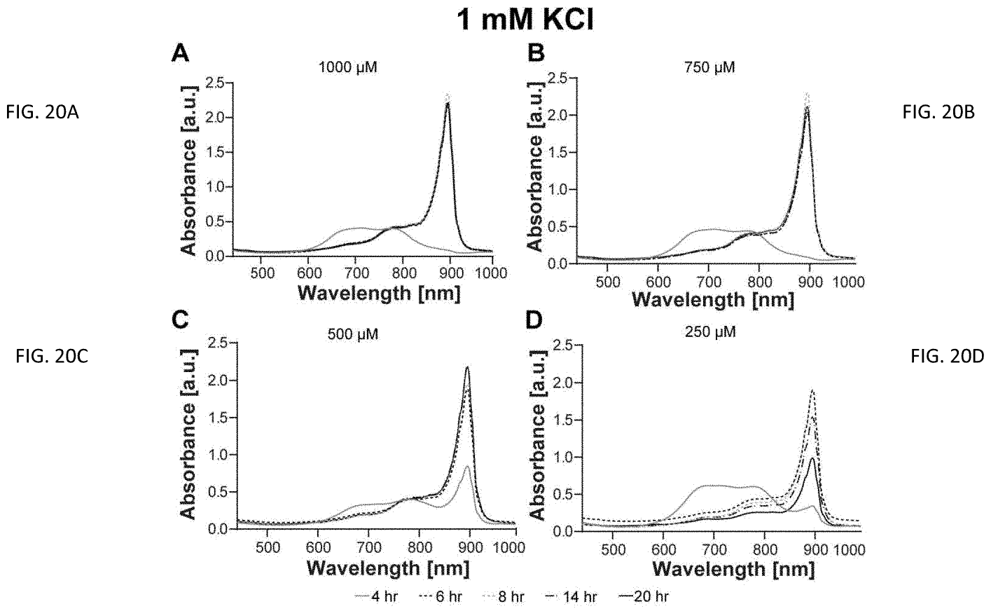

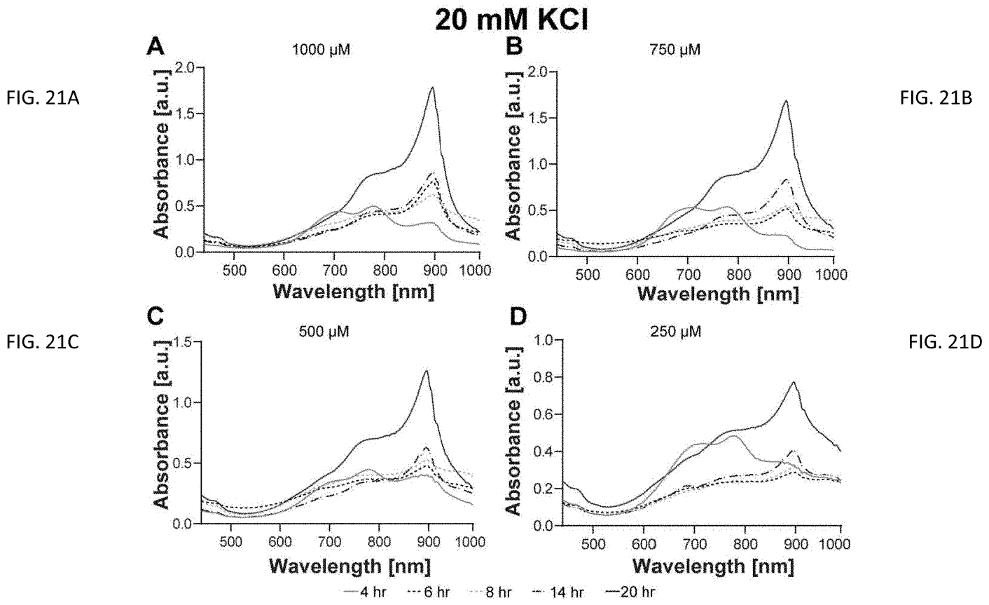

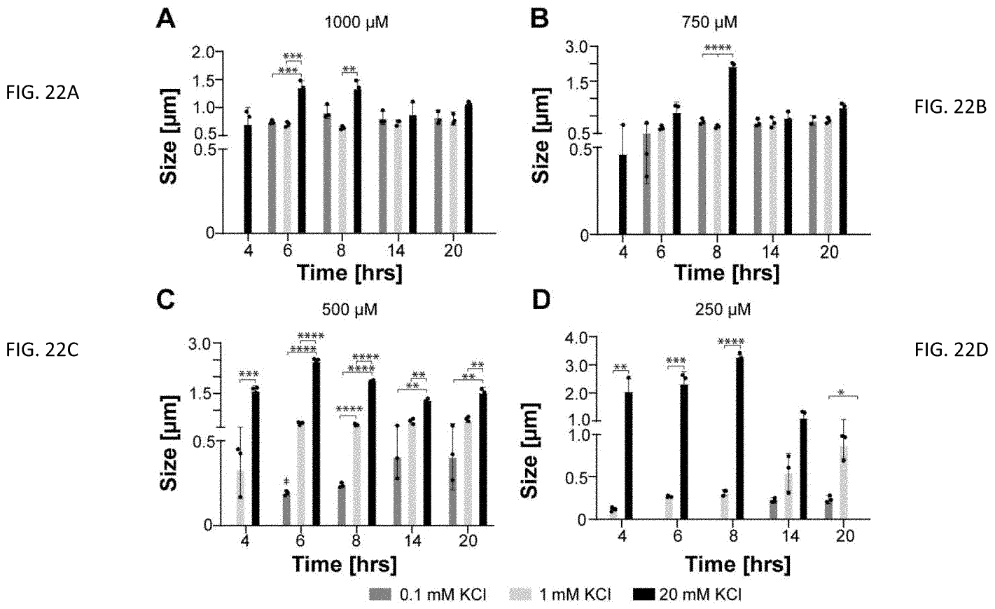

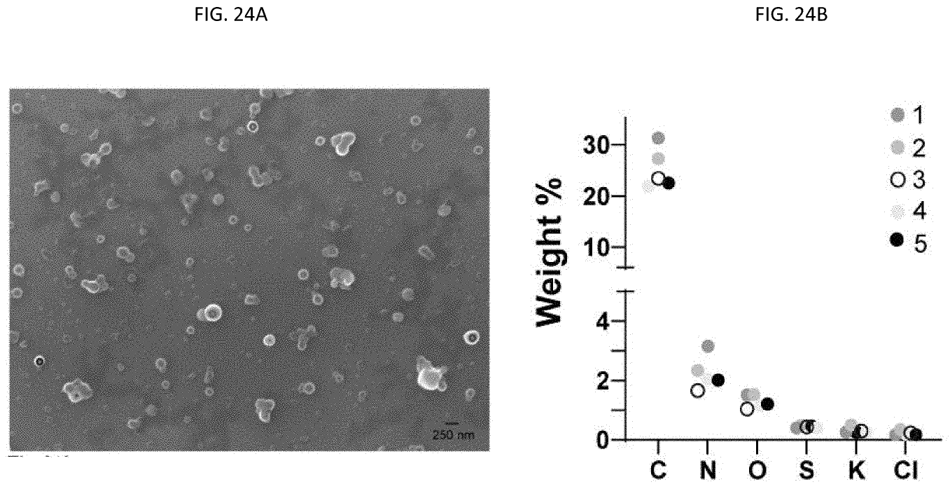

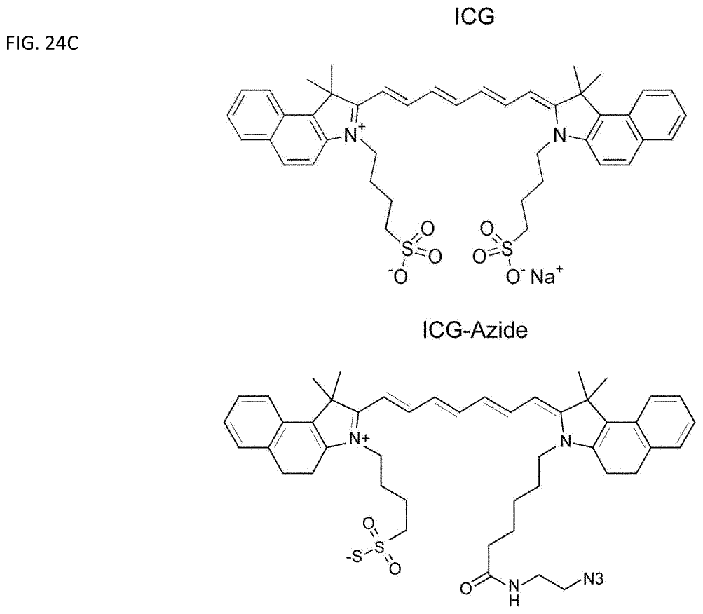

The micrometer scale of the JAAZ particles might restrict their use in applications that require the contrast agents to pass through biological barriers, for example, applications such as contrast-enhanced imaging of tumor lesions require nanosized particles. Therefore, to meet the requirements for such applications of J-aggregates, the synthesis protocol was adapted to enable control over the size of JAAZ particles. J-aggregate formation is dependent on time, salt and dye concentration; hence, the impact of these three parameters on the size of JAAZ particles was explored. Solutions of 250, 500, 750 and 1000 μM total dye with 1:10 ICG-azide:ICG molar ratio were incubated at 60° C. for 20 hours under KCl concentrations of 0.1, 1 and 20 mM. At 0.1 mM KCl, the 895 nm absorption peak appeared after incubation for 6 hours for 1000 and 750 μM of total dye (FIGS. 19A-19B). For 500 and 250 μM concentrations, 8 and 14 hours were required for the formation of J-aggregates, respectively (FIGS. 19C-19D). For 1 and 20 mM KCl, a J-aggregate peak formed after 6 hours for all dye concentrations (FIGS. 20 and 21). The size of the formed JAAZ particles was measured using DLS for the above-mentioned conditions after 20 hours. At higher total dye concentrations for all salt molarities, the size of the formed JAAZ particles ranged from 700 nm to 1.4 μm (FIGS. 22A-22B). On reducing the initial concentration of total dye, at lower salt molarities of 0.1 mM and 1 mM KCl, nanoscale-JAAZ (N-JAAZ) of average size ranging from 220 to 600 nm were formed (FIGS. 22C-22D). Using total dye concentrations of 500 and 250 μM in 0.1 mM KCl (FIG. 4A), the synthesis of 350 and 225 nm JAAZ nanoparticles, respectively, was demonstrated (FIG. 4A, inset). These results indicate that varying the concentration of total dye, concentration of KCl and incubation time can be used to tune the size of JAAZ particles. Hence, N-JAAZ particles were able to be synthesized with average size of 225 nm (FIG. 4B) and a PDI of 0.20 with 250 μM total dye and 0.1 mM KCl. Using a higher concentration of KCl and total dye (1 mM KCl, 750 μM dye), JAAZ particles of 640 nm were obtained (FIG. 4B). JAAZ microparticles of 1.3 μm, as shown in FIG. 2B and FIG. 4A, were formed in a solution of 20 mM KCl, 1000 μM total dye. A detailed summary of the size of JAAZ particles formed varying time, KCl and total dye concentrations is given in FIG. 23. Like JAAZ microparticles, N-JAAZ have no fluorescence emission (FIG. 11C). To demonstrate the difference in sizes between the JAAZ micro and nanoparticles, the two samples were loaded onto an agarose gel (FIG. 4C) where the negatively charged JAAZ and N-JAAZ particles should move toward the positive electrode. N-JAAZ particles of ˜220 nm moved through the gel and formed a distinct green band after traversing the gel as seen in FIG. 4C, whereas the size of JAAZ microparticles prevents them from migrating through the gel. These results confirm the size of N-JAAZ particles. The size of N-JAAZ particles was further confirmed by SEM images (FIG. 4D) which showed particles with an average diameter of 175 nm. EDX analysis (FIG. 24) confirmed the composition of N-JAAZ particles.

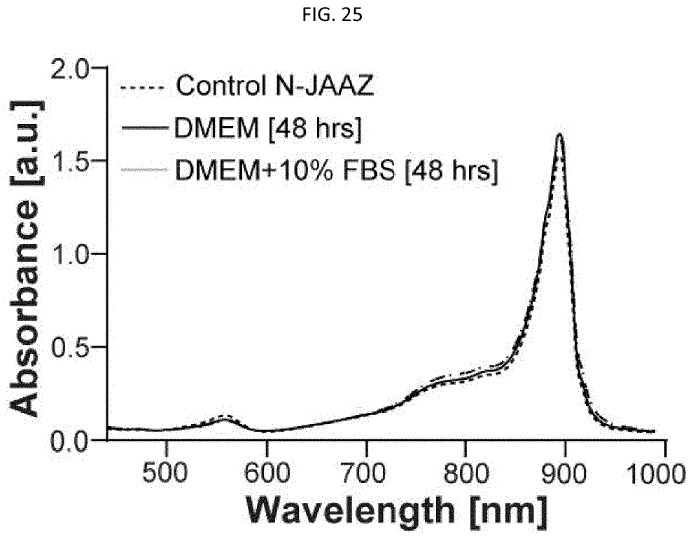

Following the same biomolecule functionalization strategy used for the microparticles (FIG. 3A), biotin and streptavidin were functionalized onto the surface of nanoscale JAAZ. The size of the JAAZ particles were measured after functionalization with streptavidin. After attaching DBCO-Biotin, the average hydrodynamic diameter of N-Bio-JAAZ particles were 210 nm; following streptavidin attachment N-Strep-JAAZ particles of 255 nm were obtained (FIG. 4E). After RGD attachment, the average size of N-RGD-JAAZ particles was 313 nm (FIG. 4E), a statistically non-significant increase compared to N-JAAZ particles. Hence, the synthesized N-JAAZ particles can be functionalized with biomolecules such as streptavidin and RGD yielding nanosized and targeted J-aggregates. As in the case of JAAZ microparticles, attachment of the slightly negatively charged streptavidin to N-JAAZ particles makes their zeta potential more positive. The average ζ potentials of-N-JAAZ, -N-Bio-JAAZ, N-Strep-JAAZ and N-RGD-JAAZ particles were −52 mV, −53 mV, −21 mV and −18 mV respectively (FIG. 4F). These values were comparable to those obtained for JAAZ microparticles. To determine if the reduction in size resulted in changes in the stability of the N-JAAZ particles, stability studies of these particles were performed in DMEM and DMEM+10% FBS. When incubated in both media for 48 hours at 37° C., no significant differences were observed in the absorbance spectra (FIG. 25). In addition, no significant change in absorbance, size or ζ potential were observed for N-JAAZ particles on vacuum drying and resuspension (FIG. 14A and Table 1), indicating that a reduced size has no notable influence on the stability of the aggregates. Thus, a facile way of controlling the size of JAAZ particles was demonstrated. Stable N-JAAZ can thus be modified with biomolecules using the same strategy applied to JAAZ microparticles, making targeted nanoscale J-aggregates which can be used for versatile imaging applications.

### Example 5: PA Properties of Different JAAZ Particles and Cellular Targeting Capabilities of RGD-JAAZ

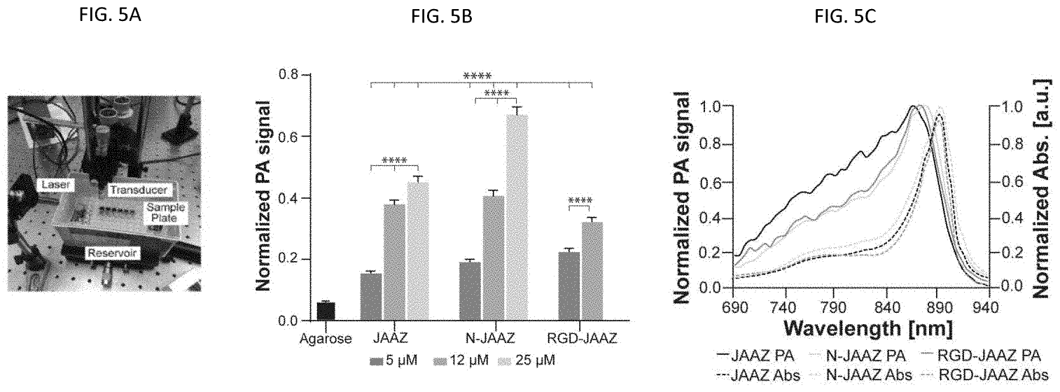

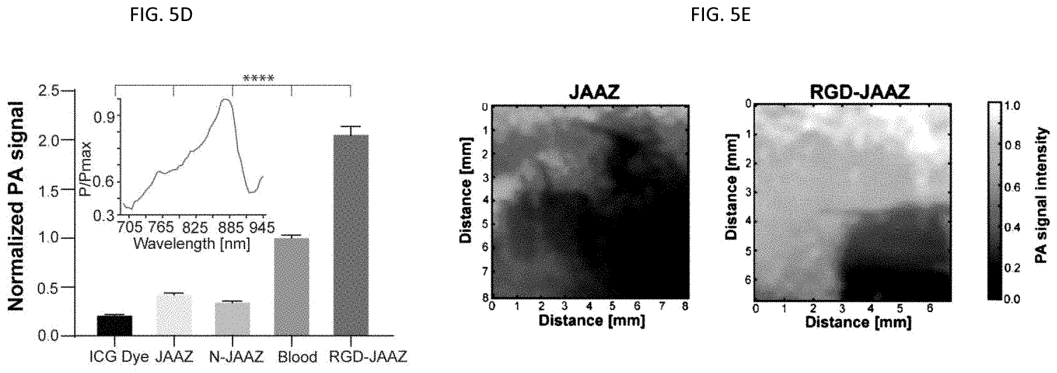

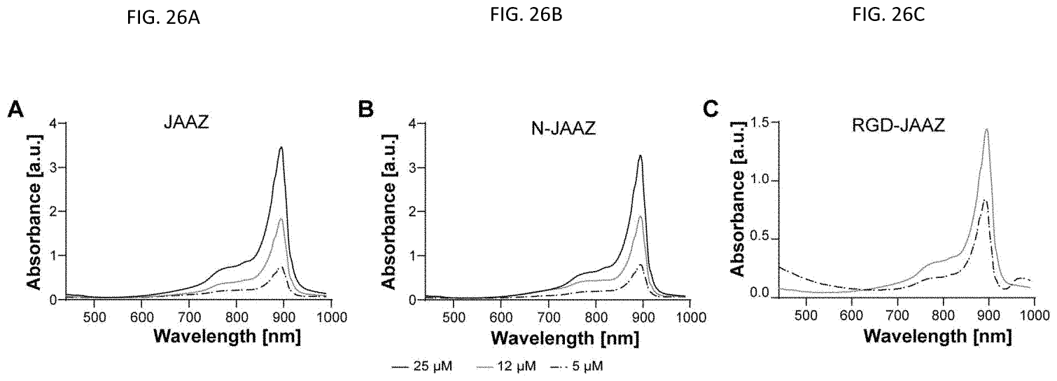

To quantify their viability for PAI applications, the in-vitro photoacoustic properties of micro and nanoscale JAAZ along with RGD functionalized JAAZ were characterized. FIG. 5A shows the photoacoustic setup used, which includes the different J-aggregate samples embedded in agarose and submerged in a water reservoir, illuminated by a pulsed laser. PA signal intensities were recorded for JAAZ and N-JAAZ particles at 25, 12 and 5 μM free dye concentration in 0.5% (w/v) agarose. For RGD-JAAZ samples, the PA signal was recorded at 12 and 5 μM free dye concentration. All PA signals were normalized with respect to 1:100 dilution of India Ink in water. The normalized PA signal intensity of N-JAAZ particles at the same dye concentration as that of JAAZ particles was slightly higher (FIG. 5B) suggesting that the size of the particles might influence the PA signal. The normalized PA signal of RGD-JAAZ microparticles was comparable to the PA signal of the non-functionalized particles, demonstrating that attaching a protein like streptavidin does not substantially affect the PA conversion efficiency of the J-aggregates (FIG. 5B). An increase in normalized PA signal intensity was observed with increasing total free dye concentration (5, 12 and 25 μM, FIG. 5B) used for assembling the nanoparticles, which established the correlation between the total free dye concentration and PA signal intensity. The individual absorbance curves of all samples (JAAZ, N-JAAZ and RGD-JAAZ) at the above-mentioned concentrations are provided in FIG. 26. The normalized absorbance of JAAZ and N-JAAZ showed a peak at 895 nm, characteristic of J-aggregates (FIG. 5C, dashed lines). The normalized PA profile matched the absorbance profile, with the PA signal peak at 890 nm (FIG. 5C, solid lines). The slight shift of the PA signal maxima from the absorbance maximum can be attributed to slight offset between the wavelength setting and output wavelength of the tunable pulsed laser. The photoacoustic spectrum of RGD-JAAZ has a PA signal maximum at 885 nm, which is in the NIR-I region (FIG. 5C), and optimally suited for deep-tissue imaging.

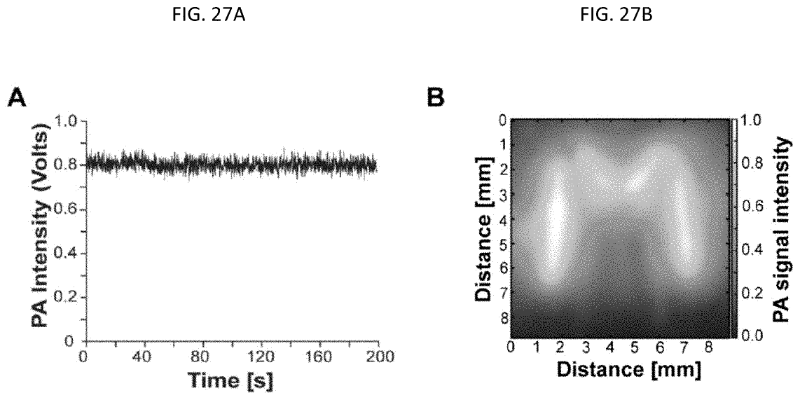

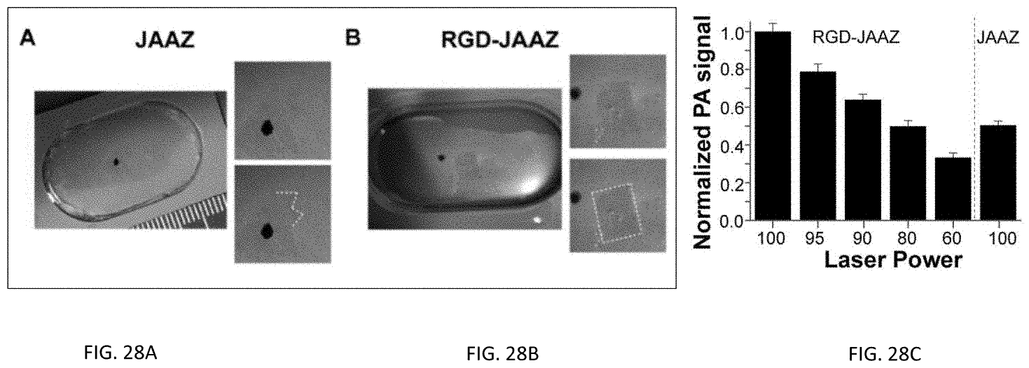

To explore the possibility of using JAAZ in vivo, the photoacoustic signal was compared to whole blood. Short segments of 24-gauge PTFE tubing (containing approximately100 μL, of sample) were filled with 20 μM of ICG dye, JAAZ, N-JAAZ, RGD-JAAZ, or whole sheep's blood. PA signals were recorded from each sample and the signal amplitude was normalized to the whole-blood PA-signal amplitude (FIG. 5D). The PA signal of 20 μM RGD-JAAZ was ˜2.1x that of blood excited at 895 nm, while 20 μM of free ICG dye was nearly ˜0.03x the intensity of whole blood at this wavelength. The PA-signal did not change in over 200 seconds when subjected to 2000 laser pulses (10 pulses/s) at 895 nm, which indicates that RGD-JAAZ are highly photostable (FIG. 27A). Short segments of PTFE tubing were filled with 10 μM RGD-JAAZ and arranged in an ‘M’ pattern. The ‘M’ pattern was scanned to create a 2D PA image (FIG. 27B). As a demonstration of cell labeling using JAAZ, HeLa cells were stained with 10 μM JAAZ and RGD-JAAZ particles on a custom glass slide with a red silicone insert. Before staining with JAAZ and RGD-JAAZ, an area of cells were scraped away using a razor blade as shown in FIG. 28. After washing the stained cells, a 35-MHz transducer was raster-scanned to produce a 2D PA image of the stained cells. The PA-color map indicates that the region where the cells were scraped away produced a weak PA signal that was less than 10% of the peak PA signal for cells stained with JAAZ and RGD-JAAZ (FIG. 5E). When comparing regions with similar density of seeded (100,000 cells/mL) cells on the slide, RGD-JAAZ labeling resulted in a more uniform labeling of the cells as well as significantly stronger PA signals (FIG. 5E) when compared to cells labeled with JAAZ (FIG. 5E). Additionally, the PA signal magnitude of the JAAZ stained cells decreases as the raster scan is completed (˜3 hours), whereas, compared with RGD-JAAZ stained cell culture, there is minimal to no diminution of the PA signal magnitude as the raster scan is completed (˜3 hours). The direct maximum PA signal from each stained cell culture was compared for different laser intensities for RGD-JAAZ vs JAAZ with the signal from JAAZ stained cells being only half that of cells stained with RGD-JAAZ (FIG. 28C).

### Example 6: Theranostic Applications of MJ Conjugates

An MJ can be further conjugated with a combination of a targeting moiety and a therapeutic cargo such as a chemotherapeutic drug (e.g., Doxorubicin, Daunorobicin), an antibody, a therapeutic protein, and a peptide. The cargo may be conjugated using a chemical linker such as that which was used for attachment of the targeting moiety.

These MJ conjugates are used as theranostic particles. They have targeted delivery capability to accumulate to any specific area of the body or specific diseased tissue using specific targeting moieties. They allow for imaging and diagnosis, and can be used to release drug on demand and with high spatial precision to treat diseased tissues. Ultrasound, light or a combination of both can be used to trigger dissolution of the MJ conjugate and the subsequent release of the drug in the body area of interest. The light can be pulsed or continuous.

Tables of P-Values for Indicated Figures

TABLE 3
P-values for FIG. 2A-2B

| | | |
|---|---|---|
| Row | JA | JAAZ |

FIG. 2A

| | | |
|---|---|---|
| JA | Na | 0.0001 |
| JAAZ | 0.0001 | Na |

FIG. 2B

| | | |
|---|---|---|
| JA | Na | 0.0161 |
| JAAZ | 0.0161 | Na |

TABLE 4
P-values for FIG. 3C
FIG. 3C

| Row | JAAZ | Bio-JAAZ | Strep-JAAZ | RGD-JAAZ |
|---|---|---|---|---|
| JAAZ | Na | 0.9058 | 0.0013 | 0.0019 |
| Bio-JAAZ | 0.9058 | Na | <0.0001 | <0.0001 |
| Strep-JAAZ | 0.0013 | <0.0001 | Na | 0.9011 |
| RGD-JAAZ | 0.0019 | <0.0001 | 0.9011 | Na |

TABLE 5
P-values for FIGS. 4B, 4E and 4F
FIG. 4B

| | | | |
|---|---|---|---|
| Row | 0.1 mM KCl | 1 mM KCl | 20 mM KCl |

| | | | | |
|---|---|---|---|---|
| 0.1 | mM KCl | Na | 0.0007 | <0.0001 |
| 1 | mM KCl | 0.0007 | Na | <0.0001 |
| 20 | mM KCl | <0.0001 | <0.0001 | Na |

| | | | | |
|---|---|---|---|---|
| Row | N-JAAZ | N-Bio-JAAZ | N-Strep-JAAZ | N-RGD-JAAZ |

FIG. 4E

| | | | | |
|---|---|---|---|---|
| N-JAAZ | Na | 0.3627 | 0.3634 | 0.0690 |
| N-Bio-JAAZ | 0.3627 | Na | 0.0645 | 0.0882 |
| N-Strep-JAAZ | 0.3634 | 0.0645 | Na | 0.0919 |
| N-RGD-JAAZ | 0.0690 | 0.0882 | 0.0919 | Na |

FIG. 4F

| | | | | |
|---|---|---|---|---|
| N-JAAZ | Na | 0.7620 | 0.0007 | 0.0004 |
| N-Bio-JAAZ | 0.7620 | Na | 0.0004 | 0.0002 |
| N-Strep-JAAZ | 0.0007 | 0.0004 | Na | 0.4006 |
| N-RGD-JAAZ | 0.0004 | 0.0002 | 0.4006 | Na |

TABLE 6
P-values for FIGS. 5B, 5D
FIG. 5B, 25 μM

| Row | JAAZ | N-JAAZ |
|---|---|---|
| JAAZ | Na | <0.0001 |
| N-JAAZ | <0.0001 | Na |

| | | | |
|---|---|---|---|
| Row | JAAZ | N-JAAZ | RGD-JAAZ |

FIG. 5B, 12 μM

| | | | |
|---|---|---|---|
| JAAZ | Na | <0.0001 | <0.0001 |
| N-JAAZ | <0.0001 | Na | <0.0001 |
| RGD-JAAZ | <0.0001 | <0.0001 | Na |

FIG. 5B, 5 μM

| | | | |
|---|---|---|---|
| JAAZ | Na | <0.0001 | <0.0001 |
| N-JAAZ | <0.0001 | Na | <0.0001 |
| RGD-JAAZ | <0.0001 | <0.0001 | <0.0001 |

FIG. 5D, 25 μM

| Row | ICG | JAAZ | N-JAAZ | RGD-JAAZ |
|---|---|---|---|---|
| ICG | Na | <0.0001 | <0.0001 | <0.0001 |
| JAAZ | <0.0001 | Na | <0.0001 | <0.0001 |
| N-JAAZ | <0.0001 | <0.0001 | Na | <0.0001 |
| RGD-JAAZ | <0.0001 | <0.0001 | <0.0001 | Na |

TABLE 7
P-values for FIG. 15B
FIG. 15B

| Row | Bio-JAAZ | Strep-JAAZ | RGD-JAAZ |
|---|---|---|---|
| Bio-JAAZ | Na | 0.4386 | 0.0358 |
| Strep-JAAZ | 0.4386 | Na | 0.0477 |
| RGD-JAAZ | 0.0358 | 0.0477 | Na |

TABLE 8
P-values for FIGS. 16A-16B

| | | | | |
|---|---|---|---|---|
| Row | JAAZ | Bio-JAAZ | Strep-JAAZ | RGD-JAAZ |

FIG. 16A

| | | | | |
|---|---|---|---|---|
| JAAZ | Na | 0.5494 | 0.8409 | 0.0940 |
| Bio-JAAZ | 0.5494 | Na | 0.5175 | 0.1036 |
| Strep-JAAZ | 0.8409 | 0.5175 | Na | 0.2148 |
| RGD-JAAZ | 0.0940 | 0.1036 | 0.2148 | Na |

FIG. 16B

| | | | | |
|---|---|---|---|---|
| JAAZ | Na | 0.9729 | 0.6851 | 0.3637 |
| Bio-JAAZ | 0.9729 | Na | 0.7482 | 0.4650 |
| Strep-JAAZ | 0.7482 | 0.7482 | Na | 0.7118 |
| RGD-JAAZ | 0.3637 | 0.4650 | 0.7118 | Na |

TABLE 9
P-values for FIG. 17B
FIG. 17B

| Row | 1:20 | 1:10 | 1:5 | 1:3 |
|---|---|---|---|---|
| 1:20 | Na | 0.2901 | 0.7246 | 0.2769 |
| 1:10 | 0.2901 | Na | 0.4064 | 0.7123 |
| 1:5 | 0.7246 | 0.4064 | Na | 0.4275 |
| 1:3 | 0.2749 | 0.7123 | 0.4275 | Na |

TABLE 10
P-values for FIGS. 22A-22D

| | | | |
|---|---|---|---|
| Row | 0.1 mM | 1 mM | 20 mM |

FIG. 22A, 1000 μM, 6 hrs

| | | | | |
|---|---|---|---|---|
| 0.1 | mM | Na | 0.2833 | 0.0007 |
| 1 | mM | 0.2833 | Na | 0.0006 |
| 20 | mM | 0.0007 | 0.0006 | Na |

FIG. 22A, 1000 μM, 8 hrs

| | | | | |
|---|---|---|---|---|
| 0.1 | mM | Na | 0.0157 | 0.0165 |
| 1 | mM | 0.0157 | Na | 0.0010 |
| 20 | mM | 0.0165 | 0.0010 | Na |

FIG. 22A, 1000 μM, 14 hrs

| | | | | |
|---|---|---|---|---|
| 0.1 | mM | Na | 0.2834 | 0.6409 |
| 1 | mM | 0.2834 | Na | 0.2570 |
| 20 | mM | 0.6409 | 0.2570 | Na |

FIG. 22A, 1000 μM, 20 hrs

| | | | | |
|---|---|---|---|---|
| 0.1 | mM | Na | 0.7945 | 0.0462 |
| 1 | mM | 0.7945 | Na | 0.0296 |
| 20 | mM | 0.0462 | 0.0296 | Na |

FIG. 22B, 750 μM, 6 hrs

| | | | | |
|---|---|---|---|---|
| 0.1 | mM | Na | 0.4137 | 0.0506 |
| 1 | mM | 0.4137 | Na | 0.0502 |
| 20 | mM | 0.0506 | 0.0502 | Na |

FIG. 22B, 750 μM, 8 hrs

| | | | | |
|---|---|---|---|---|
| 0.1 | mM | Na | 0.0428 | 0.00001 |
| 1 | mM | 0.0428 | Na | 0.0502 |
| 20 | mM | 0.00002 | 0.00001 | Na |

FIG. 22B, 750 μM, 14 hrs

| | | | | |
|---|---|---|---|---|
| 0.1 | mM | Na | 0.9776 | 0.2496 |
| 1 | mM | 0.0428 | Na | 0.3148 |
| 20 | mM | 0.2496 | 0.3148 | Na |

FIG. 22B, 750 μM, 20 hrs

| | | | | |
|---|---|---|---|---|
| 0.1 | mM | Na | 0.9776 | 0.0421 |
| 1 | mM | 0.9776 | Na | 0.0089 |
| 20 | mM | 0.0421 | 0.0089 | Na |

FIG. 22C, 500 μM, 4 hrs

| Row | 1 mM | 20 mM |
|---|---|---|
| 1 mM | Na | 0.0005 |
| 20 mM | 0.0005 | Na |

| | | | |
|---|---|---|---|
| Row | 0.1 mM | 1 mM | 20 mM |

FIG. 22C, 500 μM, 6 hrs

| | | | | |
|---|---|---|---|---|
| 0.1 | mM | Na | 0.00003 | 0.00007 |
| 1 | mM | 0.00003 | Na | 0.00002 |
| 20 | mM | 0.00007 | 0.00002 | Na |

FIG. 22C, 500 μM, 8 hrs

| | | | | |
|---|---|---|---|---|
| 0.1 | mM | Na | 0.00007 | <0.00001 |
| 1 | mM | 0.00007 | Na | 0.00002 |
| 20 | mM | <0.00001 | 0.00002 | Na |

FIG. 22C, 500 μM, 14 hrs

| | | | | |
|---|---|---|---|---|
| 0.1 | mM | Na | 0.0419 | 0.0030 |
| 1 | mM | 0.0419 | Na | 0.0022 |
| 20 | mM | 0.0030 | 0.0022 | Na |

FIG. 22C, 500 μM, 20 hrs

| | | | | |
|---|---|---|---|---|
| 0.1 | mM | Na | 0.0785 | 0.0055 |
| 1 | mM | 0.0785 | Na | 0.0013 |
| 20 | mM | 0.0055 | 0.0013 | Na |

| | | |
|---|---|---|
| Row | 1 mM | 20 mM |

FIG. 22D, 250 μM, 4 hrs

| | | | |
|---|---|---|---|
| 1 | mM | Na | 0.0013 |
| 20 | mM | 0.0013 | Na |

FIG. 22D, 250 μM, 6 hrs

| | | | |
|---|---|---|---|
| 1 | mM | Na | 0.0009 |
| 20 | mM | 0.0009 | Na |

FIG. 22D, 250 μM, 8 hrs

| | | | |
|---|---|---|---|
| 1 | mM | Na | 0.00003 |
| 20 | mM | 0.00003 | Na |

| | | | |
|---|---|---|---|
| Row | 0.1 mM | 1 mM | 20 mM |

FIG. 22D, 250 μM, 14 hrs

| | | | | |
|---|---|---|---|---|
| 0.1 | mM | Na | 0.1494 | 0.0322 |
| 1 | mM | 0.1494 | Na | 0.0627 |
| 20 | mM | 0.0322 | 0.0627 | Na |

FIG. 22D, 250 μM, 20 hrs

| | | | | |
|---|---|---|---|---|
| 0.1 | mM | Na | 0.0149 | 0.0186 |
| 1 | mM | 0.0149 | Na | 0.0530 |
| 20 | mM | 0.0186 | 0.0530 | Na |

### Example 7: In Vivo Imaging of Two Mice Using RGD Functionalized J-Aggregates

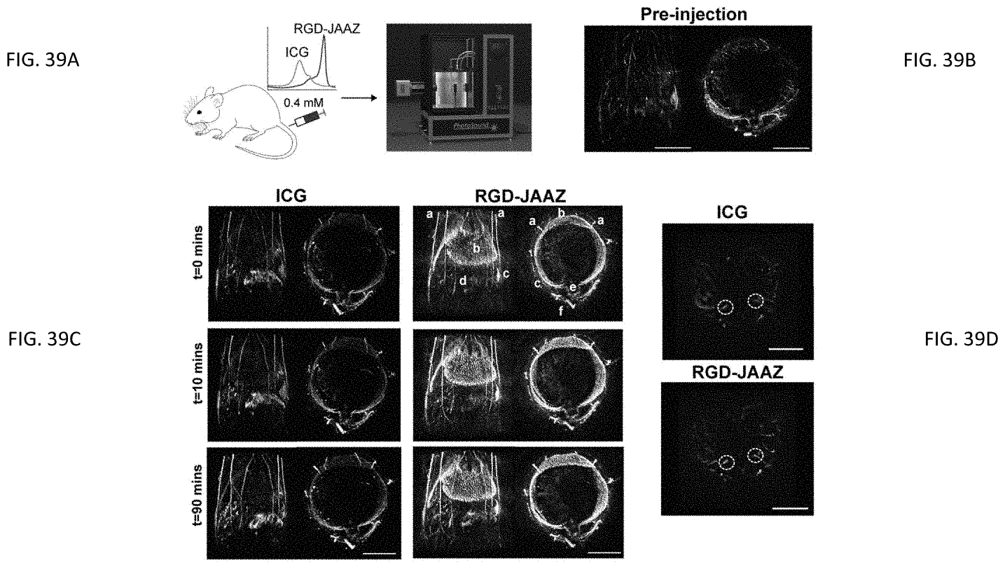

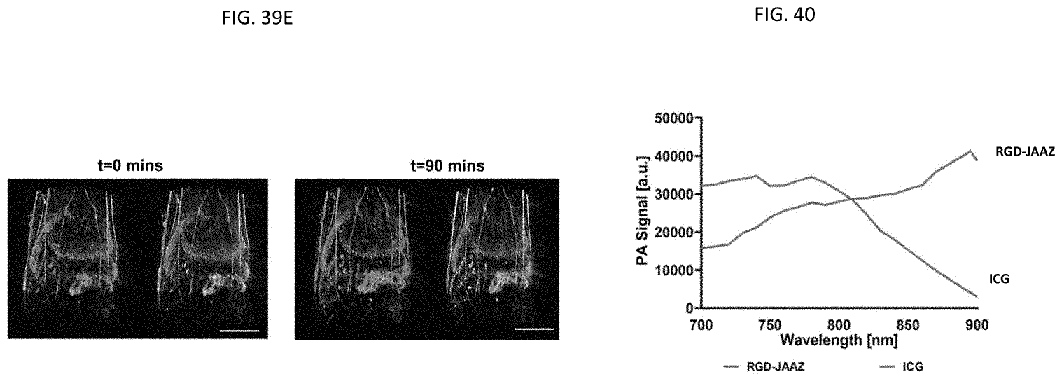

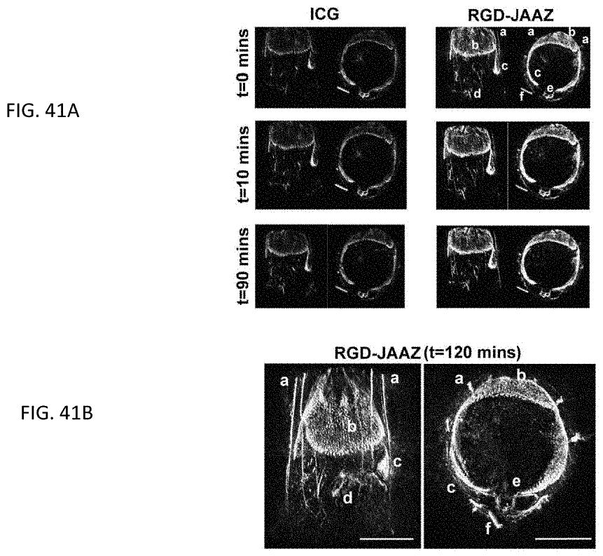

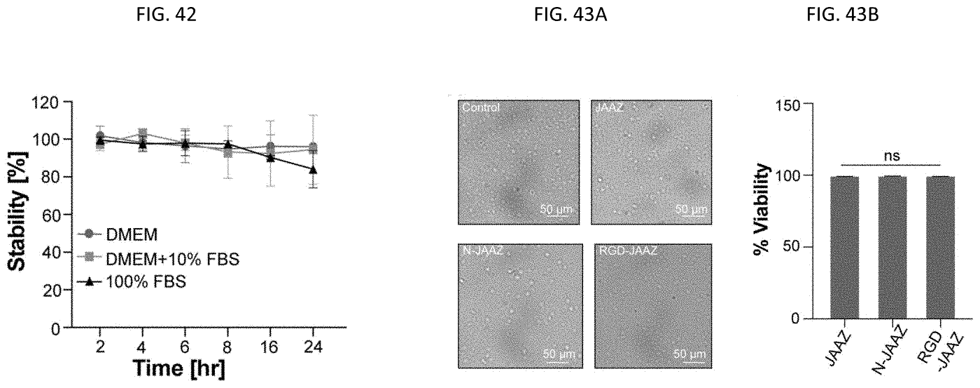

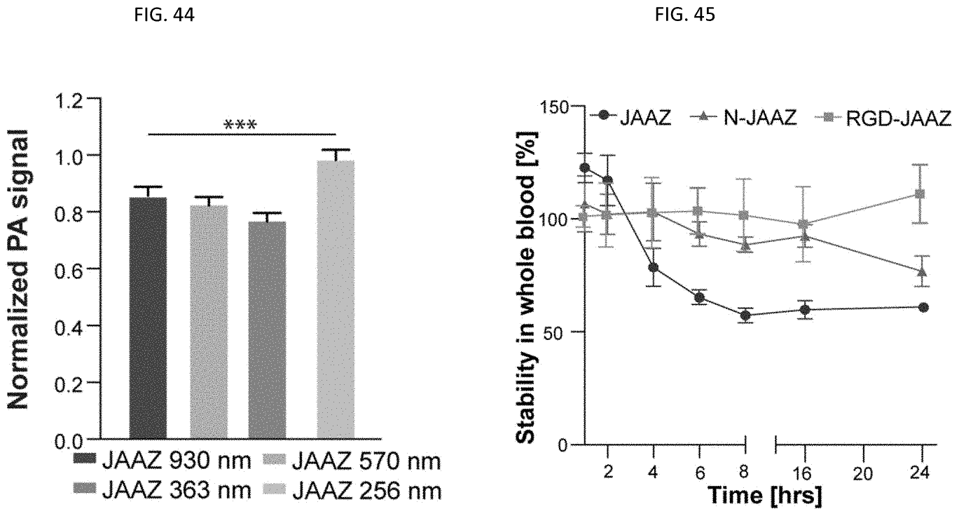

To further demonstrate the clinical translation potential of the JAAZ particles, an in vivo pilot study was conducted on the commercially available small animal PAI system TriTom from PhotoSound Technologies (Houston, Tex., USA) (FIG. 39A). We first acquired multiwavelength images of microcuvette tubes containing 0.4 mM of ICG dye or RGD-JAAZ particles to evaluate the PA spectra of both contrast agents. The peak PA signal in the RGD-JAAZ tube was measured at 895 nm while the ICG dye tube had peaks at 740 nm and 780 nm (FIG. 40). For in vivo imaging experiments, multispectral PAI were acquired in two female nu/nu nude mice at various timepoints (0, 10, 90 min) following tail-vein administration of ICG dye and RGD-JAAZ particles (FIGS. 39A-39D). A pre-injection baseline PA signal at 895 nm was also acquired for reference (FIG. 39B). We found that while RGD-JAAZ and ICG dye demonstrate a similar biodistribution in-vivo, intravenous administration of RGD-JAAZ particles improved visualization of blood-rich tissues such as the liver and spleen for up to 90-minutes post-injection (FIG. 39C and FIG. 41A for PA images of the second mouse) compared to ICG. The improved visualization with RGD-JAAZ particles continued for up to 120-minutes post-injection as seen in FIG. 41B. The achievable imaging depth was also improved by RGD-JAAZ particles demonstrated by the increased contrast-to-noise ratio (CNR) in blood vessels as deep as 5 mm from the surface of the skin compared to ICG dye 90-minutes post-injection (FIG. 6D and Table 11). The circulation half-life of RGD-JAAZ particles was 10 times greater than ICG dye (Table 11). Additionally, the molecular maps of oxyhemoglobin, deoxyhemoglobin, and RGD-JAAZ particles show the biodistribution of the contrast agent is restricted to the vasculature and blood-rich tissues such as the liver and spleen (FIG. 6E). Molecular maps were generated from a linear spectral unmixing of the multiwavelength PA images using previously reported spectra for hemoglobin and the measured spectra of the RGD-JAAZ particles at their injection concentration.

TABLE 11
Contrast-to-noise ratio and circulation
half-life of ICG and RGD-JAAZ particles.

| Injection | Contrast-to-noise ratio | Circulation half-life (mins) |
|---|---|---|
| ICG | 1.59 | 1.19 ± 0.45 |
| RGD-JAAZ | 2.42 | 10.26 ± 1.24 |

n = 2. Data is represented as mean ± SD.

### Example 8: Further Synthesis, Characterization, and Functionalization of Nanoscale JAAZ (N-JAAZ)

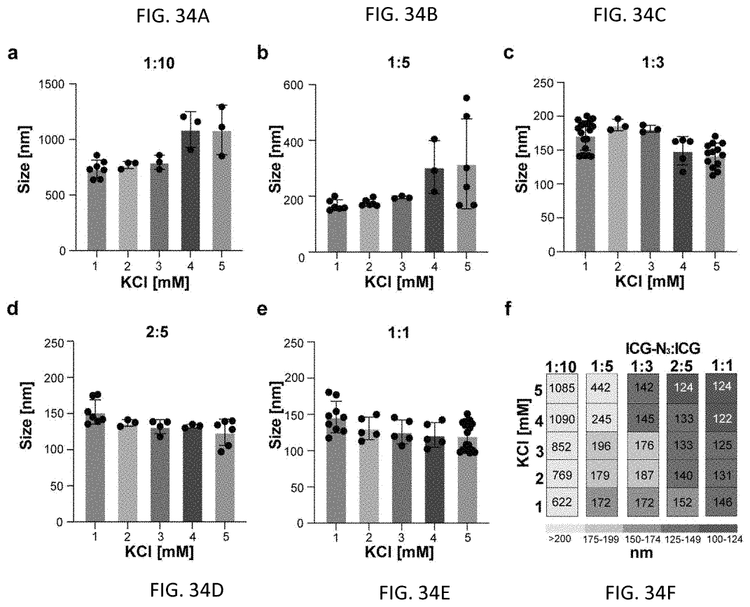

Stock solution of ICG dye was prepared at 5 mg/ml (6.5 mM) in water and stock of ICG-azide dye was prepared at 10 mg/ml (12 mM) in DMSO. Different molar ratio N-JAAZ particles, namely, 1:10, 1:5, 1:3, 2:5 and 1:1 was prepared by mixing the corresponding ratios of ICG and ICG-azide dye in different concentrations of potassium chloride. The final concentration of total dye (ICG and ICG-azide) was 350 μM for every sample. Unless stated otherwise, all samples were incubated for 18 hours at 60° C. In order to synthesize N-JAAZ particles of size ˜100 nm, a 350 μM total dye reaction solution of 1:1 ICG-azide:ICG molar ratio was incubated with 5 mM potassium chloride at the same temperature and time as specified previously. A summary of the different ratios of ICG-azide:ICG molar (1:10,1:5,1:3,2:5,1:1) and the different concentrations of potassium chloride (1,2,3,4.5 mM) used is given in FIG. 34F.

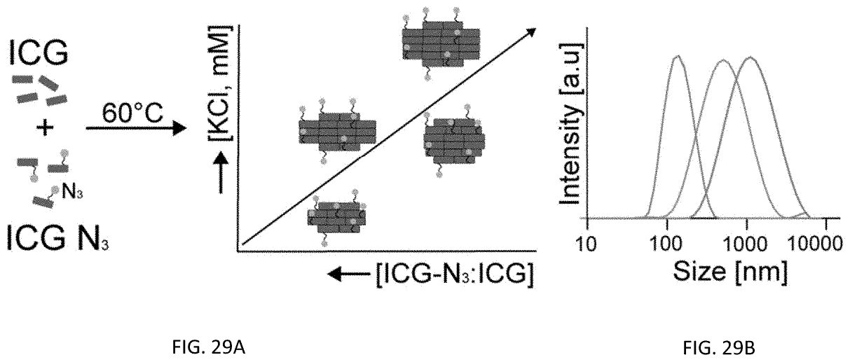

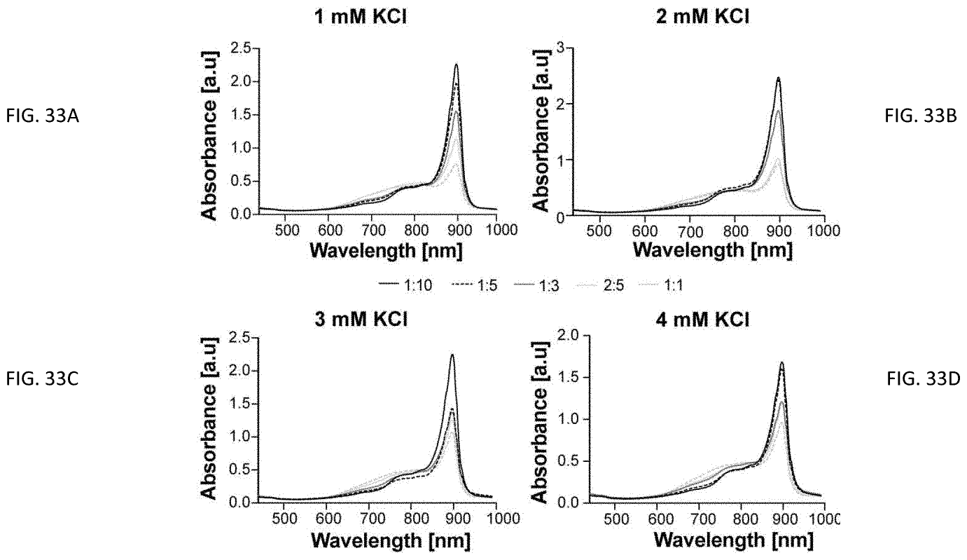

For the formation of N-JAAZ particles, different molar ratio solutions of ICG dye and azide modified ICG dye (ICG-azide) were incubated with different KCl concentrations at 60° C. (FIGS. 29A-29B). The conditions tested were 1:10, 1:5, 1:3, 2:5 and 1:1 of ICG-azide to ICG dye in 1 mM to 5 mM KCl concentrations. The final concentration of ICG-azide to ICG dye was 350 μM. Formation of J-aggregates for all above mentioned conditions was confirmed by the optical absorption peak at 895 nm in the UV-vis-NIR spectra. All molar ratios of ICG-azide:ICG tested formed J-aggregates after 18 hours incubation at 60° C. at 1, 2, 3, 4 mM KCl (FIGS. 33A-33D). Dynamic light scattering (DLS) was used for measurements of the size of all mentioned conditions. The hydrodynamic diameter of 1:10 molar ratio J-aggregates was 735±79.5 nm at 1 mM KCl and increased to 1089±160.4 nm at 4 mM KCl (FIG. 34A). At increased molar ratio of 1:5 ICG-azide:ICG, the hydrodynamic diameter of the formed J-aggregates decreased with sizes from 166±18.6 nm at 1 mM KCl to 304±94.9 nm (FIG. 34B). However, the formed nanosized J-aggregates at 1:5 molar ratio demonstrated a polydisperse population with a polydispersity index (PDI)>0.5 (Table 12).

TABLE 12
Polydispersity Index (PDI) of different molar ratio ICG-azid:ICG samples

| Molar ratio | Molarity of KCl [mM] | | | | |
|---|---|---|---|---|---|
| ICG-azide:ICG | 1 | 2 | 3 | 4 | 5 |
| 1:10 | 0.462 ± 0.066 | 0.423 ± 0.033 | 0.533 ± 0.077 | 0.533 ± 0.077 | 0.581 ± 0.095 |
| 1:5 | 0.413 ± 0.093 | 0.478 ± 0.098 | 0.727 ± 0.075 | 0.721 ± 0.082 | 0.691 ± 0.194 |
| 1:3 | 0.168 ± 0.022 | 0.163 ± 0.02 | 0.175 ± 0.016 | 0.202 ± 0.012 | 0.210 ± 0.024 |
| 2:5 | 0.124 ± 0.023 | 0.143 ± 0.023 | 0.151 ± 0.024 | 0.158 ± 0.013 | 0.143 ± 0.019 |
| 1:1 | 0.079 ± 0.011 | 0.087 ± 0.026 | 0.116 ± 0.019 | 0.127 ± 0.013 | 0.109 ± 0.016 |

n = 3 distinct samples; data is represented as mean ± SD.

On increasing the molar ratio of ICG-azide:ICG to 1:3 the size of the J-aggregates decreased to 171±21.7 nm, 187±8.5 nm, 181±5.3 nm and 149±20.8 nm at 1, 2, 3 and 4 mM KCl respectively (FIG. 34C). The PDI of the formed J-aggregates decreased to the range of ˜0.18±0.02. Hence, an increase in the molar ratio of ICG-azide:ICG formed a small sized, monodispersed aggregate population. We further increased the molar ratio of ICG-azide:ICG to 2:5 and 1:1 and incubated the solutions for 18 hours at 60° C. The size of the 2:5 molar ratio aggregates reduced from 152±16.7 nm at 1 mM KCl to 132±2.5 nm at 4 mM KCl, a —12% size reduction when compared to the 1:3 molar ratio samples (FIG. 34D). On further increasing the molar ratio to 1:1, the size of the aggregates was reduced from 146±21.6 nm to 121±16.9 nm (FIG. 34E). Therefore, an increase in the molar ratio of ICG-azide:ICG and the concentration of salt caused the formation of smaller J-aggregates. These nanosized J-aggregates retained their signature maximum absorption at 895 nm.

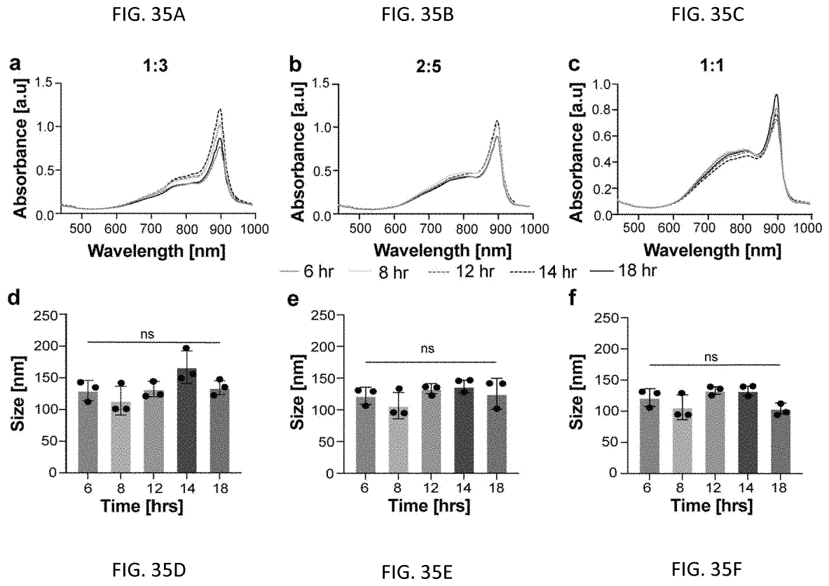

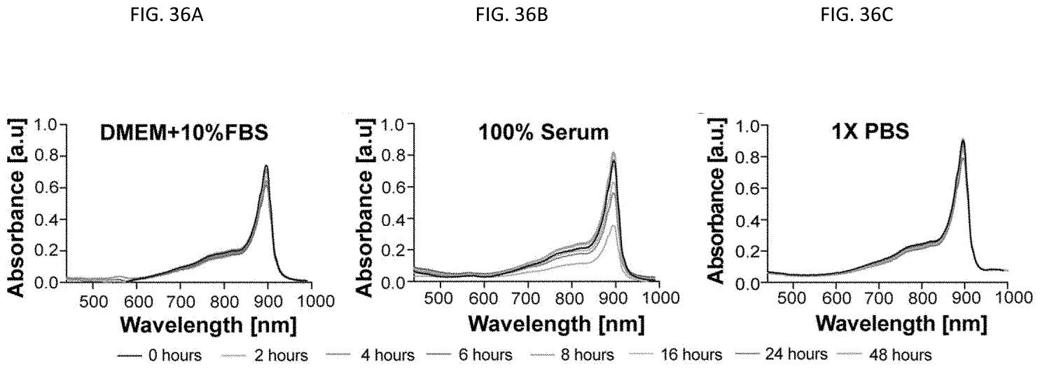

Along with molar ratio of the ICG and ICG-azide dye and concentration of KCl, the influence of the time of incubation was also studied for N-JAAZ formation. The absorbance spectra of 1:3, 2:5 and 1:1 molar ratio solution demonstrate the appearance of the J-aggregate peak at 895 nm at 6 hours (FIGS. 35A-35C). The intensity of the 895 nm absorbance peak remains like the peak intensity at 18 hours, signifying that 6 hour is enough for the formation of the J-aggregates. The size of the N-JAAZ particles formed at 6 hour is 130±15.9 nm, 122±13.6 nm, 125±8.4 nm for 1:3, 2:5 and 1:1 molar ratio, respectively (FIGS. 35D-35F). There is no increase in the size of the formed N-JAAZ particles till 18 hours which demonstrates that an incubation time of 6 hour at 60° C. is adequate for the formation of nanosized J-aggregates.

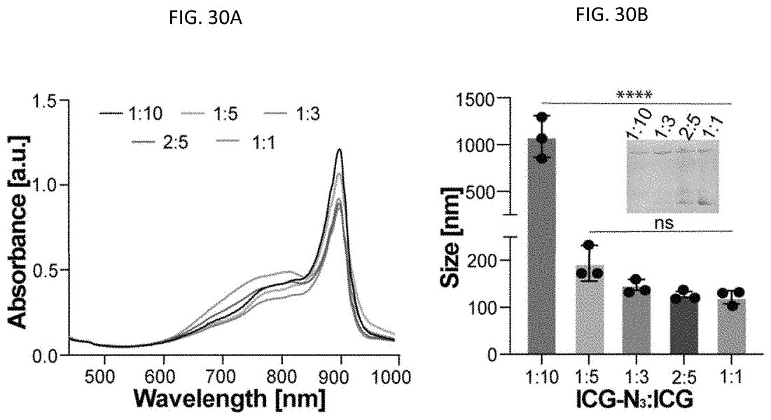

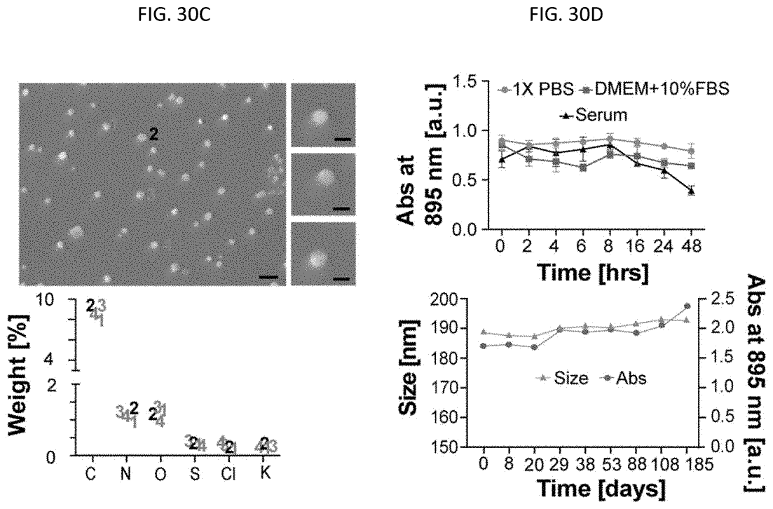

Hence, to demonstrate and summarize the effective control over the size of the nanosized J-aggregates (N-JAAZ), the above-mentioned ratios of ICG-azide:ICG (1:10, 1:5, 1:3, 2:5 and 1:1) were incubated at 60° C. in 5 mM KCl. All ratios demonstrated a maximum optical absorption at 895 nm (FIG. 30A) and the size of the N-JAAZ particles were 1085±222.5 nm, 189±37.5 nm, 147±11.6 nm, 127±6.3 nm and 121±13.8 for 1:10, 1:5, 1:3, 2:5 and 1:1 ratio respectively (FIG. 30B). The PDI of 1:10 and 1:5 molar ratio samples were >0.5, which indicates a polydisperse population distribution, whereas the PDI of 1:3, 2:5 and 1:1 ratio sample were below 0.2. Agarose gel electrophoresis (FIG. 30B, inset) demonstrates the difference between the sizes of 1:10, 1:3, 2:5 and 1:1 ratio N-JAAZ particles. A small population of polydisperse 1:10 molar ratio particles migrate from the well and form a faint band in the gel. 1:3 and 2:5 molar ratio particles migrate further in the gel and form a sharper band than 1:10 molar ratio particles. 1:1 molar ratio N-JAAZ particles form a dark green band in the gel representing a monodisperse population of nanoparticles. Scanning electron microcopy (SEM) images of N-JAAZ particles show a spherical particle morphology with an average size of 104±2.7 nm (FIG. 30C). Energy Dispersive Spectroscopy (EDS) analysis (FIG. 30C, bottom) showed the composition of the N-JAAZ particles as carbon, nitrogen, oxygen and sulfur which is the composition of ICG and ICG-azide dye.

The in-vitro stability of N-JAAZ particles was tested in physiological media such as 1X PBS at pH 7.4, media supplemented with 10% serum and 100% serum at 37° C. N-JAAZ particles were stable in 1×PBS and media with 10% serum for 48 hours with no decrease in the intensity of the absorbance peak at 895 nm (FIG. 30D). In 100% serum, a 50% decrease in the intensity of the absorbance peak at 895 nm was seen at 48 hours (FIG. 30D, black line). These results signify that the N-JAAZ particles are relatively stable under physiological conditions. Individual UV-Vis-NIR absorbance spectrums of N-JAAZ particles incubated in 1X PBS, media+10% FBS and 100% serum is provided in FIG. 35A-C. The absorbance at 895 nm and size of N-JAAZ particles was also recorded over 185 days to determine their long-term stability (FIG. 30D, bottom). There was no significant decrease in the peak intensity or change in the size of the J-aggregates. Hence, our synthesized N-JAAZ particles are stable when stored at 4° C. and protected from light for up to 6 months without any changes in their physical properties.

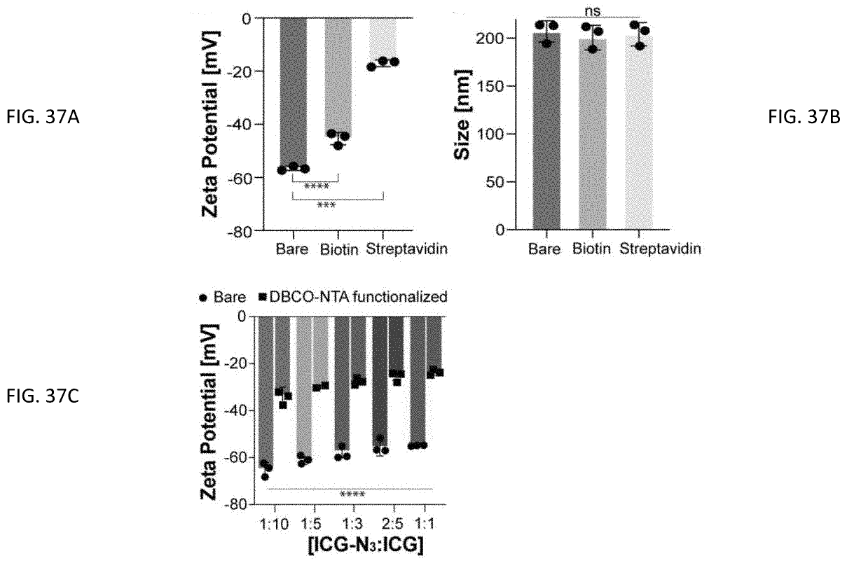

The incorporated azide groups in the synthesized N-JAAZ particles can be used for the attachment of targeting moieties by using click-chemistry groups. As described elsewhere herein, biomolecules such as biotin and streptavidin were functionalized onto the surface of micron sized JAAZ particles. Following the same protocol, DBCO-biotin followed by streptavidin was functionalized onto N-JAAZ particles. Zeta potential of the bare N-JAAZ particles was −56.6±4.2 mV. The addition of DBCO-biotin changed the zeta potential value to −45.3±5.4 mV. The zeta potential value changed to −17.4±2.4 mV on the coating of streptavidin onto the available biotin groups (FIG. 37A). The size of bare, biotin and streptavidin functionalized N-JAAZ particles were 207±10.3 nm, 200±11.1 nm and 204±10.7 nm respectively (FIG. 37B). There is no significant increase in size on the addition of streptavidin. Hence, these results demonstrate that the previously developed robust click-chemistry method for functionalization can be extended to the nanosized J-aggregates.

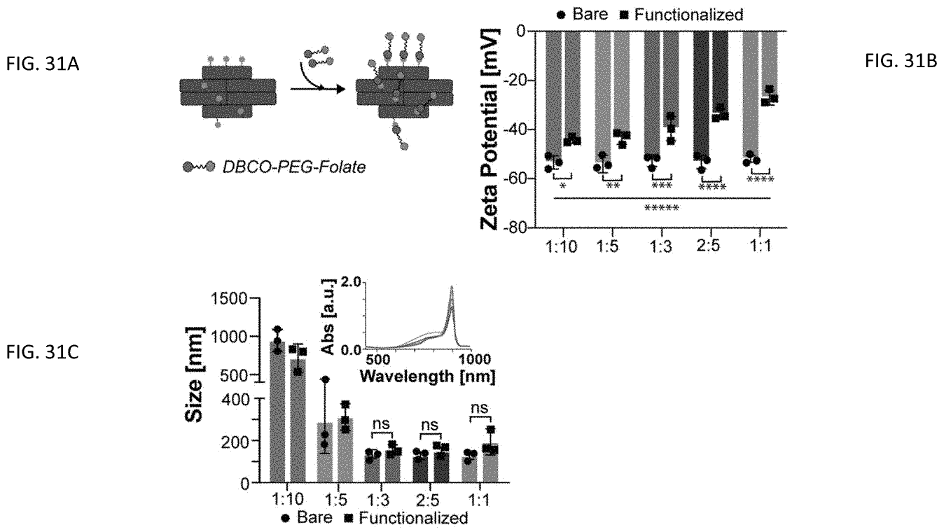

The versatility of N-JAAZ particles was further demonstrated by functionalizing the azide groups available with DBCO-nitriloaceticacid (DBCO-NTA). The NTA group interacts with histidine (His) and can be used for the attachment of His tagged proteins or peptides. Functionalization with more positively charged DBCO-NTA caused a significant change in the value of the zeta potential of 1:10, 1:5, 1:3, 2:5 and 1:1 molar ratio sample (FIG. 37C). N-JAAZ particles were then functionalized with folic acid, a folate receptor targeting ligand. Leveraging the surface azide groups, DBCO-PEG-Folate was added to the particles (FIG. 31A). The presence of the PEG group changed the surface charge of different ratio of N-JAAZ samples based on the molar ratio of ICG-azide modification. For 1:10, 1:5 and 1:3 ratio samples, the zeta potential of bare N-JAAZ particles was ˜53 mV, and upon functionalization, it changed to ˜−44 mV (FIG. 31B) which was a 10% increase. For 2:5 and 1:1 molar ratio particle, the zeta potential showed a ˜18% difference between the bare and DBCO-PEG-folate coated N-JAAZ particles due to greater number of surface azide groups present in 2:5 and 1:1 molar ratio particle. There was no significant increase in the size of the particles on functionalization with DBCO-PEG-Folate (FIG. 31C) for all molar ratios demonstrating functionalization does not change does not change the physical properties such as morphology of the N-JAAZ particles.

### Example 9: PA Properties of N-JAAZ Particles

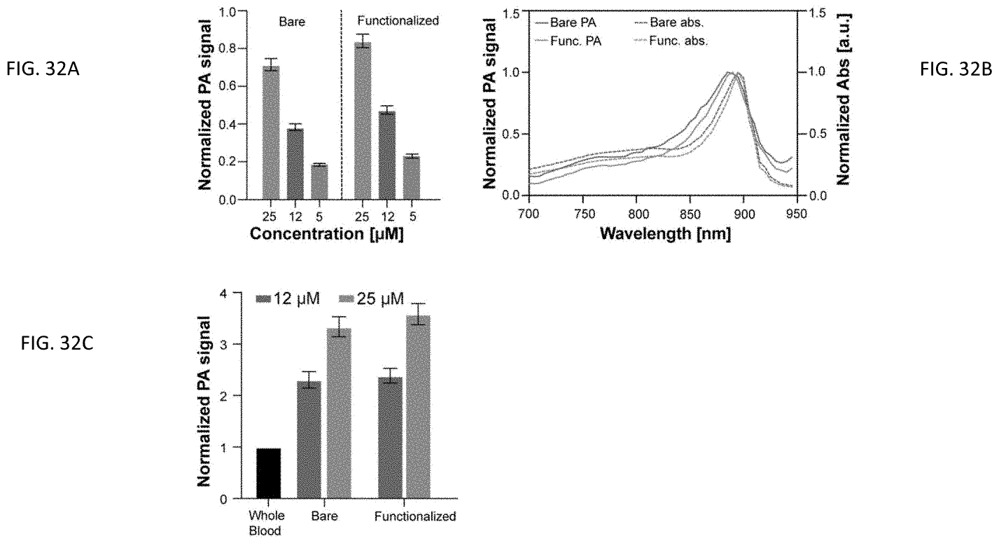

The in-vitro photoacoustic (PA) properties of the N-JAAZ particles were further evaluated. The PA signal magnitude of the particles showed a dependence on the equivalent concentration of free dye, as seen with absorbance. With increased free dye concentration (FIG. 32A), the PA signal had a proportional increase for bare and folate functionalized N-JAAZ particles. The N-JAAZ particles retained their 890 nm PA signal peak on the addition of folate molecules onto their surface (FIG. 32B), identical to the bare particles. Hence, this signifies that addition of folate receptors does not alter the PA properties of the N-JAAZ particles. For the further use of N-JAAZ particles as a contrast agent, it is important to record the PA signal of the particles when mixed with whole blood. Even at low concentrations (˜12 μM) of equivalent free dye, the PA signal magnitude of both bare and functionalized N-JAAZ particles was ˜2.5 times greater than the PA signal of whole blood (FIG. 32C). Therefore, the N-JAAZ particles can be used as a contrast agent since it can be detected against a common background chromophore which is hemoglobin.

### Example 10: Formation of J-Aggregates with Modified ICG Dyes

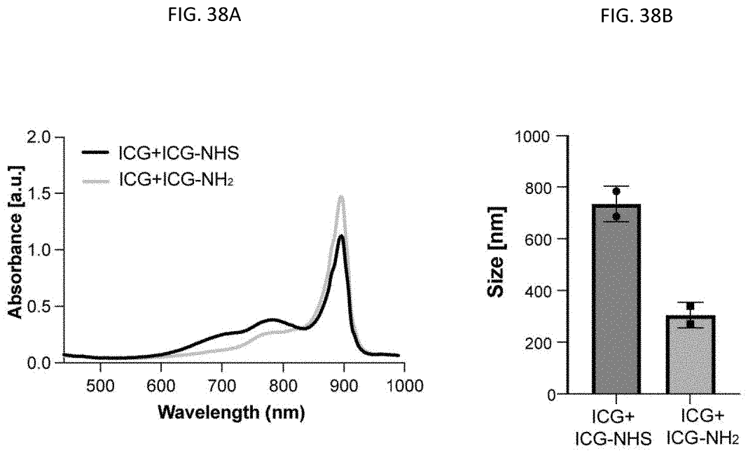

Other modified dyes with N-Hydroxysuccinimide (NHS) and amine (NH₂) functional groups were incorporated in a J-aggregate preparation to demonstrate that the method can work for dyes other than ICG-azide. Incorporating different functional groups also allowed the liberty to perform different functionalization chemistry onto the aggregates. The same method as described elsewhere herein (varying salt concentration) was used to prepare these aggregates. Results are shown in FIGS. 38A-38B. FIG. 38A shows UV-Vis-NIR spectrums demonstrating that the 895 nm J-aggregate peak is formed when ICG dye is incubated with ICG-NHS (black) or ICG-NH₂ (grey). FIG. 38B shows the hydrodynamic diameter measured using DLS of the J-aggregates formed by mixing ICG with commercially available ICG-NHS or ICG-NH₂ (AAT Bioquest, Sunnyvale, Calif., USA). The average diameter of ICG+ICG−NHS J-aggregates was 734±54 nm and that of ICG+ICG−NH₂ was 304±39 nm.

### Example 11: Tumor Margin Staining

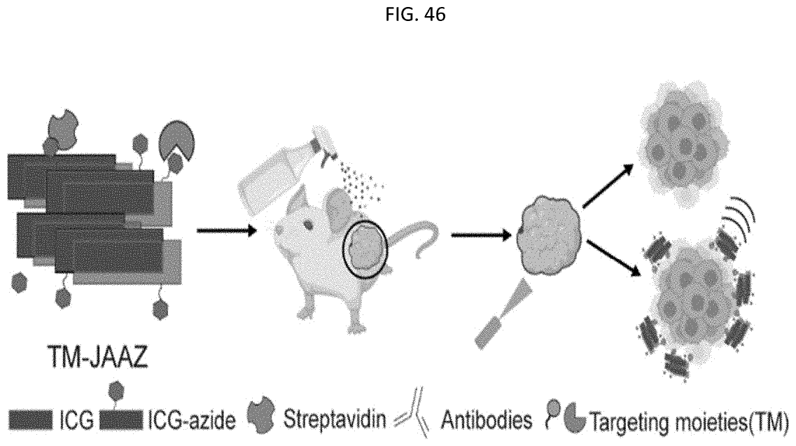

MJ or MJ conjugate is formulated in a water-based sprayable form (solvents can be water, PBS or other water-based physiological solution) that can be used for staining tumor margin directly after surgical resection of the tumor from the patient. The MJ or MJ conjugate may be micro or nanoscale. The MJ or MJ conjugate helps detect the presence of tumor cells near to the surface of the resected tumors via specific targeting moieties (adapted to the tumor targeted) that will enable tumor-cell specific labelling. The MJ or MJ conjugate is sprayed directly onto the tumor resects and the resects are washed with physiological solution after 5 to 10 minutes of staining. Only the tumor cells are labelled and non-tumor cells are not labelled. Using photoacoustic imaging, the surgeons and the pathologists are able to evaluate the presence of tumor cells close to the margin of the resected tumor and decide if there is a need to remove more tissues. (FIG. 46). This technique is applicable during surgery instead of after surgery, which will reduce the risk of secondary surgery.

### Example 12: Near-Infrared II (NIR-II) Imaging

NIR-II is the second NIR optical window that encompasses wavelengths ranging from 1000-1700 nm, respectively. Using these longer wavelengths to perform fluorescence imaging reduces the light scattering and autofluorescence typically observed in biological tissues and allows for deeper imaging with improved signal-to-background ratio. MJs and MJ conjugates have a strong and characteristic fluorescence emission signal detectable in the NIR-II windows when excited at wavelengths of 808 (that correspond to ICG absorption peak) and 890 nm (that correspond to ICG aggregates absorption peak).

### Results

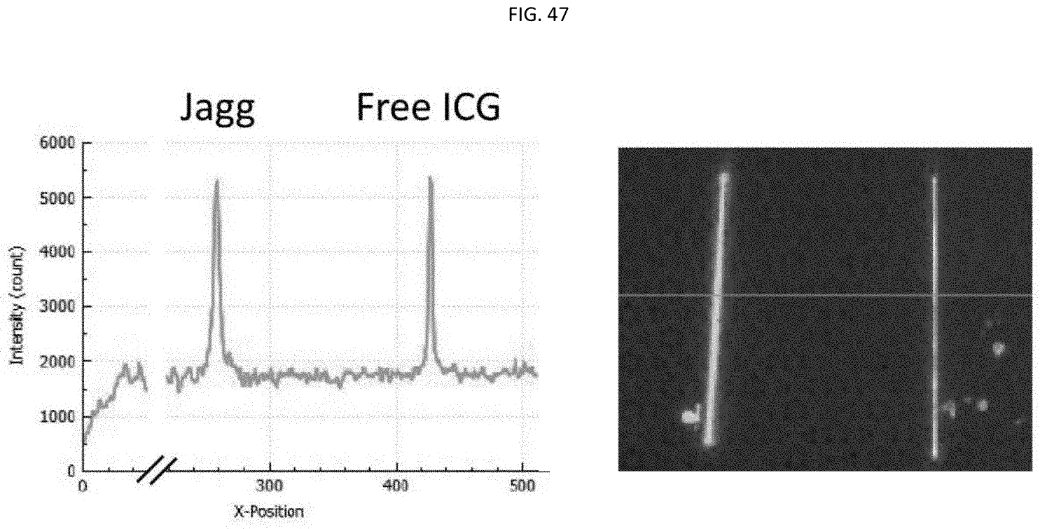

Free ICG (monomeric ICG) in PBS were compared with MJ in PBS at a same dye concentration (˜100 μM) placed in capillary tubes of 20 μL. A higher emission signal was shown from the MJs than from the free ICG in NIR-II (FIG. 47). Thus, MJs or MJ conjugates may be used to perform targeted deep in vivo imaging using dual imaging modality, namely photoacoustic and fluorescence imaging.

### BIBLIOGRAPHY

1. D. A. Sipkins, D. A. Cheresh, M. R. Kazemi, L. M. Nevin, M. D. Bednarski, K. C. P. Li, Detection of tumor angiogenesis in vivo by a v β 3 —targeted magnetic resonance imaging. Nat. Med. 4, 623-626 (1998).
2. K. Golman, R. in't Zandt, M. Lerche, R. Pehrson, J. H. Ardenkjaer-Larsen, Metabolic Imaging by Hyperpolarized 13C Magnetic Resonance Imaging for In vivo Tumor Diagnosis. Cancer Res. 66, 10855-10860 (2006).
3. T. Reuveni, M. Motiei, Z. Romman, A. Popovtzer, R. Popovtzer, Targeted gold nanoparticles enable molecular CT imaging of cancer: an in vivo study. Int. J. Nanomedicine. 6, 2859-2864 (2011).
4. C. Peng, L. Zheng, Q. Chen, M. Shen, R. Guo, H. Wang, X. Cao, G. Zhang, X. Shi, PEGylated dendrimer-entrapped gold nanoparticles for in vivo blood pool and tumor imaging by computed tomography. Biomaterials. 33, 1107-1119 (2012).
5. J. Zhang, C. Li, X. Zhang, S. Huo, S. Jin, F.-F. An, X. Wang, X. Xue, C. I. Okeke, G. Duan, F. Guo, X. Zhang, J. Hao, P. C. Wang, J. Zhang, X.-J. Liang, In vivo tumor-targeted dual-modal fluorescence/CT imaging using a nanoprobe co-loaded with an aggregation-induced emission dye and gold nanoparticles. Biomaterials. 42, 103-111 (2015).
6. S. K. Lyons, Advances in imaging mouse tumour models in vivo. J. Pathol. 205, 194-205 (2005).
7. T. Zhao, A. E. Desjardins, S. Ourselin, T. Vercauteren, W. Xia, Minimally invasive photoacoustic imaging: Current status and future perspectives. Photoacoustics. 16, 100146 (2019).
8. V. Ntziachristos, Going deeper than microscopy: the optical imaging frontier in biology. Nat. Methods. 7, 603-614 (2010).
9. P. Beard, Biomedical photoacoustic imaging. Interface Focus. 1, 602-631 (2011).
10. W. Shang, C. Zeng, Y. Du, H. Hui, X. Liang, C. Chi, K. Wang, Z. Wang, J. Tian, Core-Shell Gold Nanorod@Metal-Organic Framework Nanoprobes for Multimodality Diagnosis of Glioma. Adv. Mater. 29, 1604381 (2017).
11. L. Chen, J. Chen, S. Qiu, L. Wen, Y. Wu, Y. Hou, Y. Wang, J. Zeng, Y. Feng, Z. Li, H. Shan, M. Gao, Biodegradable Nanoagents with Short Biological Half-Life for SPECT/PAI/MRI Multimodality Imaging and PTT Therapy of Tumors. Small. 14, 1702700 (2018).
12. W. J. M. Mulder, D. W. J. van der Schaft, P. A. I. Hautvast, G. J. Strijkers, G. A. Koning, G. Storm, K. H. Mayo, A. W. Griffioen, K. Nicolay, Early in vivo assessment of angiostatic therapy efficacy by molecular MRI. FASEB J. 21, 378-383 (2007).
13. S. Mallidi, G. P. Luke, S. Emelianov, Photoacoustic imaging in cancer detection, diagnosis, and treatment guidance. Trends Biotechnol. 29, 213-221 (2011).
14. H. Prosch, A. Stadler, M. Schilling, S. Bürklin, E. Eisenhuber, E. Schober, G. Mostbeck, C.T. fluoroscopy-guided vs. multislice CT biopsy mode-guided lung biopsies: Accuracy, complications and radiation dose. Eur. J. Radiol. 81, 1029-1033 (2012).
15. M. Ishihara, M. S. M. d, A. H. M.d, H. S. M.d, H. T. M.d, K. Irisawa, T. Wada, T. A. M.d, in Photons Plus Ultrasound: Imaging and Sensing 2017 (International Society for Optics and Photonics, 2017; www.spiedigitallibrary.org/conference-proceedings-of-spie/10064/100642U/Possibility-of-620 transrectal-photoacoustic-imaging-guided-biopsy-for-detection-of/10.1117/12.2253277.short), vol. 621 10064, p. 100642U.
16. V. Koo, P. W. Hamilton, K. Williamson, Non-Invasive in vivo Imaging in Small Animal Research. Cell. Oncol. 28 (28), p. e245619.
17. A. Barandov, B. B. Bartelle, C. G. Williamson, E. S. Loucks, S. J. Lippard, A. Jasanoff, Sensing intracellular calcium ions using a manganese-based MRI contrast agent. Nat. Commun. 10, 897 (2019).
18. G. Huang, T. Zhao, C. Wang, K. Nham, Y. Xiong, X. Gao, Y. Wang, G. Hao, W.-P. Ge, X. Sun, B. D. Sumer, J. Gao, PET imaging of occult tumours by temporal integration of tumour-acidosis signals from pH-sensitive 64 Cu-labelled polymers. Nat. Biomed. Eng. 4, 314-324 (2020).
19. J. Weber, P. C. Beard, S. E. Bohndiek, Contrast agents for molecular photoacoustic imaging. Nat. Methods. 13, 639-650 (2016).
20. G. P. Luke, D. Yeager, S. Y. Emelianov, Biomedical Applications of Photoacoustic Imaging with Exogenous Contrast Agents. Ann. Biomed. Eng. 40, 422-437 (2012).
21. D. Wu, L. Huang, M. S. Jiang, H. Jiang, Contrast Agents for Photoacoustic and Thermoacoustic Imaging: A Review. Int. J. Mol. Sci. 15, 23616-23639 (2014).
22. G. Shan, R. Weissleder, S. A. Hilderbrand, Upconverting Organic Dye Doped Core-Shell Nano-Composites for Dual-Modality NIR Imaging and Photo-Thermal Therapy. Theranostics. 3, 267-274 (2013).
23. Z. Sheng, D. Hu, M. Zheng, P. Zhao, H. Liu, D. Gao, P. Gong, G. Gao, P. Zhang, Y. Ma, L. Cai, Smart Human Serum Albumin-Indocyanine Green Nanoparticles Generated by Programmed Assembly for Dual-Modal Imaging-Guided Cancer Synergistic Phototherapy. ACS Nano. 8, 12310-12322 (2014).
24. K. A. Homan, M. Souza, R. Truby, G. P. Luke, C. Green, E. Vreeland, S. Emelianov, Silver nanoplate contrast agents for in vivo molecular photoacoustic imaging. ACS Nano. 6,641-650 (2012).
25. J. Zhong, L. Wen, S. Yang, L. Xiang, Q. Chen, D. Xing, Imaging-guided high-efficient photoacoustic tumor therapy with targeting gold nanorods. Nanomedicine Nanotechnol. Biol. Med. 11,1499-1509 (2015).
26. K. Pu, J. Mei, J. V. Jokerst, G. Hong, A. L. Antaris, N. Chattopadhyay, A. J. Shuhendler, T. Kurosawa, Y. Zhou, S. S. Gambhir, Z. Bao, J. Rao, Diketopyrrolopyrrole-Based Semiconducting Polymer Nanoparticles for In Vivo Photoacoustic Imaging. Adv. Mater. 27,5184-5190 (2015).
27. Y. Zhou, Y.-S. Kim, D. E. Milenic, K. E. Baidoo, M. W. Brechbiel, In Vitro and In Vivo Analysis of Indocyanine Green-Labeled Panitumumab for Optical Imaging—A Cautionary Tale. Bioconjug. Chem. 25,1801-1810 (2014).
28. M. Capozza, F. Blasi, G. Valbusa, P. Oliva, C. Cabella, F. Buonsanti, A. Cordaro, L. Pizzuto, A. Maiocchi, L. Poggi, Photoacoustic imaging of integrin-overexpressing tumors using a novel ICG-based contrast agent in mice. Photoacoustics. 11,36-45 (2018).
29. V. Pansare, S. Hejazi, W. Faenza, R. K. Prud'homme, Review of Long-Wavelength Optical and NIR Imaging Materials: Contrast Agents, Fluorophores and Multifunctional Nano Carriers. Chem. Mater. Publ. Am. Chem. Soc. 24,812-827 (2012).
30. S. Luo, E. Zhang, Y. Su, T. Cheng, C. Shi, A review of NIR dyes in cancer targeting and imaging. Biomaterials. 32,7127-7138 (2011).
31. E. Najafzadeh, H. Ghadiri, M. Alimohamadi, P. Farnia, M. Mehrmohammadi, A. Ahmadian, Application of multi-wavelength technique for photoacoustic imaging to delineate tumor margins during maximum-safe resection of glioma: A preliminary simulation study. J. Clin. Neurosci. 70, 242-246 (2019).
32. B. Guo, Z. Feng, D. Hu, S. Xu, E. Middha, Y. Pan, C. Liu, H. Zheng, J. Qian, Z. Sheng, B. Liu, Precise Deciphering of Brain Vasculatures and Microscopic Tumors with Dual NIR-II Fluorescence and Photoacoustic Imaging. Adv. Mater. 31, 1902504 (2019).
33. Y. Zhu, L. A. Johnson, J. M. Rubin, H. Appelman, L. Ni, J. Yuan, X. Wang, P. D. R. Higgins, G. Xu, Strain-Photoacoustic Imaging as a Potential Tool for Characterizing Intestinal Fibrosis. Gastroenterology. 157, 1196-1198 (2019).
34. M. Heijblom, D. Piras, M. Brinkhuis, J. C. G. van Hespen, F. M. van den Engh, M. van der Schaaf, J. M. Klaase, T. G. van Leeuwen, W. Steenbergen, S. Manohar, Photoacoustic image patterns of breast carcinoma and comparisons with Magnetic Resonance Imaging and vascular stained histopathology. Sci. Rep. 5, 11778 (2015).
35. J. F. Lovell, C. S. Jin, E. Huynh, H. Jin, C. Kim, J. L. Rubinstein, W. C. W. Chan, W. Cao, L. V. Wang, G. Zheng, Porphysome nanovesicles generated by porphyrin bilayers for use as multimodal biophotonic contrast agents. Nat. Mater. 10, 324-332 (2011).
36. P. Huang, P. Rong, A. Jin, X. Yan, M. G. Zhang, J. Lin, H. Hu, Z. Wang, X. Yue, W. Li, G. Niu, W. Zeng, W. Wang, K. Zhou, X. Chen, Dye-loaded ferritin nanocages for multimodal imaging and photothermal therapy. Adv. Mater. Deerfield Beach Fla. 26, 6401-6408 (2014).
37. X. Tan, S. Luo, D. Wang, Y. Su, T. Cheng, C. Shi, A NIR heptamethine dye with intrinsic cancer targeting, imaging and photosensitizing properties. Biomaterials. 33, 2230-2239 (2012).
38. C.-K. Lim, J. Shin, Y.-D. Lee, J. Kim, K. S. Oh, S. H. Yuk, S. Y. Jeong, I. C. Kwon, S. Kim, Phthalocyanine-Aggregated Polymeric Nanoparticles as Tumor-Homing Near-Infrared Absorbers for Photothermal Therapy of Cancer. Theranostics. 2, 871-879 (2012).
39. S. Wang, J. Lin, T. Wang, X. Chen, P. Huang, Recent Advances in Photoacoustic Imaging for Deep-Tissue Biomedical Applications. Theranostics. 6, 2394-2413 (2016).
40. X. Song, R. Zhang, C. Liang, Q. Chen, H. Gong, Z. Liu, Nano-assemblies of J-aggregates based on a NIR dye as a multifunctional drug carrier for combination cancer therapy. Biomaterials. 57,84-92 (2015).
41. H.-W. An, S.-L. Qiao, C.-Y. Hou, Y.-X. Lin, L.-L. Li, H.-Y. Xie, Y. Wang, L. Wang, H. Wang, Self-assembled NIR nanovesicles for long-term photoacoustic imaging in vivo. Chem. Commun. 51,13488-13491 (2015).
42. E. E. Jelley, Molecular, Nematic and Crystal States of I: I-Diethyl—Cyanine Chloride. Nature. 139,631-631 (1937).
43. E. E. Jelley, Spectral Absorption and Fluorescence of Dyes in the Molecular State. Nature. 138,1009-1010 (1936).
44. F. Rotermund, R. Weigand, A. Penzkofer, J-aggregation and disaggregation of indocyanine green in water. Chem. Phys. 220,385-392 (1997).
45. R. Liu, J. Tang, Y. Xu, Y. Zhou, Z. Dai, Nano-sized Indocyanine Green J-aggregate as a One-component Theranostic Agent. Nanotheranostics. 1,430-439 (2017).
46. X. Song, H. Gong, T. Liu, L. Cheng, C. Wang, X. Sun, C. Liang, Z. Liu, J-Aggregates of Organic Dye Molecules Complexed with Iron Oxide Nanoparticles for Imaging-Guided Photothermal Therapy Under 915-nm Light. Small. 10,4362-4370 (2014).
47. B. Changalvaie, S. Han, E. Moaseri, F. Scaletti, L. Truong, R. Caplan, A. Cao, R. Bouchard, T. M. Truskett, K. V. Sokolov, K. P. Johnston, Indocyanine Green J Aggregates in Polymersomes for Near-Infrared Photoacoustic Imaging. ACS Appl. Mater. Interfaces. 11, 46437-46450 (2019).
48. C. C. L. Cheung, G. Ma, K. Karatasos, J. Seitsonen, J. Ruokolainen, C.-R. Koffi, H. A. F. M. Hassan, W. T. Al-Jamal, Liposome-Templated Indocyanine Green J-Aggregates for In Vivo Near-Infrared Imaging and Stable Photothermal Heating. Nanotheranostics. 4,91-106 (2020).
49. D. Miranda, H. Huang, H. Kang, Y. Zhan, D. Wang, Y. Zhou, J. Geng, H. I. Kilian, W. Stiles, A. Razi, J. Ortega, J. Xia, H. S. Choi, J. F. Lovell, Highly-Soluble Cyanine J-aggregates Entrapped by Liposomes for In Vivo Optical Imaging around 930 nm. Theranostics. 9, 381-390 (2019).
50. R. Zhang, Y. Xu, Y. Zhang, H. S. Kim, A. Sharma, J. Gao, G. Yang, J. S. Kim, Y. Sun, Rational design of a multifunctional molecular dye for dual-modal NIR-II/photoacoustic imaging and photothermal therapy. Chem. Sci. 10, 8348-8353 (2019).
51. L. Sun, J. Ding, W. Xing, Y. Gai, J. Sheng, D. Zeng, Novel Strategy for Preparing Dual-Modality Optical/PET Imaging Probes via Photo-Click Chemistry. Bioconjug. Chem. 27, 1200-1204 (2016).
52. Eugene. Lieber, C. N. R. Rao, T. S. Chao, C. W. W. Hoffman, Infrared Spectra of Organic Azides. Anal. Chem. 29, 916-918 (1957).
53. S. M. Mooi, S. N. Keller, B. Heyne, Forcing Aggregation of Cyanine Dyes with Salts: A Fine Line between Dimers and Higher Ordered Aggregates. Langmuir. 30, 9654-9662 (2014).
54. T. D. Slavnova, A. K. Chibisov, H. Görner, Kinetics of Salt-Induced J-aggregation of Cyanine Dyes. J. Phys. Chem. A. 109, 4758-4765 (2005).
55. I. Struganova, Dynamics of Formation of 1,1‘-Diethyl-2,2’-Cyanine Iodide J-Aggregates in Solution. J. Phys. Chem. A. 104, 9670-9674 (2000).
56. C. Shao, F. Xiao, H. Guo, J. Yu, D. Jin, C. Wu, L. Xi, L. Tian, Utilizing Polymer Micelle to Control Dye J-aggregation and Enhance Its Theranostic Capability. iScience. 22, 229-239 (2019).
57. S. M. Sedlak, L. C. Schendel, H. E. Gaub, R. C. Bernardi, Streptavidin/biotin: Tethering geometry defines unbinding mechanics. Sci. Adv. 6, eaay5999 (2020).
58. M. J. Mitchell, R. K. Jain, R. Langer, Engineering and physical sciences in oncology: challenges and opportunities. Nat. Rev. Cancer. 17, 659-675 (2017).
59. W. F. Sufferer, S. E. Hardin, R. W. Benson, L. Jerome Krovetz, G. L. Schiebler, Optical behavior of indocyanine green dye in blood and in aqueous solution. Am. Heart J. 72,345-350 (1966).
60. S. M. Mooi, B. Heyne, Size Does Matter: How To Control Organization of Organic Dyes in Aqueous Environment Using Specific Ion Effects. Langmuir. 28,16524-16530 (2012).
61. H. Wang, Q. Feng, J. Wang, H. Zhang, Salt Effect on the Aggregation Behavior of 1-Decyl-3-methylimidazolium Bromide in Aqueous Solutions. J. Phys. Chem. B. 114,1380-1387 (2010).
62. U. Toh, N. Iwakuma, M. Mishima, M. Okabe, S. Nakagawa, Y. Akagi, Navigation surgery for intraoperative sentinel lymph node detection using Indocyanine green (ICG) fluorescence real-time imaging in breast cancer. Breast Cancer Res. Treat. 153,337-344 (2015).
63. C. A. Schneider, W. S. Rasband, K. W. Eliceiri, NIH Image to ImageJ: 25 years of image analysis. Nat. Methods. 9,671-675 (2012).

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

What is claimed is:

1. A modified J-aggregate (MJ).

2. The MJ of claim 1, wherein said MJ comprises a J-aggregate forming dye.

3. The MJ of claim 1, wherein said MJ comprises a J-aggregate forming dye that has been functionalized.

4. The MJ of claim 1, wherein said MJ comprises a J-aggregate forming dye and a J-aggregate forming dye that has been functionalized.

5. The MJ of claim 3, wherein said J-aggregate forming dye has been functionalized with an azide group.

6. The MJ of claim 4, wherein said J-aggregate forming dye has been functionalized with an azide group.

7. The MJ of claim 2, wherein said J-aggregate forming dye is a cyanine dye.

8. The MJ of claim 2, wherein said J-aggregate forming dye is selected from the group consisting of indocyanine green (ICG), ICG-NHS, ICG-NH₂, pseudoisocyanine, merocyanine, bis(2,4,6-trihydroxyphenyl)squaraine, tetrtakis(4-sulfonatophenyl)-porphyrin, antimony(III)-phthalocyanine, copper phthalocyanine, perylene bismide, hypericin, subphtalocyanine, and combinations thereof.

9. An MJ produced by the process of mixing a J-aggregate forming dye and a J-aggregate forming dye that has been functionalized.

10. The MJ of claim 9, wherein the mixing a J-aggregate forming dye and a J-aggregate forming dye that has been functionalized occurs in the presence of a salt.

11. The MJ of claim 10, wherein said salt is selected from the group consisting of KCl, NaCl, MgCl₂, CaCl₂), NiCl₂, MnCl₂, EuCl₃, TbCl₃, and a salt from a rare earth element.

12. The MJ of claim 1, wherein the MJ is nanoscale, sub-micronscale, or microscale.

13. The MJ of claim 12, wherein the MJ is nanoscale.

14. An MJ conjugate of Formula I
MJ—E—D
Formula I
wherein
MJ is a modified J-aggregate;
E is a linker or a bond;
D is a functional moiety.

15. The MJ conjugate of claim 14, wherein said MJ comprises a J-aggregate forming dye.

16. The MJ conjugate of claim 14, wherein said MJ comprises a J-aggregate forming dye that has been functionalized.

17. The MJ conjugate of claim 14, wherein said MJ comprises a J-aggregate forming dye and a J-aggregate forming dye that has been functionalized.

18. The MJ conjugate of claim 16, wherein said J-aggregate forming dye has been functionalized with an azide group.

19. The MJ conjugate of claim 17, wherein said J-aggregate forming dye has been functionalized with an azide group.

20. The MJ conjugate of claim 14, wherein D has been attached to MJ by employing copper-free click chemistry with said azide group.

21. The MJ conjugate of claim 15, wherein said J-aggregate forming dye is a cyanine dye.

22. The MJ conjugate of claim 15, wherein said dye is selected from the group consisting of ICG, ICG-NHS, ICG-NH₂, pseudoisocyanine, merocyanine, bis(2,4,6-trihydroxyphenyl)squaraine, tetrtakis(4-sulfonatophenyl)-porphyrin, antimony(III)-phthalocyanine, copper phthalocyanine, perylene bismide, hypericin, subphtalocyanine, and combinations thereof.

23. The MJ conjugate of claim 14, wherein said MJ was produced by the process of mixing a J-aggregate forming dye and a J-aggregate forming dye that has been functionalized.

24. The MJ conjugate of claim 23, wherein the mixing a J-aggregate forming dye and a J-aggregate forming dye that has been functionalized occurs in the presence of a salt.

25. The MJ conjugate of claim 24, wherein said salt is selected from the group consisting of KCl, NaCl, MgCl₂, CaCl₂), NiCl₂, MnCl₂, EuCl₃, TbCl₃, NaOH, K₂PO₄, Mn(NO₃)₂, Ni(NO₃)₂ and a salt from a rare earth element.

26. The MJ conjugate of claim 14, wherein E is a linker comprising a copper-free click chemistry group.

27. The MJ conjugate of claim 14, wherein E is a linker selected from the group consisting of DBCO-PEG-NHS, DBCO-PEG-Biotin, DBCO-PEG-Thiol, DBCO-PEG-NH₂, DBCO-PEG-Mal, Thiol-PEG-NHS, Thiol-PEG-Biotin, Thiol-PEG-NH₂, Mal-PEG-NHS, Mal-PEG-NH₂, Mal-PEG-Acrylate, Thiol-PEG-Acrylate, DBCO-PEG-Folate, and combinations thereof.

28. The MJ conjugate of claim 14, wherein D comprises streptavidin, biotin, RGD, or a derivative thereof.

29. The MJ conjugate of claim 14, wherein D comprises a drug, a protein, a targeting protein, or an antibody.

30. A method of photoacoustic imaging at a target site comprising
providing the MJ of claim 1 at the target site; and
monitoring absorbance at the target site.

31. A method of photoacoustic imaging at a target site comprising
providing the MJ conjugate of claim 14 at the target site; and
monitoring absorbance at the target site.


SEQUENCE LISTING (1 sequence)

SEQ ID NO: 1 - protein, 5 aa, synthetic construct
  SITE 4: note = D-Phenylalanine
  SITE 5: note = Lysine amide bonded to biotin
RGDFK